(19) United States
(12) Patent Application Publication
(10) Pub. No.: US 2024/0158465 A1
(43) Pub. Date: May 16, 2024

(54) METHOD FOR INDUCING AND DETECTING SOLUBLE LOX-1 (sLOX-1) IN CULTURED BLOOD CLOTS

(71) Applicant: GEORGE MASON UNIVERSITY, Fairfax, VA (US)

(72) Inventors: Caroline Dieckmann Hoemann, Vienna, VA (US); Julia Alexa Leonard, Fairfax, VA (US); Virginia A. Espina, Rockville, MD (US); David Kepplinger, Fairfax, VA (US)

(73) Assignee: GEORGE MASON UNIVERSITY, Fairfax, VA (US)

(21) Appl. No.: 18/517,066

(22) Filed: Nov. 22, 2023

Related U.S. Application Data
(63) Continuation-in-part of application No. 18/321,738, filed on May 22, 2023.
(60) Provisional application No. 63/344,258, filed on May 20, 2022.

(51) Int. Cl. C07K 14/705; C12N 5/0787; G01N 33/50; G01N 33/68

(52) U.S. Cl. CPC C07K 14/7056; C12N 5/0642; G01N 33/5047; G01N 33/6893; C12N 2501/052; C12N 2501/90; C12N 2501/999; C12N 2523/00; G01N 2333/8125

(57) ABSTRACT

In an embodiment, present invention relates to a method of generating, ex vivo production of soluble Lox-1 (sLox-1), comprising: introducing a sample containing blood into a device; adding a coagulation enhancing material in the sample to form a cultured blood clot; incubating the cultured blood clot in the device at a temperature greater than 25° C. and less than 45° C. for at least 2 hours to allow production of Lox-1 from neutrophils of blood and to shed the sLox-1 outside the cultured blood clot; and collecting sLox-1 shedded in the device, wherein the method is configured to shed sLox-1 more than fresh blood.

### CROSS-REFERENCE TO RELATED APPLICATION

This application is a continuation in part of US non-provisional application 18/321,738, entitled, “METHOD FOR INDUCING AND DETECTING SOLUBLE LOX-1 (sLOX-1) IN CULTURED BLOOD CLOTS” which claims the benefit under 35 U.S.C § 119 of U.S. Provisional Application 63/344,258 filed on 20-MAY-2022, titled as “METHOD FOR INDUCING AND DETECTING SOLUBLE LOX-1 (sLOX-1) IN CULTURED BLOOD CLOTS”, which is hereby incorporated by reference in its entirety.

### FIELD OF THE INVENTION

This invention relates to the process of production of sLox-1, more particularly to production of sLox-1 in the cultured blood clot and the application of sLox-1.

### BACKGROUND OF INVENTION

Acute Respiratory Distress Syndrome (ARDS) can arise from trauma, sepsis, lung infection or transfusion [1]. Thrombocytopenia is a serious complication of ARDS that is partly brought on by inflammation-induced vascular permeability in the lung [2-5]. Mechanical ventilation is often needed to maintain blood oxygen levels but may contribute to platelet depletion [6]. Severe platelet depletion is a significant predictor of mortality [7]. A high burden of fibrin clot formation, as marked by elevated plasma D-dimer (>1 mg/mL), is also predictive of mortality [8, 9].

Patients with acute respiratory distress syndrome (ARDS) and neutrophilic inflammation often experience a drop in platelet levels and high mortality.

Biomarkers of neutrophil activation (lipocalin/LCN2) and neutrophil extracellular traps (NETosis) are harbingers of thrombocytopenia in ARDS [10-12]. Despite these important advances, there are currently no approved therapies that address immunothrombosis.

Like ARDS, coagulopathy has multifactorial initiators. Although less-studied, cardiomyopathy can develop after severe trauma [13]. A subset of Warfighters recovering from battlefield injuries may therefore face an additional long-term struggle with chronic heart disease. An understanding of why some individuals develop cardiomyopathy while sparing others is lacking.

In coagulopathy, platelets are activated by thrombin, by shear stress, by adhesion to sites of endothelial damage, ADP released from necrotic cells, and polyphosphates released from platelet alpha granules [14-18]. Excessive phosphatidylserine exposure on activated platelets or exosomes can activate the contact pathway and contribute to disseminated intravascular coagulation (DIC) [19, 20]. In the contact pathway, Factor XII unfolds on anionic surfaces to expose its active site [5, 11, 14, 21-24] which deploys soluble FXIa in the bloodstream along with bradykinin which drives edema [19, 25, 26]. Antithrombin is present at a 3-fold molar excess compared to prothrombin, and serves to “sop up” thrombin in an inactive thrombin-antithrombin (TAT) complex [27, 28].

It is currently unclear how to diagnose the risk or to treat trauma-induced cardiomyopathy. In patients with thrombocytopenia, is currently unclear how to diagnose low platelet counts caused by inadequate platelet production from low platelet counts caused by intravascular platelet activation (thrombosis).

### SUMMARY OF THE INVENTION

Stiffening of the arteries and the deposition of fatty plaques in the inner lining of the arteries contribute to hypertension which has a snowball effect of increasing vascular damage, ischemia, and cardiomyopathy. Quivering of the atrium that occurs in diastolic or systolic dysfunction can lead to pooling of the blood and clot formation on the side-wall of the atrium. These clots can break free and lodge in the lung or brain tissue causing transient ischemia, strokes, and sudden death.

Lox-1 has an established role in cardiovascular disease [29-36], and may have a role in trauma-induced cardiomyopathy [13].

In lung cancer patients, Lox-1 is identified as a biomarker of myeloid-derived suppressor cells (MDSCs) along with selected soluble mediators known to drive atrial hypertrophy and fibrosis (CXCL8, CXCL2, CCL2, IL1A, TNF, VEGFA) [37, 38]. A subset of these mediators were found in blood samples of patients with obstructive sleep apnea, a condition characterized by intermittent hypoxia/reoxygenation (IHR) [39-41].

These data suggest that Lox-1 and sLox-1 levels may be altered by hemorrhage, ischemia-reperfusion but in healthy human donors, endothelial cell expression of Lox-1 is scarce. The cells producing peripheral blood sLox-1 remain to be identified. Methods for producing sLox-1 are currently limited to complex systems involving cultured cells, mainly bacterial cells that cannot properly fold, dimerize or glycosylate sLox-1. Autologous sLox-1 with personalized glycosylations is not currently available for clinical use.

Further, research papers till the time of the present invention could not identify the cell source of sLox-1 and assumed that the endothelial cells were shedding sLox-1 into the bloodstream.

In an embodiment, present invention provides a method of generating, ex vivo production of soluble Lox-1 (sLox-1), comprising: introducing a sample containing a blood free from an anti-coagulant factor into a device; adding a coagulation enhancing material in the blood; incubating the device; forming a cultured blood clot in the device [42-44]; and shedding of the sLox-1 outside the cultured blood clot, wherein the method is configured to shed sLox-1 more than an anti-coagulated blood.

In an embodiment, the coagulating enhancing material comprises a lipopolysaccharide (LPS).

In an embodiment, the anti-coagulant factor comprises heparin, citrate, or ethylene diamine tetraacetic acid (EDTA).

In an embodiment, detecting one or more interleukins in the device.

In an embodiment, the one or more interleukins comprises IL-6, IL-8 and/or IL-18.

In an embodiment, addition of the inflammation enhancing material in the blood spikes shedding of sLox-1 into the device by about 20% to 60% more compared to a cultured blood clot free of the coagulation enhancing material.

In an embodiment, the method is configured to shed 0.5 ng to 50 ng of the sLox-1 per ml of the blood sample.

In an embodiment, the method is configured to produce an autologous sLox-1.

In an embodiment, the device is incubated at a temperature ranging from 20° C. to 40° C. for a time period ranging from 1 hour to 18 hours.

In an embodiment, the sLox-1 so sheds in the device is a personalized anti-coagulant or anti-thrombogenic agent.

In an embodiment, the device comprises a thrombus mimetic device.

In an embodiment, the method is configured to detect IL-8 and IL-6 concentrations in the cultured blood clot and the anti-coagulated blood.

In an embodiment, concentration of the sLox-1 in serum of the cultured clot compared to the anti-coagulated blood is from 0 pg/mL to about 50 ng/mL.

In an embodiment, concentration of the IL-8 in serum of the cultured clot compared to the anti-coagulated blood is from 50 pg/mL to 50 ng/ml.

In an embodiment, concentration of the IL-6 in serum of the cultured clot compared to the anti-coagulated blood is from 0 to 100 ng/mL.

In an embodiment, a device comprising a vacutainer tube and a means of heating to maintain temperature of the device at about 37° C., wherein the device is configured to screen agents that promote or inhibit one or more of the following: cell apoptosis, scramblase activity, flippase activity, ADAM17 activity, ADAM10 activity, alpha secretase activity, sLox-1 sheddase activity, neutrophil secretion, neutrophil-platelet aggregation, neutrophil degranulation, tumor necrosis factor activation.

In an embodiment, the device is configured to screen a drug, antibody, nanoparticle, microbial-derived component, biomaterial, neutraceutical/dietary supplement as causing an increase or decrease in cultured clot serum sLox-1 relative to untreated cultured clot serum sLox-1, without inducing cell necrosis.

In an embodiment, the drug is a chemotherapeutic agent intended to induce apoptosis.

In an embodiment, the drug is configured to decrease ADAM17 activity and TNF activation.

In an embodiment, the drug is configured to suppress TNF expression or activity.

In an embodiment, the drug is configured to suppress neutrophil degranulation.

In an embodiment, the drug is configured to suppress neutrophil secretion.

In an embodiment, the drug comprises a serine-threonine phosphatase inhibitor.

In an embodiment, the drug comprises beta glycerol phosphate.

In an embodiment, a portable testing kit that permits user to carry out method 1 at point-of-care or at home.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The accompanying drawings, which are included to provide further understanding of the present invention disclosed in the present disclosure and are incorporated in and constitute a part of this specification, illustrate aspects of the present invention and together with the description serve to explain the principles of the present invention. In the drawings:

FIG. 1 shows a model showing how to produce sLox-1 in cultured clots and proposed therapeutic function of sLox 1 released by thrombi to diminish Lox 1 activation by endothelial cells and clot formation in blood vessels (A) protein structure, (B) OLR1 (Lox-1 mRNA) levels in (1) fresh blood (2) fresh blood clot (3): cultured blood clot (4 hr at 37° C.), (C) sLox-1 protein release to cultured clot serum, and (D) model proposing how thrombus-derived sLox-1 could protect endothelium from activated platelets and thrombin, in a donor-dependent manner depending on the amplitude of clot-induced sLox-1 shedding.

FIG. 2 shows significant induction of (A) OLR1 (Lox-1 mRNA) in cultured blood clots and LPS/cultured clot and (B) sLox-1 shedding into cultured clot serum and LPS/cultured clot serum relative to fresh whole blood, fresh clot (45 min RT) and heparin blood. Numbers 1-6 in the figure on the X-axis refers to: 1) fresh blood plasma, 2) 45 minute RT clot, 3) cultured clot (4 h, 37° C.), 4) lipopolysaccharide/cultured clot (4 h, 37° C.), 5) bGP/cultured clot (4 h, 37° C.), and 6) cultured heparin blood (4 h, 37° C.).

FIG. 3. TEG assay of recalcified citrated plasma (260 μL) combined with 100 μL of (A) fresh clot serum (45 min RT) or (B) cultured clot serum (4 hours at 37 C) from the same donor. Cultured clot serum showed reduced thrombogenic activity relative to fresh serum. Y-axis: mm (clot tensile strength). X-axis: time (minutes). The white trace shows the amplitude of the clot tensile strength. The gray trace shows velocity of coagulation. This figure shows cultured clot serum has lower thrombogenic activity compared to fresh clot serum. Thromboelastography assay of pooled human citrated plasma (160 μL) combined with either (160 μL) fresh clot serum (A) or cultured clot serum (B) from the same donor, and then submitted to the TEG test with 20 μL of 200 mM CaCl₂. The clotting time was about 2-fold more, and the clot velocity and maximal amplitude reflecting clot tensile strength were around 2-fold less for cultured clot serum that contains 460 pg/mL of sLox-1 compared to fresh serum which had 0 pg/mL sLox-1.

FIG. 4 shows a cultured clot system according to an embodiment of the invention.

FIG. 5A shows high quality total RNA from fresh (anticoagulated) blood (FB) and cultured blood clot samples Polytron-homogenized into PAXgene buffer and lower quality total RNA from (condition 9) cultured clot samples vortex-mixed into PAXgene buffer.

FIG. 5B shows the yield of total RNA from fresh blood (FB) and cultured blood clot samples Polytron-homogenized into PAXgene buffer or (9) vortex-mixed into PAXgene buffer.

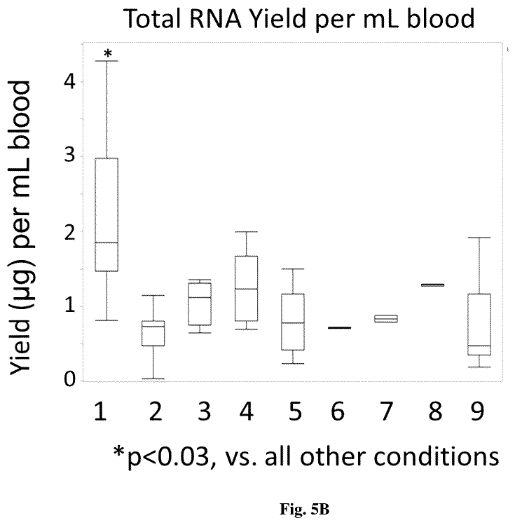

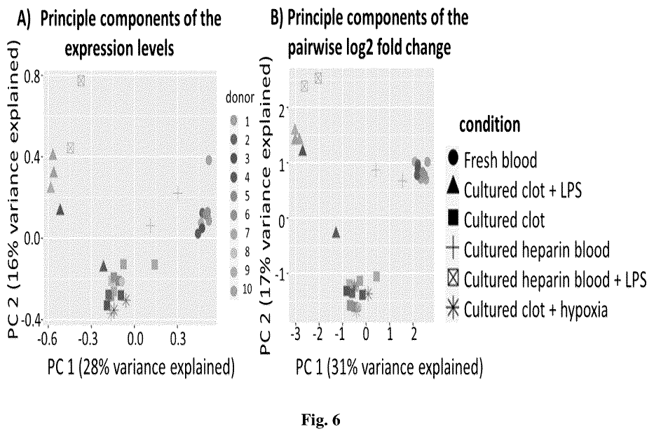

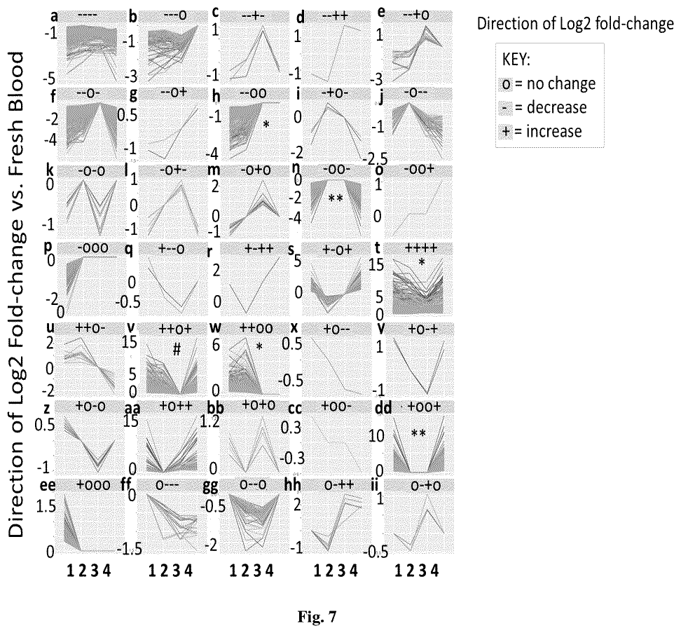

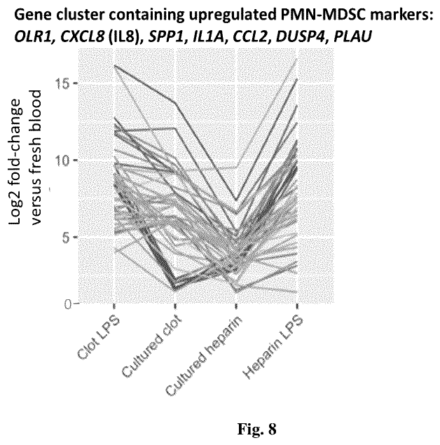

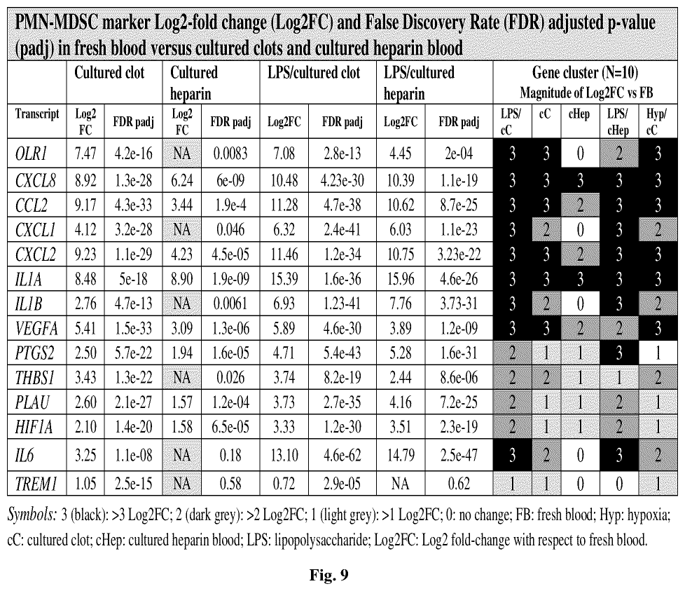

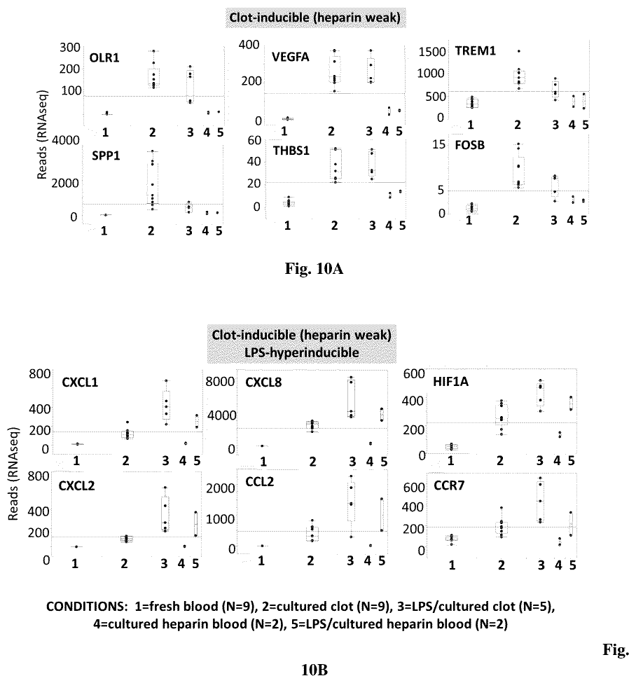

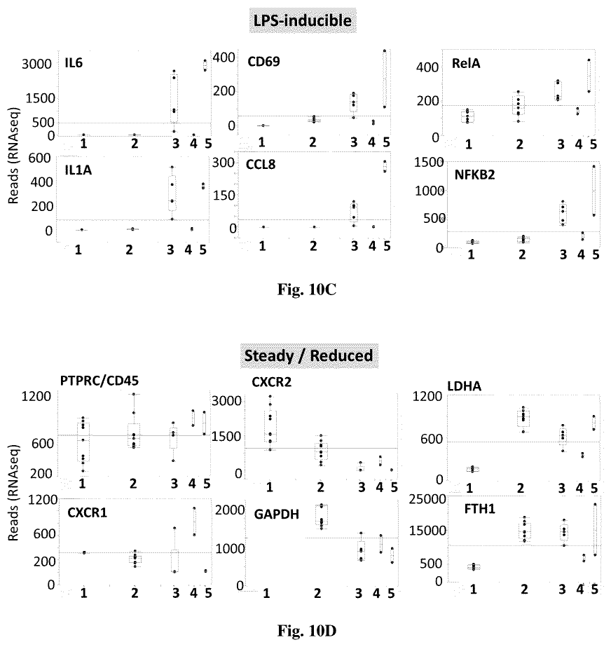

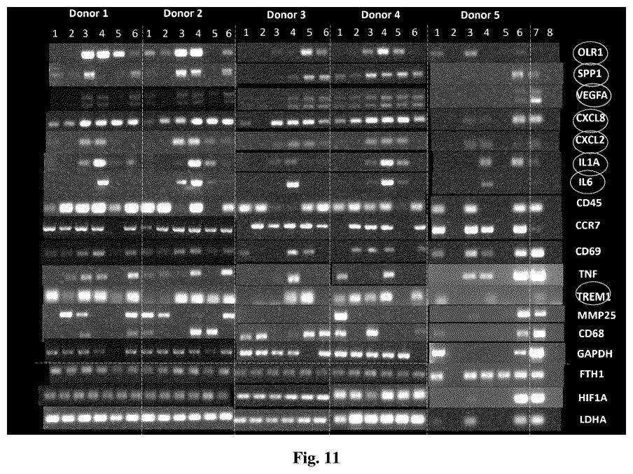

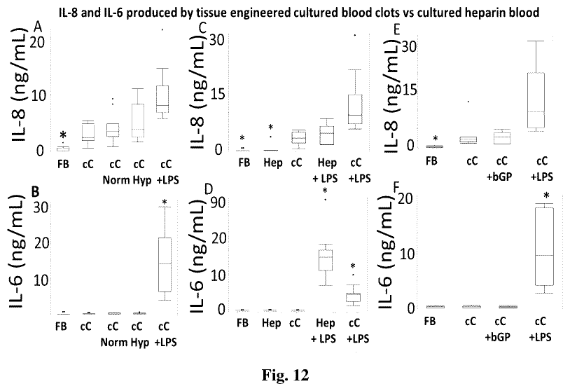

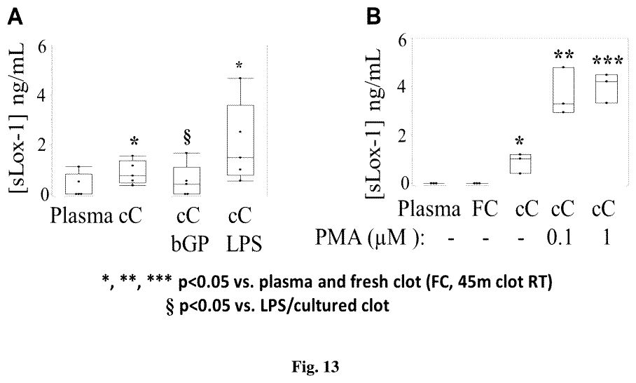

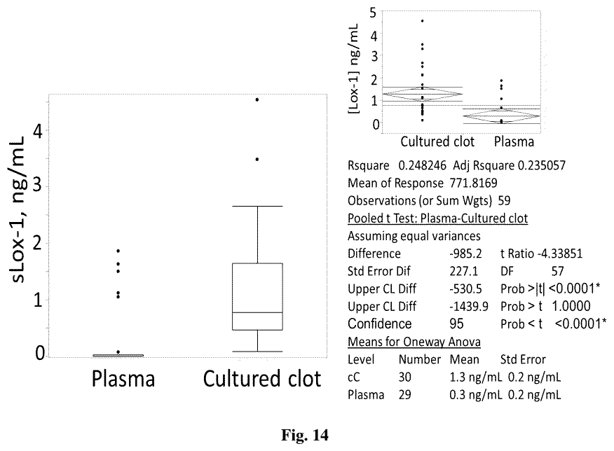

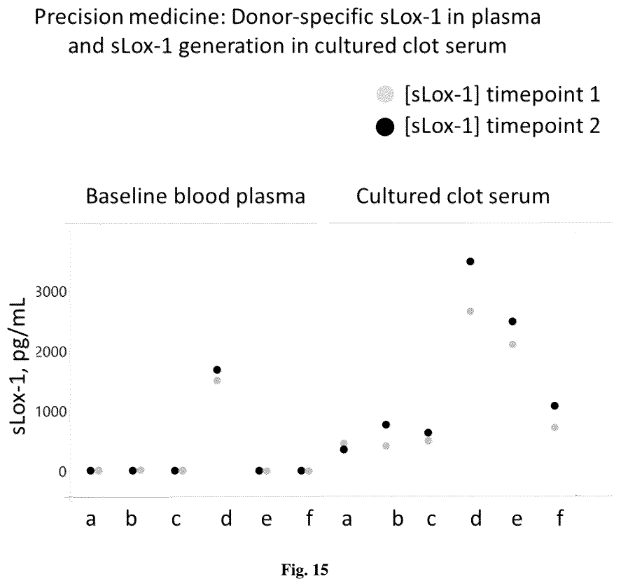

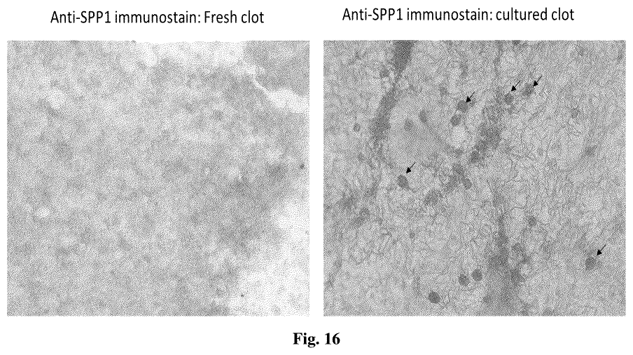

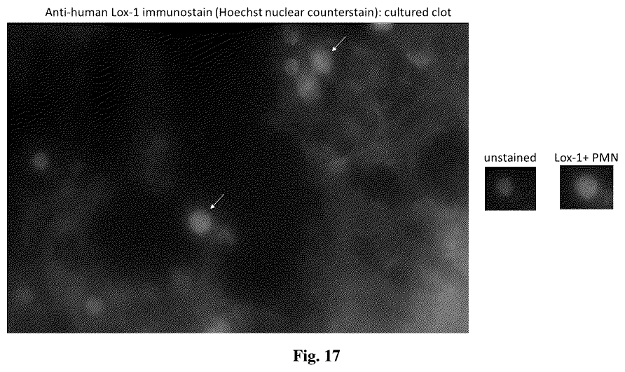

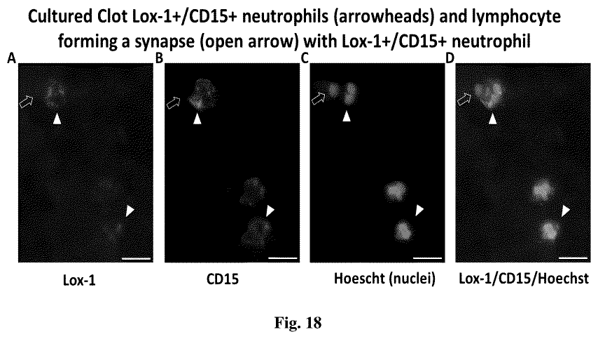

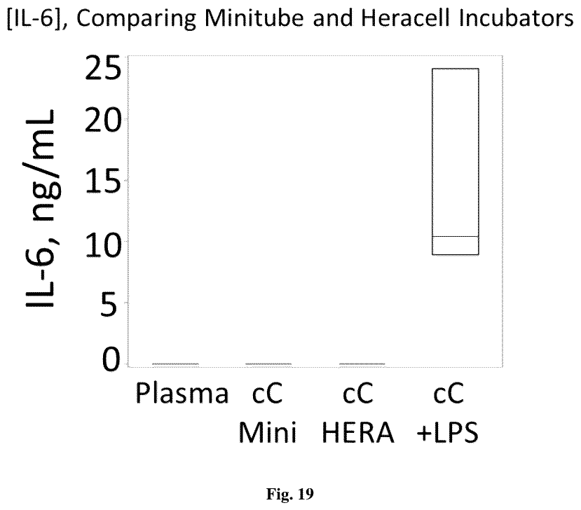

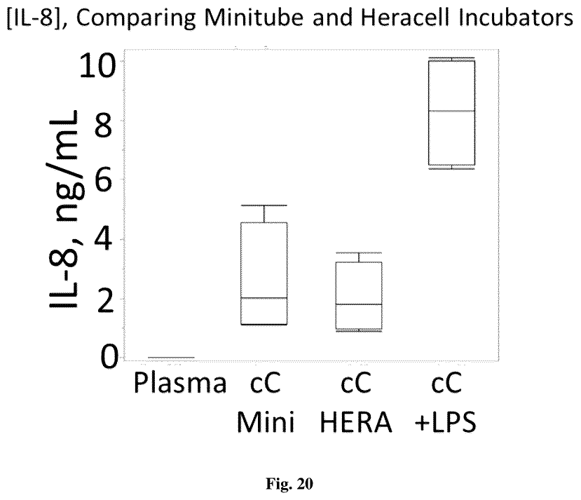

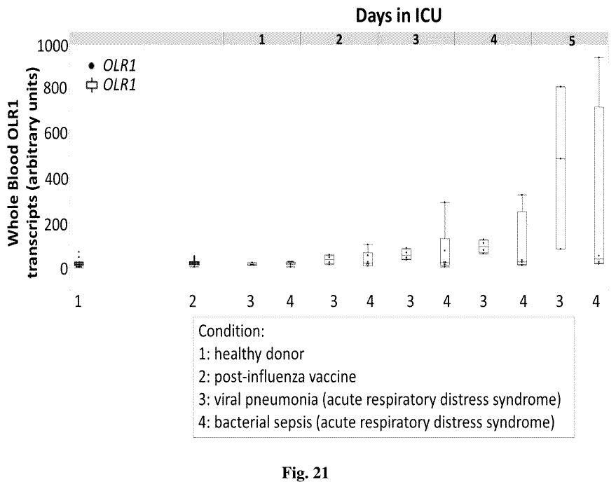

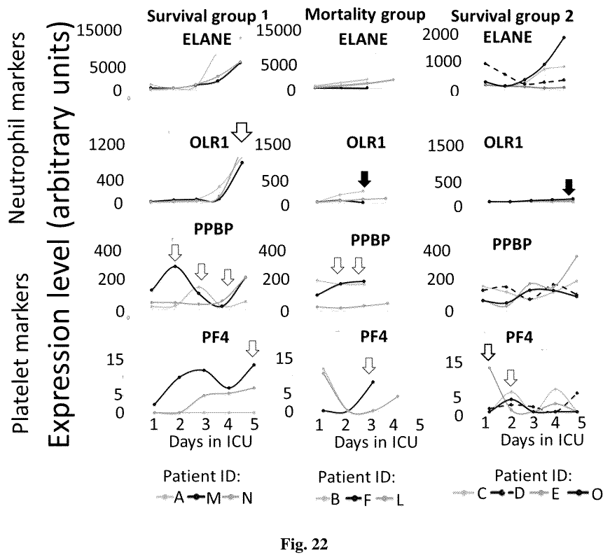

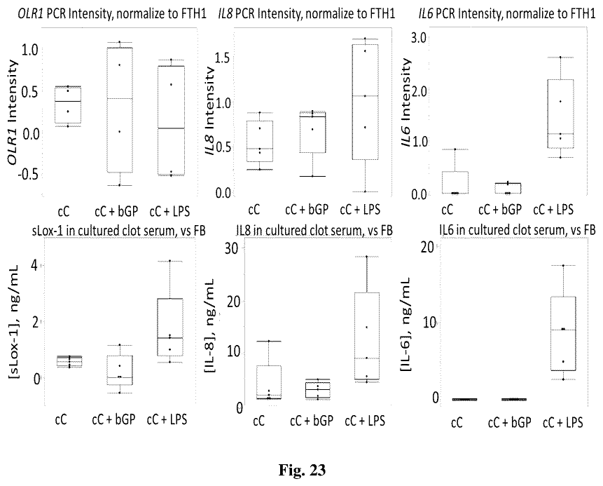

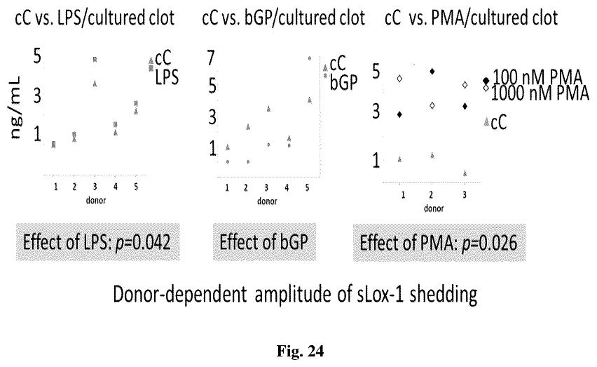

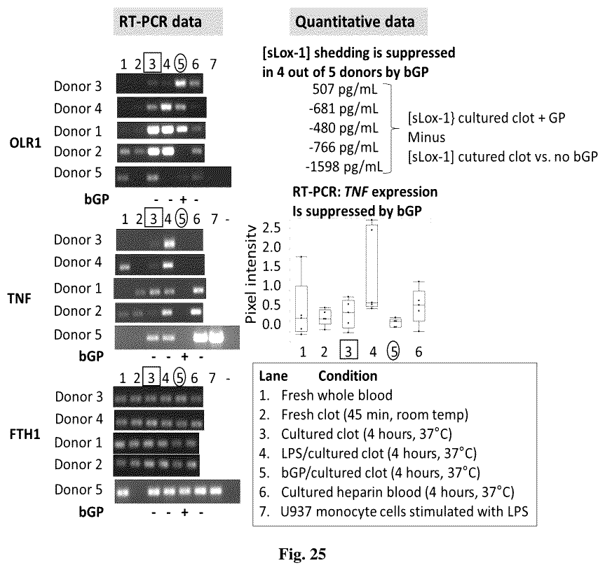

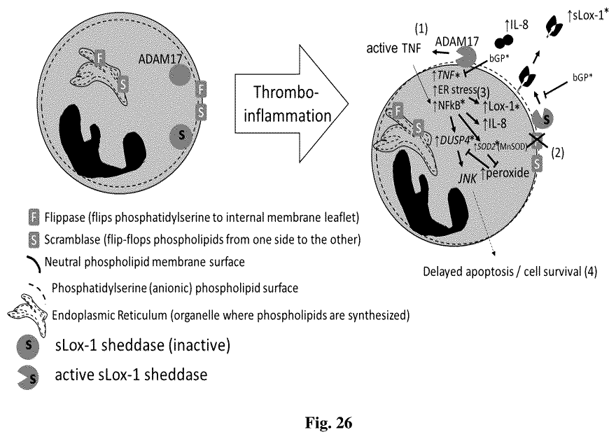

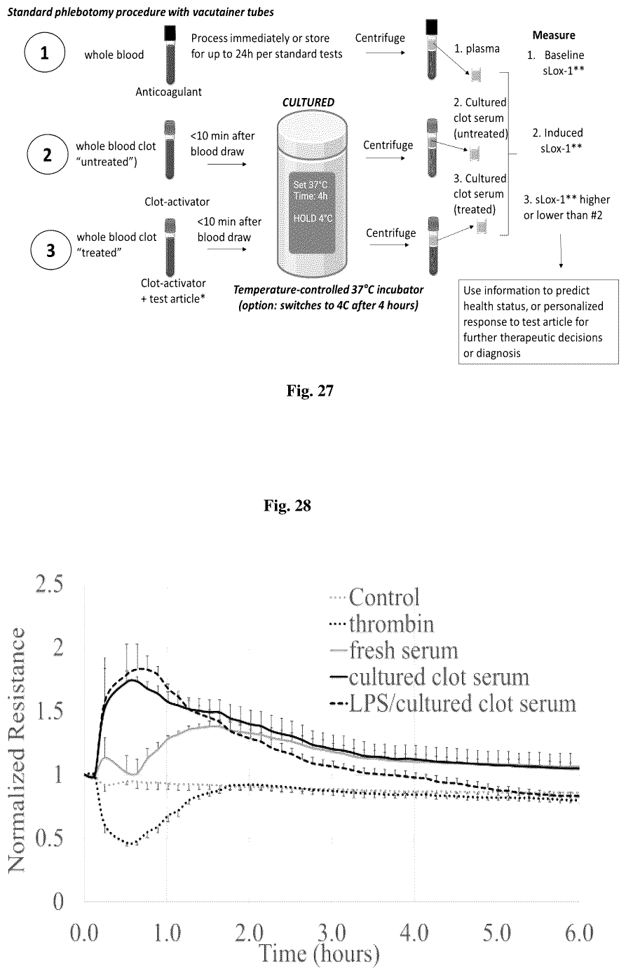

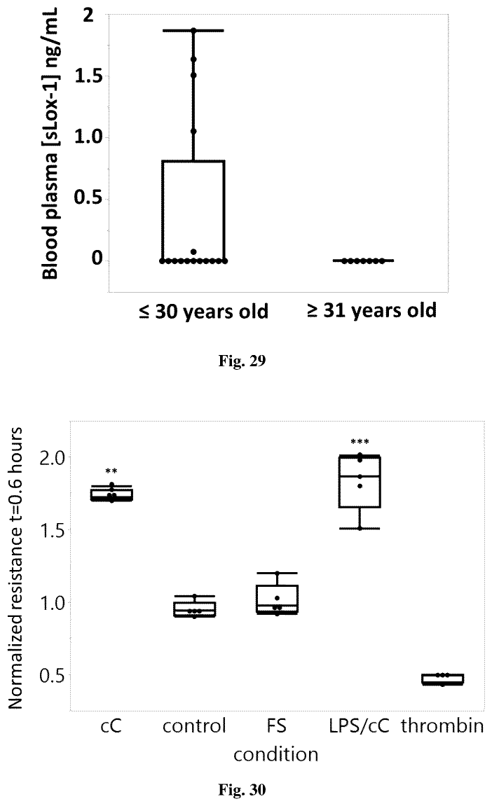

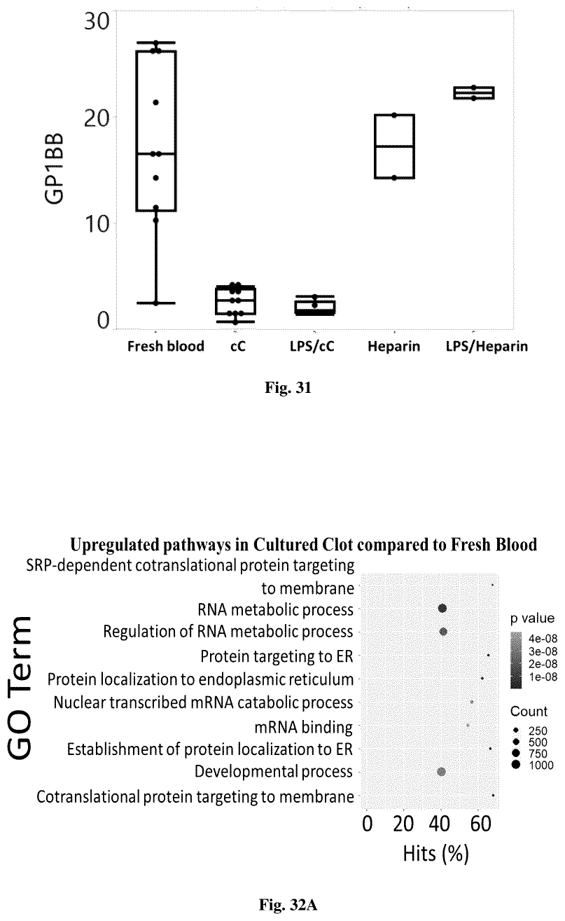

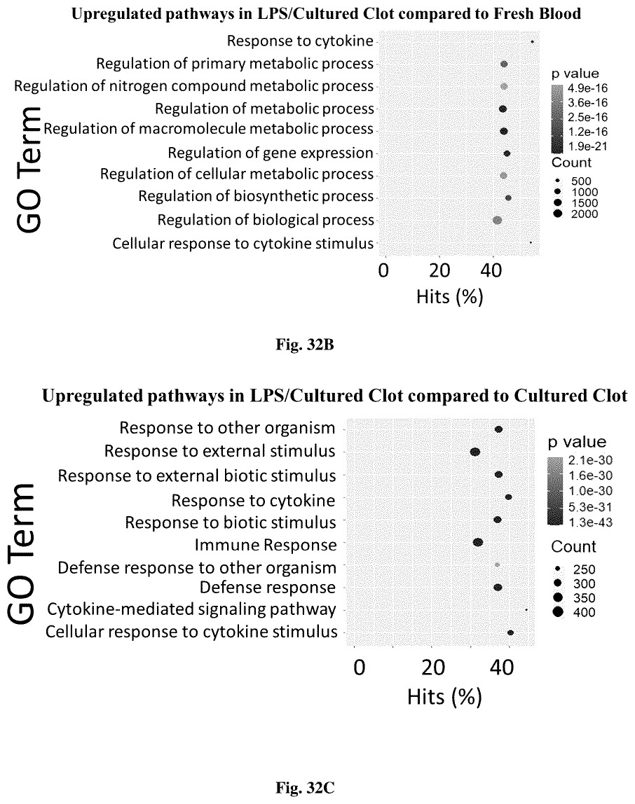

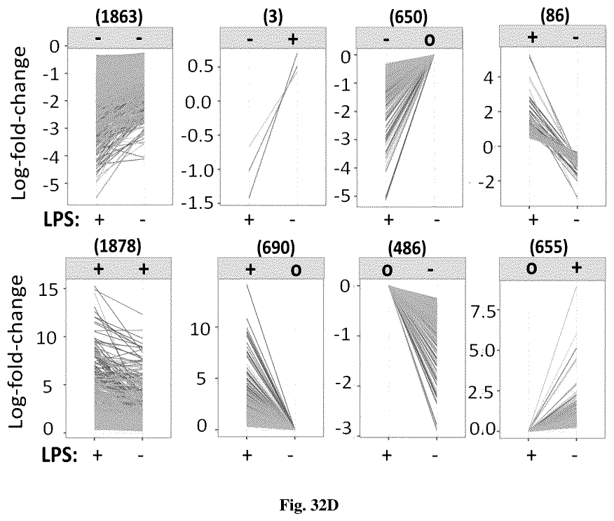

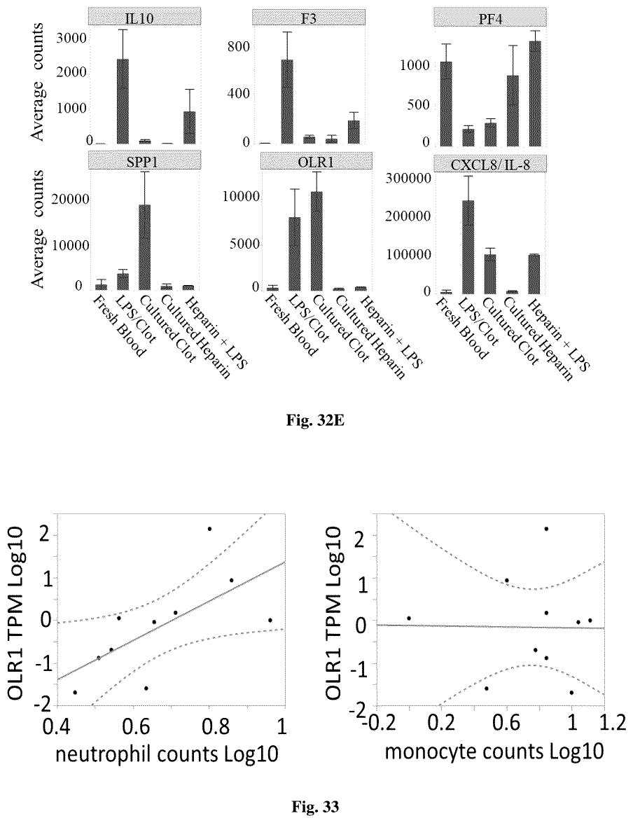

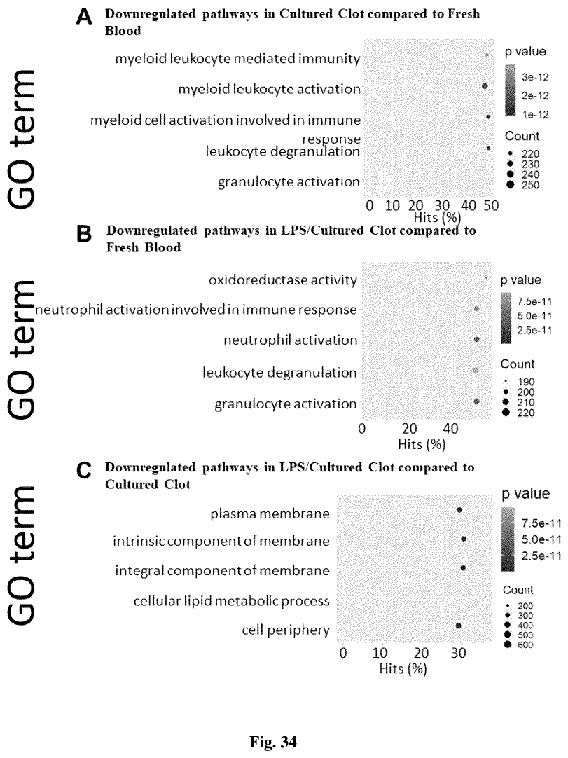

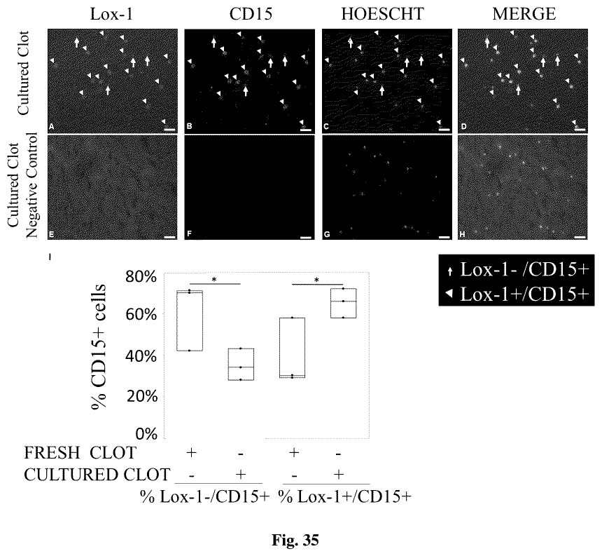

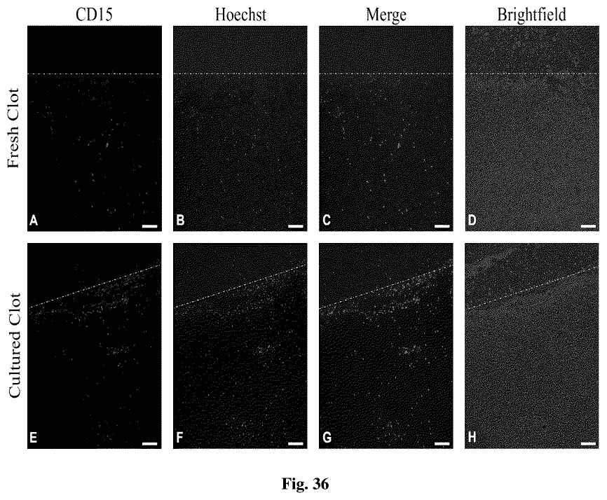

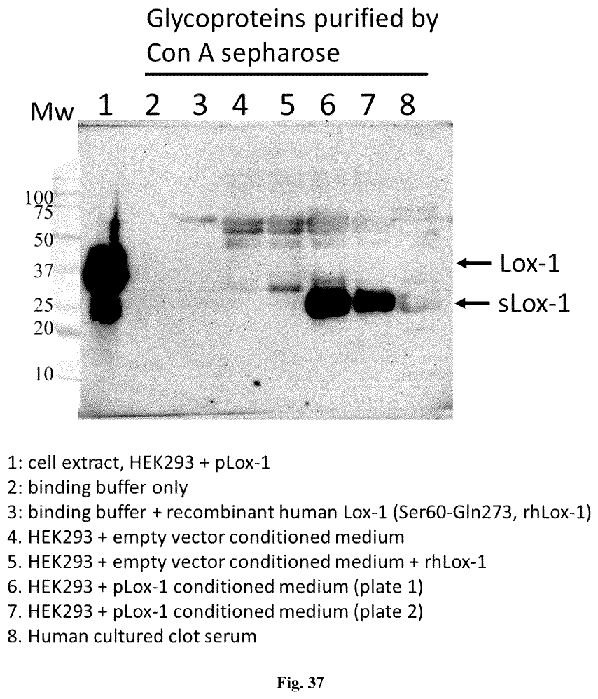

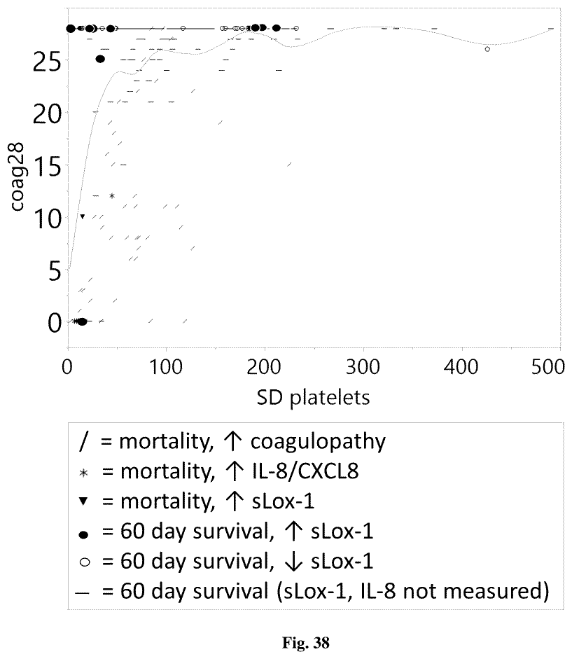

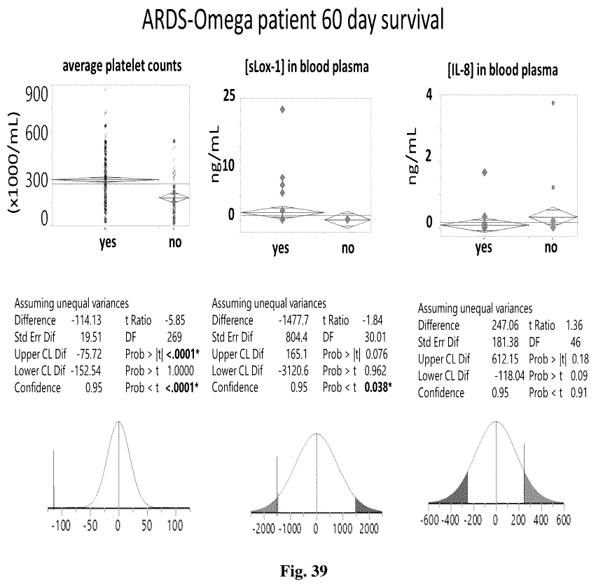

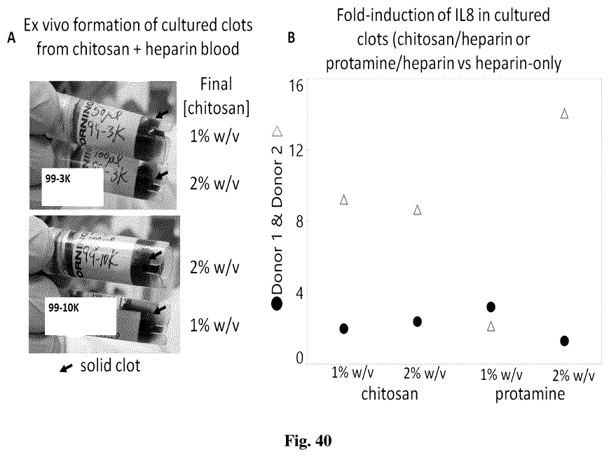

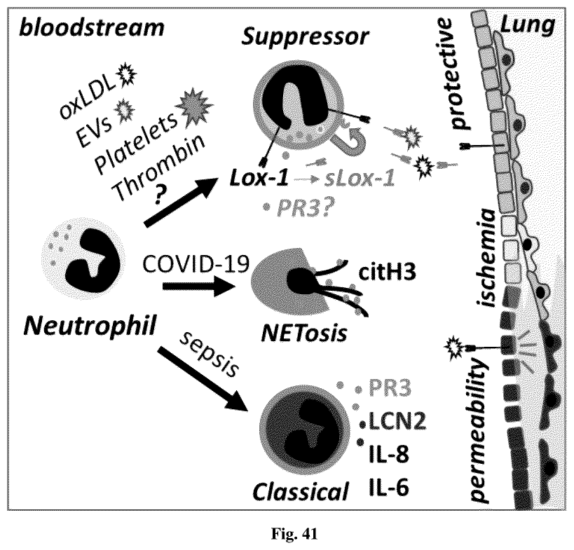

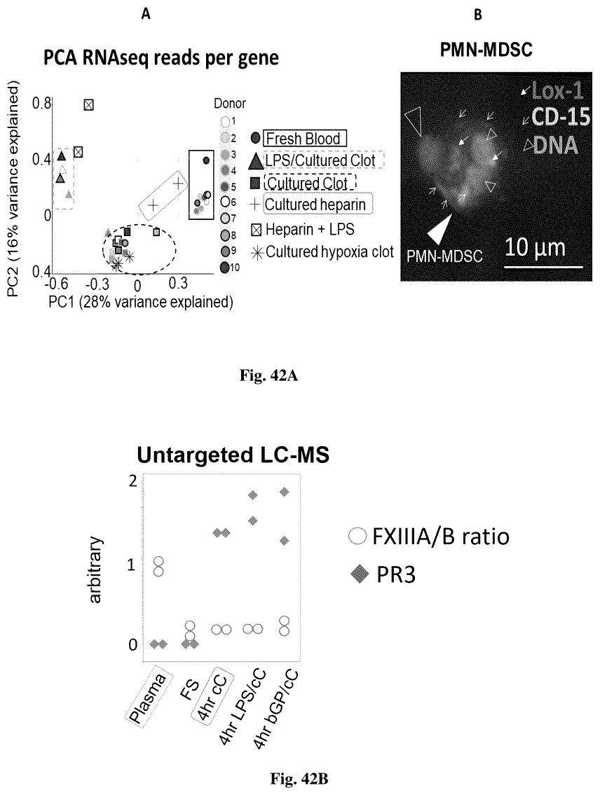

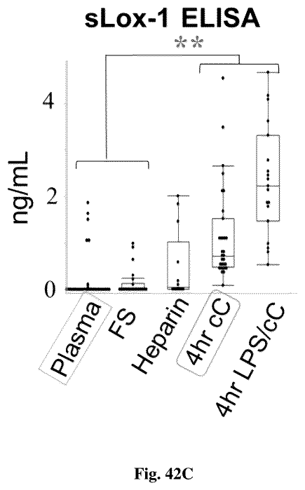

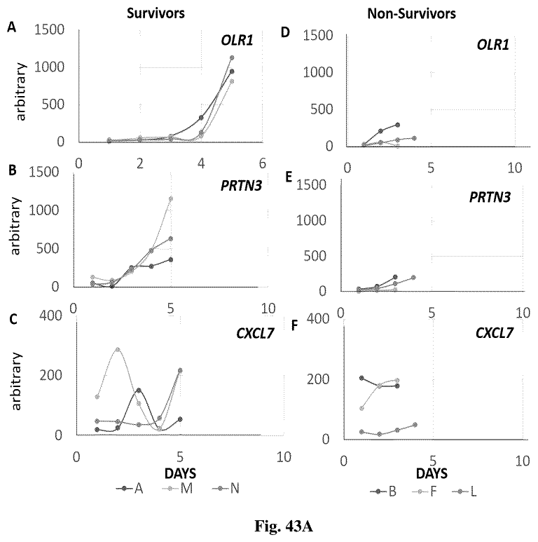

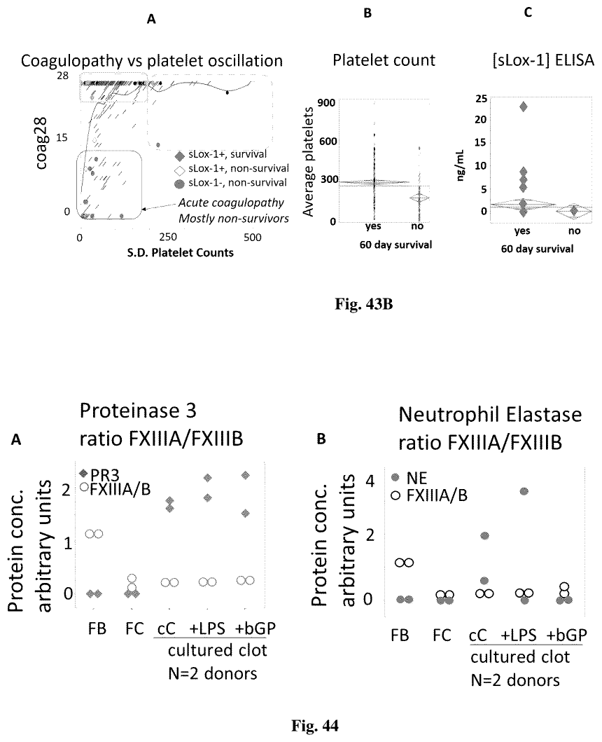

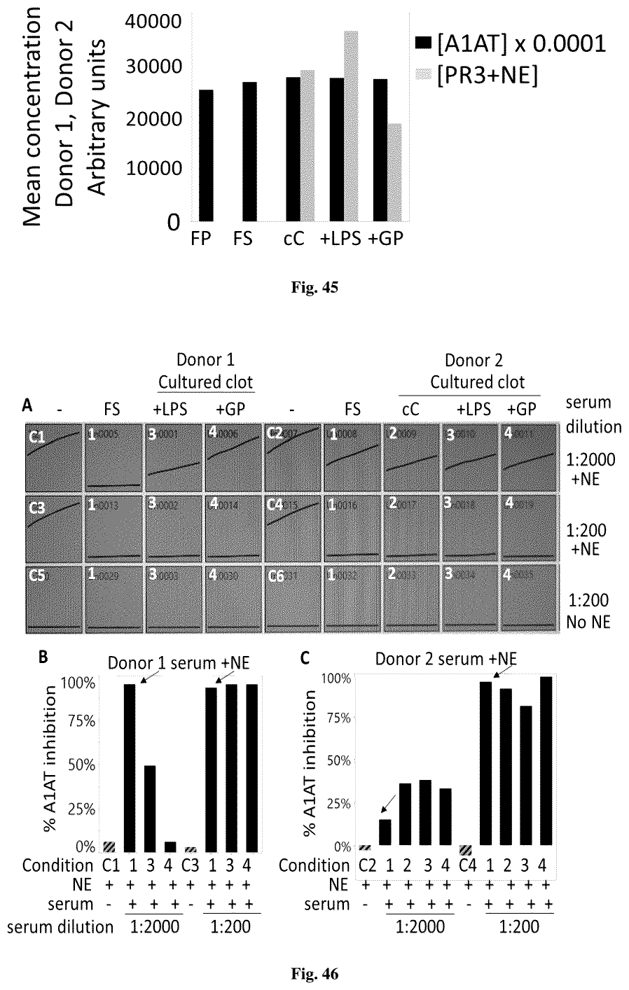

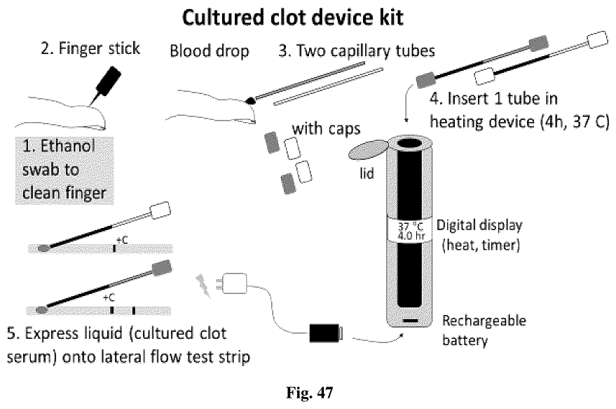

In FIG. 5A and 5B, ANOVA shows cultured clot vortex mixed into PAXgene reagent has significantly lower RNA integrity number (RIN) than all other conditions, p<0.006. FB has significantly higher RNA yield than all other conditions, p value p<0.03. FB N=21, RV, red vacutainer (RV) tube cultured blood clot N=17, Normoxia cultured blood clot N=9, Hypoxia cultured blood clot N=7, RV cultured clot treated with LPS N=15, Heparin (fresh anticoagulated blood) N=3, Cultured Heparin Blood N=3, Heparin-LPS: Cultured Heparin blood treated with LPS N=3, CC vortex (cultured clot vortex mixed into PAXgene) N=19.

In FIG. 5A and 5B, 1-9 on the X-axis refers to: 1) fresh blood, 2) cultured clot red vacutainer (RV) tube, 3) cultured clot, glass tube, Normoxia, 4) cultured clot, glass tube, Hypoxia, 5) cultured clot+LPS, 6) heparin blood, 7) cultured heparin blood, 8) cultured heparin blood+LPS, and 9) represents cultured clot, vortex mix into PAXgene reagent.

FIG. 6 provides scores on the first two principal components (PCs) of the (A) expression levels and (B) the scores on the first two PCs of the pairwise log2 fold changes. Healthy non-fasting and consenting volunteers provided blood samples to analyze fresh blood (N=10), cultured clots (4 h at 37° C., N=10), cultured clots with added 100 ng/mL lipopolysaccharide (4 h at 37° C., LPS, N=5), cultured heparin blood with and without 100 ng/mL LPS (4 h at 37° C., N=2). PCA analyses showed that cultured clot global gene expression varied from that of fresh blood and LPS-stimulated samples. In one donor out of 10, the fresh blood profile clustered with cultured clot samples. LPS stimulated a distinct response in 4 out of 5 donors from cultured clots according to PCA components of the expression levels.

FIG. 7 provides cluster analysis of differentially expressed genes vs fresh blood according to the direction of Log2 fold-change in gene expression, in (left to right): LPS/cultured clots (n=5), cultured clots (n=10), cultured heparin (n=2), LPS/cultured heparin (n=2). Symbols illustrate gene clusters identified as upregulated or downregulated in cultured clot (*) refers to cultured clot-specific response genes (panels h, t, w)), (#) refers to cultured clot and LPS -induced genes (panel v), and (**) refers to LPS-specific response genes (panels n, dd).

Numbers (1-4) on x-axis refers to: 1) cultured clot+LPS, 2) cultured clot, 3) cultured heparin blood, and 4) cultured heparin blood+LPS. Number of genes in each cluster: a) 378, b) 95, c) 3, d) 1, e) 18, f) 912, g) 2, h) 587, i) 3, j) 50, k) 10, l) 5, m) 11, n) 356, o) 1, p) 190, q) 2, r) 1, s) 69, t) 594, u) 11, v) 757, w) 549, x) 1, y) 2, z) 9, aa) 56, bb) 5, cc) 1, dd) 486, ee) 128, ff) 16, gg) 80, hh) 6, ii) 3.

FIG. 8 provides log 2 fold-change (LFC) for (1) cultured clot+LPS, (2) cultured clot, (3) cultured heparin blood and (4) cultured heparin blood+LPS (as indicated at the bottom of the figure). The cluster included OLR1 (mRNA encoding Lox 1) and genes previously identified by Veglia et al (2021) Nature Reviews Immunology 21:485-498, as being activated in human myeloid derived suppressor cells (CXCL8/IL8, SPP1, IL1A, CCL2, DUSP4, PLAU). Following genes are included in this cluster: AK4, F3, RGS1, IL10, LAMB3, G0S2, BANAM2-AS1, IL1RL2, ILIA, IL1B, NR4A2, CCL20, HRH1, PPRAG, CXCL8, CXCL3, CXCL2, EREG, AREG, SPP1, HBEGF, C6orf223, HILPDA, CLEC5A, EGR3, DUSP4, NR4A3, TNFSF15, PLAU, OLR1, GPR84, GJB2, C15orf48, CCL22, CCl2, CCl7, CCl3, KCNH4, COL1A1, SNAI1, SLCO4A1, LIF, OSM, CCL3_1, CCL3L3_1, CCL3L3_2, CCL4L2_2, CCL3L1, CCL4L1.

FIG. 9 shows PMN-MDSC marker Log2-fold change (Log2FC) and False Discovery Rate (FDR) adjusted p-value (padj) in fresh blood versus cultured clots and cultured heparin blood.

FIGS. 10A, 10B, 10C, 10D show fresh blood is devoid of markers of PMN MDSCs in 9 out of 10 healthy donors whereas all cultured blood clots develop expression of markers previously associated with PMN MDSCs including OLR1 (Lox 1), SSP1, CXCL8, CCL2, IL6, CD69, CXCL1, VEGFA, TREM1, CXCL2, THBS1, and IL1A (figure shows N=9 healthy human donors). Differences between fresh blood (1), cultured clot (2), LPS/cultured clot (3), cultured heparin blood (4), LPS/cultured heparin blood (5) samples were analyzed according to TPM read counts by RNAseq (line: grand mean; mean diamonds: mean and quartiles; points). Pooled t Test showed significant differences of the mean comparing cultured clot with fresh blood with p≤0.001 for all markers except for IL6 (p<0.05) and IL1A (p<0.01) versus cultured clot no LPS. Gene expression profiles were categorized as being induced by clot culture, by clot culture and more induced by LPS, only induced by LPS, or highly expressed/reduced. OLR1 expression levels correlated with FosB levels (i.e. AP-1, R∧2=0.75, p<0.0001) but not with RelA expression levels. [Conditions: 1=fresh blood (N=9), 2=cultured clot (N=9), 3=LPS/cultured clot (N=5), 4=cultured heparin blood (N=2), 5=LPS/cultured heparin blood (N=2).

FIG. 11 shows RT-PCR validation of RNA sequencing experiment shows that OLR1 and MDSC markers are induced in cultured clots relative to fresh blood and fresh blood clots in blood samples from N=5 non-fasting healthy consenting male and female donors with blood types A or O; Lewis Leb, Lea or null; Fya/Fyb, Duffy Fya, Fyb, or Fya/Fyb, secretor and nonsecretor. Lanes: 1. Fresh whole blood (PAXgene); 2. Fresh blood clot (45 min, RT); 3. Cultured clot (4 h, 37° C.); 4. LPS/cultured clot (4 h, 37° C.); 5. Beta-glycerol phosphate cultured clot (4 h, 37° C.); 6. Cultured heparin blood (4 h, 37° C.). OLR1 was induced in all cultured clots (4) and in all cultured clots+LPS (lane 5). IL6 was only induced by LPS (lane 4). TNF was specifically suppressed in cultured clots treated with 10 mM disodium beta glycerolphosphate (lane 5), without suppressing expression of CXCL8/IL-8, or housekeeping genes FTH1 and LDHA.

FIG. 12 shows compared to blood plasma and fresh clot serum, cultured clot serum shows elevated CXCL8/IL8 but not IL-6 protein. Cultured LPS/clot serum shows elevated CXCL8/IL-8 and IL-6. Adding beta glycerol phosphate to the clot does not alter clot induced CXCL8/IL-8. [Key: FB: fresh blood plasma, cC: cultured clot (4 h, 37° C.), Norm: cultured clot under Normoxia, Hyp: cultured clot under Hypoxia, Hep: heparin blood culture (4 h, 37° C.), +LPS: +100 ng/mL lipopolysaccharide, +bGP: +10 mM disodium beta glycerol phosphate].

FIG. 13 shows sLox-1 serum levels, for the samples shown in FIG. 12, panels E and F, and PMA-treated cultured clots. This is the enabling data for beta glycerol phosphate and PMA treated blood clots. Beta-glycerol phosphate (bGP) suppressed sLox-1 shedding and PMA enhanced sLox-1 shedding into cultured clot serum. § sLox-1 in serum from cultured clot (cC)+bGP was only significantly lower than sLox-1 in serum from cC+LPS.

FIG. 14 shows OLR1 gene expression in cultured blood clots leads to sLox-1 production in cultured clot serum (ELISA assay, N=23 biological replicates and N=7 technical replicates). ELISA shows that sLox-1 is induced and released to the serum of cultured clots, analysis of n=28 donors with n=23 biological replicates and n=7 technical replicates. Technical replicates were reproducible over time and distinct blood draws. sLox-1 was elevated in baseline plasma 4 out of 23 healthy donors (blood type A or type O), and in these donors, sLox-1 was more significantly induced in cultured clot serum (t ratio=−4.38 for plasma—cultured clot sLox-1 assuming equal variances, p<0.0001; 95CI: −53.4 to 595 pg/mL plasma, 937 to 1574.9 pg/mL cultured clot).

FIG. 15 shows precision medicine: Donor specific sLox 1 in plasma and sLox 1 generation in cultured clot serum. [sLox1] pg/mL for 6 human donors (a to f) for 2 distinct blood samples collected weeks to years apart.

FIG. 16 shows SPP1 expressing mononuclear cells are detected in cultured clots (arrows, dark brown signal, HRP substrate). SPP1 is considered a biomarker of PMN-MDSCs.

FIG. 17 shows anti-human Lox-1 immunostain of cultured clot and representative images of unstained and Lox-1⁺ stained cells. Lox-1 expressing mononuclear cells and polymorphonuclear cells are detected in cultured clots (white arrow, grey signal, ABC AP red substrate, epifluorescence microscopy, DAPI counterstained nuclei).

FIG. 18 shows cultured clot Lox-1⁺/CD15⁺ neutrophils (arrowhead) and lymphocycte forming a synapse (open arrow) with Lox-1+/CD15⁺ neutrophil.

FIG. 19 shows cultured clots can be produced using any incubator or condition that maintains the clot around 37° C. In this example, vacutainer tubes were placed in a humidified cell culture incubator (Heracell) or in a Minitube® portable 37° C. incubator. [cC=Cultured clot].

FIG. 20 show cultured clots can be produced using any incubator or condition that maintains the clot around 37° C. In this example, vacutainer tubes were placed in a humidified cell culture incubator (Heracell) or in a Minitube® portable 37° C. incubator. [cC=Cultured clot].

FIG. 21 shows averaged whole blood OLR1 transcript levels in whole blood RNA from patients with sepsis or viral pneumonia induced Acute Respiratory Distress Syndrome (ARDS). The averaged data reveal an effect of day, but do not reveal an effect of disease condition or disease severity. (meta-analysis of GEO data from Parnell et al, Shock, 2013; 40:166-174) [45, 46]. [conditions: 1: Healthy donor, 2: post-influenza vaccine, 3: viral pneumonia (acute respiratory distress syndrome), 4: bacterial sepsis (acute respiratory distress syndrome)].

FIG. 22 shows analysis of personalized regulation of OLR1 in whole blood transcript levels in patients with Acute Respiratory Distress Syndrome, during 6 days of ICU oxygen ventilation suggests a potential thromboprotective effect of OLR1 expression. Bacterial sepsis (A, B, C, D, E), viral pneumonia (L, M, N, O): OLR1 levels rose in patients A, M, N following or concomitant with platelet markers. ELANE: neutrophil elastase; OLR1: oxidized LDL receptor 1 (PMN-MDSC marker); PPBP: pro-platelet basic protein; PF4: platelet factor 4; Patient condition after day 6 was not reported. Metadata analysis of RNAseq data from samples published by Parnell et al. Identifying Key Regulatory Genes in the Whole Blood of Septic Patients to Monitor Underlying Immune Dysfunctions. Shock; 2013; 40:166-174 [45, 46].

FIG. 23 shows RT-PCR of transcript levels (OLR1, IL8, IL6) in cultured clots from healthy donors, and matching soluble factor released to cultured clot serum, corrected to fresh blood. Averaged data show that OLR1 is induced in the cultured clot and that sLox-1 shedding to the serum is increased by LPS and inhibited by a phosphatase inhibitor. Parallel analysis of IL-8/CXCL8 in the cultured clot serum can be used to document test article cytocompatibility. The absence of IL6 in donor fresh blood can be used to verify there is no sepsis or bacteremia, and lack of IL-6 induction in the cultured clot serum can be used to identify sterile inflammation and test article purity (lack of endotoxin response).

FIG. 24 shows personalized regulation of sLox-1 sheddase activity in cultured blood clots from healthy donors by endotoxin or by a drug (phosphatase inhibitor, beta glycerol phosphate, bGP, or phorbolmyristate acetate, PMA). These data show that the cultured clot device could be used as a precision medicine device to test for drug-induced responses that aim to reduce or increase the abundance of cells expressing Lox-1 (i.e., PMN-MDSCs), or alter sLox-1, or mediators upstream of sLox-1 shedding, including proteinase 3 or elastase activity, ADAM17 sheddase activity, phosphatidylserine exposure which is needed for ADAM17 activation, apoptosis which causes phosphatidylserine exposure, or TNF activity which depends on ADAM17. ADAM17 is also called TNF activating convertase enzyme, TACE.

FIG. 25 shows RT-PCR data for OLR1, FTH1, TNF from different types of clots. “−” identifies the negative control lane (no cDNA template), and quantitative data of sLox-1 in cultured clot+bGP serum vs. cultured clot no bGP.

FIG. 26 shows model of sLox-1 shedding as a biomarker of upstream/parallel cellular activities. 1) Sommer et al. Nature Comm 2016; 7: 11523. 2) Sakuragi et al. PNAS 2019. 116 (8) 2907-2912; 3) Condamine et al. Science Immunology 2016 Aug;1(2); 4) Kamata et al. 2005. Cell 120: 649-661 [37, 47-49].

FIG. 27 shows standard phlebotomy procedure with vacutainer tubes.

FIG. 28 shows cultured clot serum (with or without added LPS) had enhanced endothelial barrier-enhancing effects compared to fresh clot serum. Blood plasma, fresh serum, cultured clot serum and LPS/cultured clot serum were all collected from the same donors for the TER assay. The data were compiled as the average and standard deviation of N=5 tests (and N=4 tests for thrombin).

FIG. 29 shows sLox-1 levels according to age. Out of 23 healthy donors, elevated sLox-1 in baseline blood plasma was observed only in donors under 30 years old (N=16 below 30 years old vs N=7 over 30 years old; Non-significant difference, p=0.16). These data are enabling data for FIG. 27 where clot-induced sLox-1 production requires comparison with fresh blood plasma or fresh clot serum.

FIG. 30 shows TER assay of 5 tests of samples collected from 4 donors showing significantly greater barrier-enhancing effect of cultured clot serum (cC) and LPS/cultured clot serum (LPS/cC) compared to control and fresh serum (FS) at 0.6 hours post-stimulation (**, *** p<0.0001 vs control, FS, thrombin, Tukey Honest Significant Differences All Pairwise Comparisons). Thrombin induced a transient loss of barrier function.

FIG. 31 shows platelet-specific receptor GP1BB mRNA is downregulated in cultured clots vs fresh blood (based on read counts from bulk RNAseq, N=10 healthy nonfasting donors). Loss of platelet-specific mRNA read counts in cultured clots is evidence to suggest that platelet mRNA becomes labile after platelet activation in the clot.

FIG. 32A shows upregulated pathways in cultured clot compared to fresh blood.

FIG. 32B shows upregulated pathways in LPS/cultured clot compared to fresh blood.

FIG. 32C shows upregulated pathways in LPS/cultured clot compared to cultured Clot.

FIG. 32D shows cluster analysis of differentially expressed genes vs fresh blood according to the direction of Log2 fold-change in gene expression for cC or LPS/cC. Numbers over each panel refer to number of genes in each cluster and symbols in grey shaded area (−, +, o) refer to direction of change in gene expression relative to fresh blood. Cultured clots and LPS/cultured clots (LPS/cC) showed distinct shifts in metabolic processes, organelle function and cytokine response when comparing cC or LPS/cC to fresh whole blood and LPS/cC to cC.

FIG. 32E shows specific increase in OLR1 and specific decrease in platelet mRNA (PF4) in cultured clots.

FIG. 33 shows Linear regression (log-log) of fresh blood absolute leukocyte counts as predictors of fresh blood OLR1 expression (TPM, RNAseq) (N=10). OLR1 expression was positively associated with neutrophil counts (R-squared=0.43, p=0.04) but not monocyte counts (R-squared=1.0, p=0.96).

FIG. 34 Cultured clots and LPS/cultured clots showed downregulation of pathways involved in neutrophil activation and degranulation after 4 hours of culture at 37° C. GO plots of downregulated pathways in A) Cultured clot versus FB, B) LPS/cultured clot versus FB, C) LPS/cultured clot versus cultured clot. Count refers to the number of genes assigned to a given pathway that were altered.

FIG. 35 shows all Lox-1⁺ clot cells were CD15⁺ and the percent of CD15⁺ cells co-expressing Lox-1 increased during 4 hours of clot culture. Panels A) to D) show example double immunostain for (A) Lox-1, (B) CD15, (C) Hoechst nuclear stain, and (D) merge where all Lox-1⁺ cells (white arrows) are also CD15⁺ however some CD15⁺ cells (open arrowhead) were Lox-1⁻. Panels E) to H) show negative control immunostain where nonspecific (E) goat IgG or (F) mouse IgM was used, (G) Hoechst stain and (H) merge. Scale bars are 20 μm. (I) Percentage of CD15⁺ cells that also expressed Lox-1 in fresh clots and cultured clots from 3 healthy donors increased with clot culture (* p<0.05, Student's t test for unequal variances, median, min-max, N=3). Methods: Transverse fresh clot and cultured clot tissue sections from 3 healthy donors were imaged at 20× magnification at the top, middle and bottom of the clot and scored for Lox-1 (arrow) and CD15 (arrowhead) expression. The similar number of total cells in the fresh clot (38±3 cells per field) and cultured clot (47±8 cells per field) supports the notion that CD15⁺ cells acquired Lox-1 expression rather than selective apoptosis of Lox-1⁻/CD15⁺ cells during clot culture.

FIG. 36 shows CD15⁺ neutrophils migrated toward the surface of the cultured clot. Panels show representative transverse clot tissue sections from one healthy donor immunostained for CD15 (A, E), counterstained with Hoechst (B, F), colors merged (C, G), and brightfield (D, H). Images show (A-D) top of fresh clot that was cultured for 30 min at RT (E-G) top of cultured clot that was cultured 4 h at 37° C. Both clots were made from 0.5 mL whole blood cultured in a sterile glass tube with a steel vented cap. Top of clot refers to the portion of the clot in contact with sterile air (fresh clot) or serum (cultured clot). Images were taken in green (1 s) and blue (1 s) channels on a Zeiss Axiovert 200 epifluorescent microscope at 10× magnification, Axiocam 506 monochromatic digital camera and individual channels pseudocolored and combined with czi software. Images were then converted to grey scale. Images are from one representative donor (N=3 total). Scale bars are 100 μm. Dashed line denotes the top edge of the clot.

FIG. 37 shows expression of Lox-1 (37 kDa and sLox-1 (22 & 24 kDa) by HEK293 cells transfected with Lox-1 plasmid DNA and purification by ConA sepharose 4 B affinity beads of recombinant human Lox-1 added to HEK293 conditioned medium, of sLox-1 shed from Lox-1 receptor into conditioned medium of HEK293 cells+pLox-1 transfected cells, and sLox-1 shed into cultured clot serum from 1 human donor. Western blot was probed with anti-extracellular domain antibody to human Lox-1 (AF1798, R&D Systems). rhLox-1 encodes Ser61 to Gln273 and encodes a 29 kDa protein relative to sLox-1 generated by sheddase activity (24 to 28 kDa).

FIG. 38 is a scatterplot showing how coagulopathy during the first 28 days of hospital stay is associated with different degrees of platelet count oscillation as reflected by the standard deviation (SD) of daily platelet counts recorded for each patient enrolled in the ARDS-Omega study. Measures of sLox-1 and IL-8 in blood plasma collected at Day 1 or Day 6 of ICU show sLox-1 is elevated most frequently in patients free of coagulopathy. Coag28 value on the y-axis indicates days free of coagulopathy during 28 days of hospital stay where coagulopathy was defined as platelet count dropping below 80,000 per microliter.

FIG. 39 shows that average daily platelet counts for N=273 patients enrolled in the ARDS-Omega trial are significantly altered when patients are grouped by the explanatory variable 60 day survival with a t Ratio of −5.85 and difference in the mean of −114,000 platelets per microliter for non-survivors (p<0.0001). In a subset of 48 patients in the ARDS-Omega trial, 60 day survival was (B) higher for patients with elevated blood plasma sLox-1 at day 1 or day 6 of mechanical ventilation-assisted blood oxygenation (p=0.038). 60 day survival (C) tends to be lower in N=48 patients with elevated IL-8 at day 1 or day 6 of mechanical ventilation-assisted blood oxygenation (p=0.18).

FIG. 40 shows that chitosan (99% degree of deacetylation, DDA, 3 kDa or 10 kDa) or protamine can be used to reverse heparin anticoagulation and form viable cultured blood clots that express IL8 in the serum after 4 hours at 37° C.

FIG. 41 shows fates of peripheral blood neutrophils in ARDS and their potential effects on lung vasculature.

FIG. 42A shows cultured clot (cC) leukocytes show (A) a common transcriptional shift (B) dominated by Lox-1⁺/CD15⁺ PMN-MDSCs.

FIG. 42B shows cultured clot (cC) leukocytes show decreased FXIIIA/B, increased PR3.

FIG. 42C shows that cultured clot (cC) leukocytes undergoing a transcriptional shift release factors to cC serum with increased sLox-1.

FIG. 43A shows three ARDS survivors had escalating whole blood mRNA expression for (A) OLR1 (B) PRTN3 and (C) platelet factor CXCL7 not seen in (D-F) 3 non-survivors.

FIG. 43B shows ARDS-Omega patients showed (A) high mortality with acute coagulopathy, low platelet oscillation and (B) low platelet counts (p<0.0001), whereas (C) increased sLox-1 was associated with 60-day survival (p<0.05, N=48).

FIG. 44 shows that all cultured clots (with and without LPS or bGP) show (A-B) specific depletion of Factor XIIIA relative to Factor XIIIB and release (A) proteinase 3 (PR3) to the serum, whereas (B) neutrophil elastase (NE) release to serum is donor-specific and inhibited by bGP. FB=fresh blood; FC=fresh clot (30 mM RT incubation); cC=cultured clot (4 h 37° C.; +LPS=cC+100 ng/mL LPS; +bGP=cC+10 mM bGP. This is enabling data for using the cultured clot system to screen drugs for the ability to inhibit neutrophil degranulation in an assay that uses minimally modified whole blood, by measuring the level of NE and/or PR3, or other granule factors (myeloperoxidase, cathepsin G, elastase, metalloproteinases, defensins) in treated versus untreated cultured clot serum.

FIG. 45 shows AAT in fresh plasma (FP), fresh serum (FS), cultured clot serum (cC) and cultured clot serum with LPS (+LPS) or beta-glycerol phosphate (+GP) added is present at about 1000× excess over PR3 +NE in cultured clot serum according to High Resolution Mass Spectrometry proteomics of non-depleted samples from 2 healthy donors.

FIG. 46 demonstrates a novel method for measuring alpha-1 antitrypsin (AAT) deficiency. Panel (A) shows the reaction kinetics of NE combined with buffer-only (samples C1-C4), or diluted serum from 2 different healthy donors (samples 1: fresh serum; 2: cultured clot serum; 3: LPS/cultured clot serum; 4: beta-GP/cultured clot serum), or samples with no NE (bottom row panel A). Panels (B) and (C) show the relative ability for serum AAT to neutralize NE activity, where strong NE inhibition was seen in Donor 1 with fresh serum (arrow), and weakened NE inhibition in cultured clot serum samples that contain proteinase 3 and elastase which consume available AAT.

FIG. 47 shows a portable kit could use miniaturized blood collection tubes, one of which is heated in a small chamber with a battery-driven heating system with a display monitor and temperature sensor/timer. The 2 tubes are color-coded, one for room temperature (normal serum) and one for culture in the heating device. Caps are placed on either end of the glass capillary to avoid evaporation. After the incubation period, liquid expressed from the clot is applied to a lateral flow test with antibodies to the target and a positive control. In another embodiment, the device kit is a phlebotomy kit with 2 red vacutainer tubes. The portable heating device is manufactured so that one vacutainer tube can be inserted inside for 4 hours of incubation at 37 C. The device can optionally be programmed to cool to 4 degrees after 4 hours.

### DETAILED DESCRIPTION OF THE INVENTION

### Definitions and General Techniques

For simplicity and clarity of illustration, the drawing figures illustrate the general manner of construction, and descriptions and details of well-known features and techniques may be omitted to avoid unnecessarily obscuring the present disclosure. Additionally, elements in the drawing figures are not necessarily drawn to scale. For example, the dimensions of some of the elements in the figures may be exaggerated relative to other elements to help improve understanding of embodiments of the present disclosure. The same reference numerals in different figures denote the same elements.

The terms “first,” “second,” “third,” “fourth,” and the like in the description and in the claims, if any, are used for distinguishing between similar elements and not necessarily for describing a particular sequential or chronological order. It is to be understood that the terms so used are interchangeable under appropriate circumstances such that the embodiments described herein are, for example, capable of operation in sequences other than those illustrated or otherwise described herein. Furthermore, the terms “include,” and “have,” and any variations thereof, are intended to cover a non-exclusive inclusion, such that a process, method, system, article, device, or apparatus that comprises a list of elements is not necessarily limited to those elements, but may include other elements not expressly listed or inherent to such process, method, system, article, device, or apparatus.

The terms “left,” “right,” “front,” “back,” “top,” “bottom,” “over,” “under,” and the like in the description and in the claims, if any, are used for descriptive purposes and not necessarily for describing permanent relative positions. It is to be understood that the terms so used are interchangeable under appropriate circumstances such that the embodiments of the apparatus, methods, and/or articles of manufacture described herein are, for example, capable of operation in other orientations than those illustrated or otherwise described herein.

No element, act, or instruction used herein should be construed as critical or essential unless explicitly described as such. Also, as used herein, the articles “a” and “an” are intended to include items, and may be used interchangeably with “one or more.” Furthermore, as used herein, the term “set” is intended to include items (e.g., related items, unrelated items, a combination of related items, and unrelated items, etc.), and may be used interchangeably with “one or more.” Where only one item is intended, the term “one” or similar language is used. Also, as used herein, the terms “has,” “have,” “having,” or the like are intended to be open-ended terms. Further, the phrase “based on” is intended to mean “based, at least in part, on” unless explicitly stated otherwise.

The present invention may be embodied in other specific forms without departing from its spirit or characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

As defined herein, “approximately” can, in some embodiments, mean within plus or minus ten percent of the stated value. In other embodiments, “approximately” can mean within plus or minus five percent of the stated value. In further embodiments, “approximately” can mean within plus or minus three percent of the stated value. In yet other embodiments, “approximately” can mean within plus or minus one percent of the stated value.

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular. Generally, nomenclatures used in connection with, and techniques of, health monitoring described herein are those well-known and commonly used in the art.

The methods and techniques of the present invention are generally performed according to conventional methods well known in the art and as described in various general and more specific references that are cited and discussed throughout the present specification unless otherwise indicated. The nomenclatures used in connection with, and the procedures and techniques of embodiments herein, and other related fields described herein are those well-known and commonly used in the art.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

The percentage given any should be construed based on the weight %, unless indicated otherwise.

The following terms and phrases, unless otherwise indicated, shall be understood to have the following meanings.

As used herein, the terms “subject”, “patient” and “subject in need thereof” may be used interchangeably and refer to a subject in need of administration of the pharmaceutical composition of the invention, or in need of pre-operative, post-operative, or periodic blood sampling to document health status. The term “subject” denotes a mammal, such as a rodent, a feline, a canine, an equine, a goat, a pig, a transgenic pig, a bovine, and a primate, a human or similar.

In an embodiment, subject is involved with production of sLox-1.

In an embodiment, subject could have medical co-morbidities (diabetes, ischemic stroke, systemic lupus erythematosus (SLE), acute respiratory distress syndrome (ARDS), intermittent hypoxia in obstructive sleep apnea syndrome, antitrypsin-1 deficiency, COPD, anti-nuclear cytoplasmic antigen antibodies (ANCA), COVID-19, long-haul COVID-19, cancer, and cardiovascular disease).

In an embodiment, subjects could be healthy subjects.

In an embodiment, subjects are young subjects having age less than 50 years, less than 45, less than 40 or less than 30 years.

As used herein, the term “ex vivo” refers to the process by which cells are removed from a living organism and grown outside the organism (eg, test tube).

As used herein, the term “in vitro” refers to the process by which cells known to grow only in vitro (eg, various cell lines, etc.) are cultured.

Ex vivo production of sLox-1, according to this aspect of the present invention, provides white blood cells (WBC) with conditions for cell growth ex vivo, culturing WBC ex vivo with or without cytokine, thereby allowing production of sLox-1. In another aspect of the present invention, Ex vivo provides neutrophils with conditions for cell growth, culturing neutrophils ex vivo with or without cytokine, thereby allowing production of sLox-1.

As used herein, “culturing” provides the chemical and physical conditions (eg, temperature, gas, etc.) and growth factors required for the maintenance of blood clot. In an embodiment, culturing provides the chemical and physical conditions for maintenance of neutrophils.

As used herein, “cultured blood clot” or “cultured clot” refers to a process when the blood is allowed to clot, for example: a small volume of blood is collected in a sterile container and allowed to clot within an effective duration time to form a clot. In an embodiment, the clot could be collected and put into in a suitable broth to collect serum. In an embodiment, cultured blood clot is allowed to clot for at least about 2 hours.

In an embodiment, “cultured clot” is different from “fresh blood clot.” The fresh blood clot is formed by incubation at room temperature for about 45 minutes and up to as long as standard laboratory tests for patient serum allow.

The advantage of clot culture is that serum can be collected and investigated for serological tests. The cultured blood clot could be either treated or untreated.

As used herein, term “treated cultured blood clot” or “treated cultured clot” refers to when the formation of the blood clot is positively influenced by addition of a drug, inflammatory factor, enhancing factor, coagulant factor, antibody, nanomaterial, nucleic acid, enzyme, pathogen-derived factor etc.

As used herein term, “untreated cultured blood clot” refers to when the formation of the blood clot happens due to its natural process with only the addition of inert anionic surfaces (e.g., glass or silicates) to initiate coagulation.

In an embodiment, cultured clot refers to untreated cultured blood clot unless specifically specified.

As used herein, “cultured clot serum” refers to serum that is collected from a cultured clot. In an embodiment, the cultured clot is centrifuged to collect the serum supernatant free of any cells. The supernatant free of any cell works as a cultured clot serum.

As used herein, the expression “effective duration” of culturing blood clot refers to time required for production of sLox-1. The duration of culturing blood clot suitable for use in some embodiments of the present invention typically ranges from 2 hours to 4 hours to about 5 weeks; varying from 1 hour, 2 hours, 3 hours, 4 hours, 5 hours, 6 hours, 7 hours, 10 hours, 15 hours, 20 hours, 24 hours, 2 days, 3 days, 4 days, 5 days or more.

As used herein, the expression “anti-coagulant factor” refers to an agent or class of agents that prevents coagulation or clotting of blood. For example, but not limited to agents which function by chelating calcium as known in the art. These anticoagulants function by combining with, precipitating and effectively removing calcium ions normally present in the blood. They therefore reduce the concentration of calcium ion in the blood below normal physiological levels. Generally, the anticoagulants which fall within this definition include the citrate anticoagulants, for example, acid citrate dextrose (ACD), citrate phosphate dextrose (CPD), and trisodium citrate (TSC). Ethylenediaminetetraacetic acid (EDTA) may also be used. CPD is the most preferred anticoagulant of the available citrates, as described in U.S. Pat. No. 4,359,463A, which is incorporated herein by reference in its entirety. In an embodiment, heparin is an anticoagulant. In an embodiment, sLox-1 is an anti-coagulant.

As used herein, the term, “coagulation” is also known as clotting, is the process by which blood changes from a liquid to a gel, forming a blood clot. The mechanism of coagulation involves activation, adhesion and aggregation of platelets, as well as deposition and maturation of fibrin.

As used herein, the term, “coagulation enhancing factor or coagulation enhancing material” or “coagulant” like are agents that stimulate the contact pathway of coagulation (membranes containing phosphatidylserine or anionic surfaces such as glass, kaolin, silicates that activate Factor XII) or like agents that respond in a cascade to form fibrin strands, which strengthen the platelet plug. The platelet plug is also known as the hemostatic plug or platelet thrombus, is an aggregation of platelets formed during early stages of hemostasis in response to one or more injuries to blood vessel walls. After platelets are recruited and begin to accumulate around the breakage, their “sticky” nature allows them to adhere to each other. This forms a platelet plug, which prevents more blood from leaving the body as well as any outside contaminants from getting in. The plug provides a temporary blockage of the break in the vasculature. As such, platelet plug formation occurs after vasoconstriction of the blood vessels but before the creation of the fibrin mesh clot, which is the more permanent solution to the injury. The result of the platelet plug formation is the coagulation of blood. For example, but not limited to Tissue factor (TF), a transmembrane glycoprotein in combination of binding factor FVII/FVIIa, lipopolysaccharide (LPS), Cancer procoagulant, etc. In an embodiment, such substances are capable of causing whole blood or a blood component (plasma, platelets) to form a clot.

In an embodiment, a coagulation enhancing material is configured to stimulate coagulation.

In an embodiment, the coagulation enhancing material is configured to reverse anticoagulation.

In an embodiment, the present invention may preferably use calcium ions to reverse the action of the citrate anticoagulant present in plasma feedstock, as described in US20040120942A1, which is incorporated herein by reference in its entirety.

The present invention may preferably use heparinase or protamine to reverse the action of heparin anticoagulant present in plasma feedstock, as described in WO 92/17203 (1992) and Harding et al. (1997), which are incorporated herein by reference in its entirety.

The present invention may preferably use thrombin, or snake venom thrombin-like enzymes (TLE) to reverse the action of citrate anticoagulant present in plasma feedstock, as described in U.S. Pat. No. 6,077,507(A) (2000).

As used herein, the term “blood” used herein means whole blood including hemocytes (erythrocytes, leucocytes, platelets) and plasma (serum) that is a liquid component, and liquid containing at least one of these (for example, blood collected by apheresis). The term “blood” refers to “fresh blood” that is not coagulated. In some embodiments, fresh blood is interchangeably referred as a non-coagulated blood. In an embodiment, blood is free of an external anti-coagulant factor added from outside.

In an embodiment, heparinized anticoagulated blood can be reversed by chitosan to create a cultured blood clot and that this heparin-chitosan cultured clot does release IL8, a biomarker of PMN-MDSCs as shown in FIG. 40, and could therefore also release sLox-1. The degree of deacetylation is preferably from 90% to 100% and the molecular weight is preferably at least 3,000 Da and at most 500,000 Da.

In an embodiment, whole blood is minimally modified. The minimal modification could be less than 10%, 20%, 30% or 40% than the whole blood.

As used herein, the term “serum” means a pale-yellow liquid obtained by allowing collected blood to stand, resulting in reduction of the fluidity, followed by separation from the red coagulated block (clot). As used herein, the term “autologous blood” refers to a patient's own blood. s used herein, the term “homologous blood” refers to that obtained from a blood donor other than the individual for whom the coagulant is prepared.

As used herein, the term, “inflammation enhancing material” or like refers to the material that either leads or increases the inflammatory reaction in mammals. Such materials could be any known to a person skilled in the art. For example, but not limited to Bacterial pathogens such as Lipopolysaccharide, Vasoactive amines, eicosanoids, peptidoglycan, to viral particles, to oxidized low density lipoprotein, and etc.

The term “enhancing material” refers to any material that increases level of sLox-1 in cultured blood clot. For example, but not limited to phorbol esters such as phorbol myristate acetate (PMA), etc. In an embodiment, inflammation enhancing material may work as the enhancing material to increase the level of sLox-1 in cultured blood clot.

As used herein, the term, “viability” of the cells refers to cells not undergoing necrosis or late apoptosis. According to some embodiments, the term “viable cells” refers to cells having an intact plasma membrane. Assays for determining cell viability are known in the art, such as using alamarblue metabolic dye which may be detected in the serum by fluorimetry, or propidium iodide (PI) or calcein AM staining which may be detected by epifluorescence microscopy of the clot or flow cytometry of single cells. Accordingly, according to some embodiments, viable cells are cells which metabolize calcein AM and do not show propidium iodide intake. Necrosis can be further identified, by using light, fluorescence or electron microscopy techniques, or via uptake of the dye trypan blue.

As used herein, the term, “necrosis” of the cells is another mode of cell death as known in the art. A “necrotizing cell disease” includes trauma, ischemia, stroke, myocardial infarction, carbon lethal toxin-induced septic shock, sepsis, LPS-induced cell death and HIV-induced T cell death leading to immunodeficiency, it refers to acute diseases not limited to the above. The term “necrotic cell disease” also refers to chronic neurodegenerative diseases (eg Parkinson's disease, Huntington's disease, amyotrophic lateral sclerosis, Alzheimer's disease, infectious encephalopathy, dementia such as HIV-related dementia). But is not limited.

As used herein, the term, “activating agents” or like is used to provide a surface for reaction. Preferably, the activating agent provides a negatively charged catalytic surface that simulates the contact pathway of coagulation, or by exposure of blood to collagen type I. The intrinsic clotting cascade pathway is initiated by a process called contact activation, a surface and charge dependent phenomenon centered on the activation of Factor XII. Factor XII is highly susceptible to proteolysis because it is bound to surfaces via a charge interaction. The Factor XII precursor has areas of net positive charge that can interact with surfaces with a net negative charge [22]. This charge binding induces conformational changes that enhance the molecule's ability to undergo activation by plasma kallikrein and Factor HK. In an embodiment, activating agent includes thrombin.

Materials commonly used for contact activation are but not limited to borosilicate glass (i.e., hematology glass), silicates, diatomaceous earth, ceramics, ellagic acid and kaolin. Ion exchange resins may also be suitable. Ion exchange resins can provide three separate process' functions in one single material: anionic activation of plasma proteins, a source of calcium to neutralize citrate, and molecular exclusion absorbance to remove water and low molecular weight fluids, thus, concentrating the high molecular weight constituents of the final serum. It has been demonstrated that a borosilicate glass with an anionic surface charge has these preferred features and can be used as an effective activating agent, as described in US20040120942A1 which is incorporated herein by reference it its entirety.

As used herein, the term, “interleukins (IL)” refers to a group of cytokines with complex immunomodulatory functions, including cell proliferation, maturation, migration and adhesion. In an embodiment of the invention, interleukins include human interleukin ILs known to a person skilled in the art is covered according to an embodiment of the invention.

As used herein, the term, “coagulopathy” (also called a bleeding disorder) is a condition in which the blood's ability to coagulate (form clots) is enhanced or impaired. This condition can cause a tendency toward microthrombus formation or platelet depletion followed by prolonged or excessive bleeding (bleeding diathesis), which may occur spontaneously or following an injury or medical and dental procedures. The condition could result from multiple pathological, inheritable, trauma-induced or transfusion induced conditions, causing hypercoagulable or hypocoagulable states that may endanger life. Coagulopathy also occurs following acute trauma and hemorrhage in patients. In an embodiment, coagulopathy refers to platelet level dropped below 80,000 per microliter.

Trauma-induced coagulopathy is often the underlying cause of uncontrolled internal bleeding and, according to some accounts, leads to up to a fivefold increase in patient mortality. In order to normalize blood coagulation condition, a hemostasis therapy is essential that includes transfusion of whole blood or tissue factor concentrates. An early detection of the coagulopathy in patients and monitoring of coagulation metrics during the hemostasis therapy to guide therapeutic endpoints is important.

As used herein, the term, “cardiomyopathy” refers to diseases of the heart muscle. These diseases have many causes, signs and symptoms, and treatments. In cardiomyopathy, the heart muscle becomes enlarged, thick, or rigid. In rare cases, the muscle tissue in the heart is replaced with scar tissue. As cardiomyopathy worsens, the heart becomes weaker. It is less able to pump blood through the body and maintain a normal electrical rhythm. This can lead to heart failure or irregular heartbeats called arrhythmias. In turn, heart failure can cause fluid to build up in the lungs, ankles, feet, legs, or abdomen. The weakening of the heart also can cause other complications, such as heart valve problems.

Main types of cardiomyopathies are dilated cardiomyopathy, hypertrophic cardiomyopathy, restrictive cardiomyopathy, and arrhythmogenic right ventricular dysplasia. Other types of cardiomyopathies sometimes are referred as “unclassified cardiomyopathy.” Cardiomyopathy can be acquired or inherited, with hypertrophic cardiomyopathy and arrhythmogenic right ventricular dysplasia substantially being inherited disorders. In some subjects, inherited cardiomyopathies are not evident until the occurrence of a catastrophic event (e.g., heart attack). Cardiomyopathy can be induced by other diseases or conditions, or by various toxins or drugs. For example, dilated cardiomyopathy can result from coronary heart disease, heart attack, high blood pressure, diabetes, thyroid disease, viral hepatitis, and HIV; infections, especially viral infections that inflame the heart muscle can result in cardiomyopathy; alcohol, especially in conjunction with a poor diet, etc.

As used herein, the term, “LOX-1” or “Lox-1” as used herein is a type II transmembrane cell surface receptor, lectin-like oxidized low density lipoprotein receptor 1, first identified in endothelial cells as one of the main receptors for oxidized-LDL (ox-LDL). Besides ox-LDL, this receptor has been shown to bind many different ligands including other modified lipoproteins, advanced glycosylation end products, aged red blood cells, apoptotic cells, bacteria and activated platelets. Interestingly LOX-1 has been involved in many different pathological conditions including atherogenesis, myocardial ischemia, hypertension, vascular diseases, stroke, lung cancer, COVID-19 and thrombosis [32, 34, 37, 50, 51].

Expression of LOX-1 can be induced by a wide array of stimuli including pro-inflammatory factor (TNF-α, IL-1 or IFN-γ), angiotensin II, endothelin-1, modified lipoproteins and free radicals [32, 52]. Engagement of LOX-1 can lead to induction of oxidative stress, apoptosis, endothelial dysfunction, fibrosis and inflammation through the activation of the NF-κB pathway. LOX-1 has also been described to play a role in tumorigenesis. Indeed, LOX-1 up-regulation has been observed during cellular transformation into cancer cell and can have a pro-oncogenic effect by activating the NF-κB pathway, by increasing DNA damage through increase ROS production and by promoting angiogenesis and cell dissemination. The nucleic acid sequence for the gene encoding LOX-1 (gene name OLR1) can be found in databases such as NCBI, i.e., NCBI gene ID: 4973 or Gene sequence: Ensembl: ENSG00000173391. The LOX-1 protein sequence is found at Hugo Gene Nomenclature Committee 8133, Protein Sequence HPRD:04003.

Term LOX-1 can also represent the receptor protein in various species, and with conservative changes in the amino acid or encoding sequences, or with other naturally occurring modifications that may vary among species and between members of the same species, as well as naturally occurring mutations thereof.

Lox-1 is a scavenger receptor that recognizes oxidized low-density lipoprotein (oxLDL) and activated platelets [29, 53]. In healthy arteries, Lox-1 expression is scarcely detected, but a chronic diet rich in saturated fats and cholesterol induces Lox-1 expression on coronary artery endothelial cells [36]. Endothelial cells exposed to oxLDL upregulate adhesion receptors that capture platelets, and monocytes that can become Lox-1-expressing macrophages, scavenge oxLDL and convert into “foamy” fat-laden cells [53, 54].

oxLDL also stimulates Lox-1-expressing cells to release pro-fibrotic mediators that drive tissue hypertrophy, fibrosis, atrial fibrillation [55].

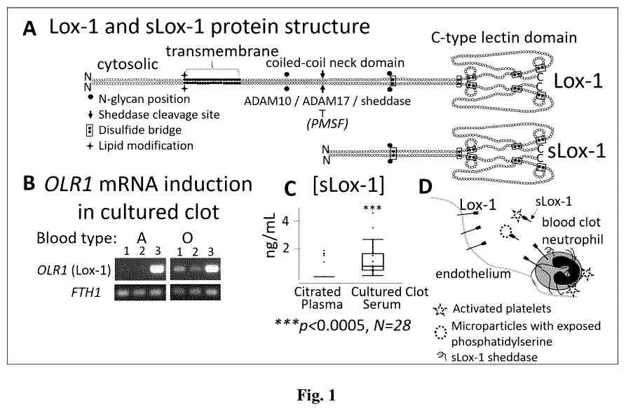

The term, “soluble LOX-1” or “sLox-1” means that a part of LOX-1 existing in the membrane (usually part of “extracellular domain”) is cleaved (dissociated) and released into the blood. Means (shed) part of LOX-1. In an embodiment, sLox-1 is shown in FIG. 1.

Shock trauma after resuscitation from massive hemorrhage stimulates intravascular protease activity (ADAM17) that sheds the glycocalyx from endothelial cells leading to vascular injury [56]. These same proteases were shown to cleave the Lox-1 extracellular domain from this type II transmembrane receptor, to release soluble Lox-1 (sLox-1) (FIG. 1A).

The term “biomarker” as described in this specification includes any physiological molecular form, or modified physiological molecular form, isoform, pro-form, naturally occurring forms or naturally occurring mutated forms of sLOX-1, expressed on the cell surface, unless otherwise specified. Other biomarkers that may be useful to detect cell apoptosis, scramblase activity, flippase activity, ADAM17 activity, ADAM10 activity, alpha secretase activity, proteinase 3 activity, neutrophil elastase activity, Factor XIII activity, sLox-1 sheddase activity, neutrophil secretion or degranulation activity, tumor necrosis factor activation. It is understood that all molecular forms useful in this context are physiological, e.g., naturally occurring in the species. Preferably the peptide fragments obtained from the biomarkers are unique sequences. However, it is understood that other unique fragments may be obtained readily by one of skill in the art in view of the teachings provided herein.

By “isoform” or “multiple molecular form” is meant an alternative expression product or variant of a single gene in a given species, including forms generated by alternative splicing, single nucleotide polymorphisms, alternative promoter usage, alternative translation initiation small genetic differences between alleles of the same gene, and posttranslational modifications (PTMs) of these sequences.

Term, “selective depletion” means a targeted approach towards eliminating unwanted or undesired matter.

Term, “sample” herein refers to blood of a subject. Blood could be whole blood or minimally modified blood as understood by a person skilled in the art that could be used to form a blood clot.

Term, “disease” refers to an abnormal condition that negatively affects the structure or function of all or part of an organism, and that is not immediately due to any external injury.

### Production of sLox-1

In an embodiment, a method of generating, ex vivo production of soluble Lox-1 (sLox-1), comprising introducing a blood sample into a device; adding a coagulation enhancing material in the device before clotting of the blood sample into the device; incubating the device; forming a cultured blood clot in the device; and shedding of the sLox-1 outside the cultured blood clot into the device.

In an embodiment, a method of generating in vitro production of soluble Lox-1 (sLox-1) is provided.

In an embodiment, the blood sample has an anti-coagulant less than 10 wt. %, 9 wt. %, 8 wt. %, 7 wt. %, 6 wt. %, 5 wt. %, 4 wt. %, 3 wt. %, 2 wt. %, 1 wt. %, 0.5 wt. %, 0.1 wt. % or less.

In an embodiment, the blood sample is free from an anti-coagulant factor such as but not limited to heparin.

In an embodiment, the blood sample is non-coagulated blood sample (fresh blood).

In an embodiment, the blood sample may contain coagulating enhancing material to accelerate formation of the clot.

In an embodiment, the blood sample may contain an enhancing material to stimulate sLox-1 production, for example comprises a lipopolysaccharide (LPS), a drug, or blood transfusion products (platelets, liposomes or microparticles), etc.

In an embodiment, the clot is incubated at the temperature around 20° C., 25° C., 30° C., 35° C., 37° C., 40° C., 45° C., 50° C. In an embodiment, the clot is incubated inside the device for about 0.5 hour (hr), 1 hr, 1.5 hrs, 2 hrs, 2.5 hrs, 3 hrs, 3.5 hrs, 4 hrs, 5 hrs, 6 hrs, 7 hrs, 8 hrs, 10 hrs, 12 hrs, 24 hrs or more.

In an embodiment, addition of the enhancing material in the blood forms a treated cultured clot. The cultured clot has about 20%, 30%, 40%, 50%, 60%, 70%, 80%, 90% or more production of sLox-1 compared to an untreated cultured blood clot free of the coagulation enhancing material.

In an embodiment, addition of the enhancing material in the blood forms a treated cultured clot. The cultured clot has about 1×, 2×, 3×, 4×, 5×, 6×, 7×, 8×, 9×, 10× or more production of sLox-1 compared to an untreated cultured blood clot free of the coagulation enhancing material.

In an embodiment, the method is configured to shed sLox-1 more than a fresh blood clot.

In an embodiment, during clot culture, the white blood cells are induced to express OLR1, the mRNA encoding Lox-1, and to shed on average 0.6 ng/mL sLox-1 into the serum. This novel technology enables the investigation of the role of donor sex, age, ethnicity, blood type, and other demographics in mechanisms of Lox-1 induction and sLox-1 shedding.

In an embodiment, the cultured clot system can be a precision medicine tool for detecting the capacity to induce sLox-1 appearance in the serum.

In an embodiment, sLox-1 can be readily and reproducibly generated in a serum sample, by placing whole blood from a donor at 37° C. The high levels of sLox-1 produced after just 4 hours of culture at 37° C. suggests that this approach may be used to generate autologous sLox-1 for clinical use. This approach could also be scalable for larger blood volumes from any species, to produce large amounts of bioactive sLox-1.

In an embodiment, our data shows that the thrombus may be a highly important source of Lox-1 expression and sLox-1 shedding.

In an embodiment, the method is configured to shed 0.5 ng to 50 ng of the sLox-1 per ml of the blood sample.

In an embodiment, the cultured clot serum sLox-1 concentration compared to fresh blood plasma is from 0 pg/mL to 10 ng/mL to 20 ng/mL to 30 ng/mL to 40 ng/mL to 50 ng/mL to 70 ng/mL to 100 ng/mL. In an embodiment, the concentration of sLox-1 in the cultured clot serum compared to fresh blood plasma is about 2 times, 3 times, 4 times, 5 times, 7 times, 10 times, 20 times, 50 times or 100 times more.

Lox-1 binds to phosphatidyl serine and to activated platelets which expose phosphatidyl serine to trigger the contact pathway for thrombin generation. It is therefore feasible that sLox-1 binds to platelet phosphatidyl serine, to have a two-fold effect in muffling the contact pathway activation of thrombin and limiting the ability of platelet microparticles to bind to endothelial cells.

Given these collective data, we postulate that thrombus-induced Lox-1 expression shedding of sLox-1 may be a natural protective mechanism whereby sLox-1 binds platelet microparticles or apoptotic bodies to protect endothelial cells from becoming damaged by these pro-thrombogenic vesicles, and maintain hemostasis after traumatic injury (FIG. 1D).

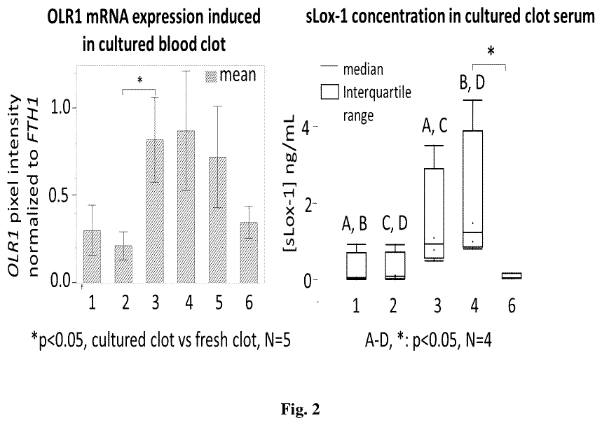

In an embodiment, cultured clots were spiked with LPS by injecting an LPS solution into the vacutainer tube immediately after blood collection and before clot formation. These LPS/cultured clots shed even more sLox-1 into the serum than the paired cultured clots without LPS, in line with the known effect of LPS and downstream mediators (TNF, IL1A) in stimulating Lox-1 expression. (FIG. 2).

In an embodiment, addition of LPS solution forms a treated cultured blood clot.

In an embodiment, in the cultured clot serum and fresh blood, IL-8 and IL-6 concentrations are analyzed along with sLox-1 concentrations.

In an embodiment, the concentration of cultured clot serum IL-8 and/or IL-6 compared to fresh blood plasma is from 50 pg/mL to 10 ng/ml to 20 ng/ml to 40 ng/ml to 50 ng/ml to 70 ng/ml to 100 ng/ml.

In an embodiment, the concentration of cultured clot serum IL-8 compared to fresh blood plasma is about 2 times, 3 times, 4 times, 5 times, 7 times, 10 times, 20 times, 50 times or 100 times more.

In an embodiment, the concentration of IL-6 in cultured clot serum and/or fresh blood plasma is from 0 to 10 ng/ml to 20 ng/ml to 40 ng/ml to 50 ng/ml to 70 ng/ml to 100 ng/mL. In an embodiment, the concentration of cultured clot serum IL-6 compared to fresh blood plasma is about 2 times, 3 times, 4 times, 5 times, 7 times, 10 times, 20 times, 50 times or 100 times more.

In an embodiment, the method is configured to produce an autologous sLox-1. sLox-1 is released from neutrophils.

Lox-1 was reported to be expressed by platelets, however in a different study we refuted this prior work and showed that platelets do not express full-length Lox-1 or OLR1, the mRNA encoding Lox-1. We presented this result in a non-reviewed preprint with SSRN: Leonard, Julia and Hu, Chih-Hsiang and Veneziano, Remi and Hoemann, Caroline D., Investigation of Lox-1 Primary Structure with 6 Anti-Lox-1 Antibodies Suggests that Human Platelets Do Not Express the 37 kDa Lox-1, 24 kDa Soluble Lox-1 or OLR1, the mRNA Encoding Lox-1 (Aug. 16, 2023) [57].

In an embodiment, we carried out quantitative histomorphometry on double immunostained cultured clot cryosections using Lox-1 ICD antibody and anti-CD15, a neutrophil-specific marker [58, 59]. All cells expressing Lox-1 also expressed CD15, suggesting that the sLox-1 is shed from neutrophils (FIG. 35).

In an embodiment, our quantitative histomorphometry results provide satisfactory evidence that neutrophils are the dominant cell type expressing Lox-1 in the cultured clot system (all Lox-1⁺ cells are also CD15⁺). In an embodiment, about 40%, 60%, 80%, 90% or more of all CD15⁺ cells are also Lox-1⁺.

Our new quantitative histomorphometry data revealed that all Lox-1⁺ cells were also CD15⁺, and our correlation data revealed that OLR1 expression is associated with neutrophil counts and not monocyte counts (FIG. 33) indicating that neutrophils are the cell type expressing OLR1, and that on average, 65% of neutrophils in the cultured clot acquired Lox-1 expression after 4 hours of culture. In an embodiment, about 60% 70%, about 80%, about 90% or about 95% or more of neutrophils in the cultured clot acquired Lox-1 expression after 4 hours of culture.

In fresh blood smears, we observe Lox-1⁺/CD15⁻ cells with lymphocyte morphology, but not in any clots.

CD15 is held as a marker that defines human PMN-MDSCs from monocyte MDSCs [60]. Promyelocytes and a small fraction of activated monocytes may express CD15 as detected by flow cytometry, however, it was shown that monocytes express a distinct fucosylase (FUT4) with weak activity and have dull CD15 expression compared to granulocytes which express a highly active fucosylase (FUT9) and strong CD15 expression [58, 59]. Neutrophils outnumber monocytes by 10-fold in normal blood samples. In clot cryosections, all observed Lox-1⁺ cells were also CD15⁺ and therefore deemed to be neutrophils. Due to scarcity of less abundant blood cell types (monocytes or eosinophils) for scoring by histomorphometry, further experiments are needed to determine whether these cell types acquire Lox-1 expression in the cultured clot.

In an embodiment, cultured clot is created with unmodified blood, therefore neutrophil depletion would require additional methodology to permit depletion without activating coagulation. Our quantitative histomorphometry results provide satisfactory evidence that neutrophils are the dominant cell type expressing Lox-1 in the cultured clot system (100% of all Lox-1⁺ cells were CD15⁺).

Among 21 different healthy donors, 4 donors under 30 years old showed unexpectedly high baseline plasma sLox-1 levels (1.1-1.9 ng/mL). In addition, all healthy donors showed a personalized level of clot-induced sLox-1 shedding into cultured clot serum that was reproducible in blood draws from the same donors on different occasions.

Interestingly, fresh clot serum had about 1 ng/mL or more, less sLox1 than FB (FB: fresh blood) plasma, in donors with baseline sLox-1 (p<0.05, N=4). These results, along with the lack of sLox-1 in fresh clot serum from the other donors, ruled out the possibility that activated platelets were releasing sLox-1.

In an embodiment, histomorphometric analyses of clots from 3 donors suggested that all Lox-1 expressing cells were neutrophils. In double immunostained clot sections, all Lox-1⁺ cells in fresh clots and cultured clots also expressed CD15, one of the minimally accepted markers of PMN-MDSCs [60]. The percentage of Lox-1⁺/CD15⁺ cells increased from 28% in fresh clots to 46% in cultured clots (p<0.05, N=3 donors). Around 1% of CD15⁺ cells appeared to form a synapse with a lymphocyte.

In an embodiment, neutrophils respond to activated platelets by forming neutrophil platelet aggregates (NPA) [61, 62]. In an embodiment, ADAM17 requires phosphatidylserine exposure for sheddase function [47] suggests that sLox-1 may be shed from neutrophil-platelet aggregates (NPA) or apoptotic neutrophils exposed to thrombin.

Antithrombin, thrombomodulin, and its effector activated protein C were all trialed as ARDS therapeutics but did not show meaningful clinical effects [1, 63, 64]. PMN-MDSCs arise in disease states marked by hypoxia including lung cancer, sepsis, intermittent obstructive sleep apnea, COVID and ARDS [37, 50, 65-67]. PMN-MDSCs are implicated in both infection and cancer for immune suppression of T cells through ROS generation.

Lectin-like oxidized LDL receptor-1 (Lox-1) was identified as a specific marker of CD15⁺ PMN-MDSCs in a study of circulating neutrophils in lung cancer [37]. Lox-1 was originally identified as an endothelial scavenger receptor for oxidized LDL (oxLDL) in the context of atherosclerosis [29].

OLR1, the mRNA encoding Lox-1, was afterwards found in a variety of other cell types including alveolar macrophages, alveolar epithelium and endothelium, vascular smooth muscle, synoviocytes, and activated macrophages [68-71]. Endothelial cell and macrophage Lox-1 expression is amplified by inflammatory factors including tumor necrosis factor (TNF), oxLDL and phorbol myristate acetate (PMA) [72]. Upon activation, Lox-1 deploys inflammatory mediators [34] that can amplify lung damage in ARDS.

In an embodiment, fresh clots and cultured heparin blood had similar OLR1 expression levels as FB.

In an embodiment, the present invention unravels the mechanisms of how PMN-MDSCs generate sLox-1.

In an embodiment, we take a step in a new direction towards identifying PMN-MDSCs as a beneficial cell type to arise in ARDS, which could lead to identifying new strategies to protect platelet levels and endothelial barrier function.

In an embodiment, variability in OLR1 induction in cultured clots could be potentially due to different neutrophil counts and susceptibility of neutrophils to apoptosis or senescence in cultured clots.

Data in this study are important because they suggest a new mechanism for mature peripheral blood neutrophils to undergo rapid PMN-MDSC polarization and raise the possibility that sLox-1 is shed from neutrophils in pro-thrombotic disease states including COVID-19 and potentially ARDS [45], as shown in FIGS. 38 and 39.

In an embodiment, neutrophil suppressor cells emerge in pro-coagulant environments to release factors that safeguard the vascular endothelium. This premise is supported by work in our lab using a simple in vitro cultured whole blood clot system. Cultured clot neutrophils were found to rapidly polarize to suppressor cells and release soluble Lox-1 (sLox-1).

In an embodiment, cultured clot serum showed potent endothelial barrier enhancing activity. Mass Spectrometry proteomics of cultured clot serum has provided an additional clue that proteinase 3, a neutrophil granule enzyme, could be a sLox-1 sheddase.

In an embodiment, we investigate mechanisms inducing peripheral blood neutrophils to produce sLox-1 in vitro.

In an embodiment, we are using tissue-engineered blood clots as ex vivo models of thrombosis [73]. We discovered that neutrophils in cultured clots from healthy donors skew to a suppressor cell signature and shed sLox-1 to the serum. Table 1 provides donor demographics for RNA sequencing study.

TABLE 1
Baseline characteristics in healthy blood donors (N = 10)

| | |
|---|---|
| Feeling healthy | yes |
| Age, y | 30 (20-60) |
| (minimum 18 yrs old) | |
| Covid status | Negative PCR test (N = 10) |
| Sex | 4 Female, 6 Male |
| Body Mass Index | 24.5 (21.5-28.8) |
| (BMI), kg/m | |
| White Blood Count | 7.1 (4.8-13.2) |
| (WBC) × 10\hat{ }6/L | |
| Ethnicity | Caucasian, Asian, Hispanic/Latin, African |
| | America |
| ABO blood types | A, B, O |
| Rh-factor | Rh-positive, Rh-negative |
| Lewis blood alleles | Null, Lea, Leb, Lea/Leb |
| Duffy blood alleles | Null, Fyb, Fya/Fyb, Fya (nonsecretor) |
| Alcohol consumption | 0.6 (0-2) |
| 0 = none, 1 = 1-3/wk, | |
| 2 = 3+/wk | |
| Smoking (0 = never, 1 = | 0.5 (0-2) |
| former, 2 = yes) | |
| Allergies (0 = none, 1 = yes) | 0.3 (0-1) |
| Pain medication | None |

In fresh blood smears of healthy donors, CD15⁺ neutrophils showed only background staining for Lox-1, but surprisingly, strong Lox-1 staining was detected in cells with a lymphocyte morphology in FB smears but not in cultured clots. By contrast, in fresh and cultured clots, all cells expressing Lox-1 also expressed CD15 (FIG. 35A-D). Quantitative histomorphometry showed that the percentage of CD15⁺ cells expressing Lox-1 increased from 36%±20% in fresh clots to 65%±7% of CD15⁺ cells in cultured clots (p<0.05, N=3 donors, FIG. 35E), with only minor co-localization of the two receptors. Lox-1⁺/CD15⁺ neutrophils were also detected using an anti-Lox-1 extracellular domain antibody however with strong background staining in the clot structure.

Lox-1⁺/CD15⁺ neutrophils tended to cluster along the surface of the cultured clot and in platelet-rich fibrin areas (FIG. 36). Around 1% of CD15⁺ clot neutrophils formed a tight association with a lymphocyte in 4 different healthy donors. Interestingly, Lox-1⁺/CD15⁺ neutrophils were detected in a blood smear collected from one donor 2 weeks post-COVID-19 infection and no longer detected 3 months later.

In an embodiment, sLox-1 has all the hallmarks of a resolvin, because it is a decoy receptor for Lox-1, a pro-inflammatory endothelial receptor for many pro-coagulant factors (FIG. 41).

In an embodiment, serum collected from cultured blood clots of healthy donors exhibited an exceptionally strong capacity to enhance endothelial barrier function in vitro. High-Resolution Mass spectrometry (MS) revealed the presence of proteinase 3 (PR3) within this serum.

In an embodiment, extracellular PR3, which co-localizes with Lox-1 to lipid rafts, serves as a Lox-1 sheddase. sLox-1 appearance in cultured clot serum but not fresh serum (FIGS. 13B and 23) was accompanied by PR3 appearance in cultured clot serum but not fresh serum (FIG. 44). In a pilot study of 48 ARDS-Omega blood plasma biospecimens, sLox-1 was associated with higher 60 day survival.

In an embodiment, plasma sLox-1 is positively associated with 60-day survival in ARDS patients and activated platelets are sufficient to stimulate blood neutrophils to produce sLox-1.

In normal healthy individuals neutrophil counts may vary widely, from 1.5 to 7.0 millon per mL. Although several of our healthy donors had over 7.0 million neutrophils per mL, neutrophil counts alone did not explain differences in baseline OLR1 expression. Considering our quantitative histomorphometry data showing that, on average, 65% of cultured clot neutrophils acquired Lox-1 expression in 3 healthy donors, we believe that at least some variation in cultured clot OLR1 expression and sLox-1 production is likely to be related to absolute neutrophil counts.

In an embodiment, variability in OLR1 induction in cultured clots could be potentially due to different neutrophil counts (FIG. 33) and susceptibility of neutrophils to apoptosis or senescence in cultured clots.

Although the biological basis for differential OLR1 and sLox-1 expression between donors remains to be fully elucidated, if sLox-1 serves a protective role, then individuals with a more robust sLox-1 innate immune response may have a better prognosis for certain disease contexts.

In an embodiment, number of CD15⁺ cells is similar in fresh blood and cultured clot (4 hours 37° C.). The difference is that more cultured clot neutrophils are Lox-1⁺/CD15⁺ than the fresh clot.

In an embodiment, method is configured to estimate an amount of ACTIVE alpha-1 antitrypsin in a sample to detect a disease in a subject. The inclusion of the term “activity” is important because alpha-1 antitrypsin (AAT) deficiency can occur due to inactivating mutations in the protein that is still very abundant in the blood stream. AAT is an inhibitory protein released from the liver and present at highly abundant levels in blood plasma. It forms an inactive complex with proteinase 3 and elastase.

In an embodiment, we unexpectedly detected abundant levels of proteinase3/elastase in cultured clot serum and not in fresh serum, and also unexpectedly, we were unable to measure any proteinase3/elastase enzyme activity in cultured clot serum samples, presumably due to the presence of normal active levels of alpha-1 antitrypsin in the healthy donor blood sample.

In an embodiment, unexpected finding that cultured clot serum, despite containing high levels of proteinase 3 and elastase protein (i.e., neutrophil granule enzymes that are not present in fresh clot serum, Mass Spectrometry data), is devoid of proteinase 3/elastase activity (presumably due to presence of inhibitory levels of alpha-1 antitrypsin).

These findings suggest a new way to screen for personalized alpha-1 antitrypsin deficiency, namely, generate fresh serum and cultured clot serum from a patient, and then see whether it is possible to detect proteinase 3 or elastase activity in the cultured clot serum. Detection of even minute amounts of proteinase3/elastase activity would suggest functional AAT deficiency, higher risk factors if the patient develops pneumonia, and the potential for chronic lung damage that could eventually lead to COPD.

Another embodiment would be to collect fresh serum from a patient, and then add increasing amounts of purified proteinase 3 or elastase, and then measure the inhibitory activity of the patient serum towards these enzymes that can diminish endothelial barrier function.

In an embodiment, we could also carry out an assay to measure the inhibitory potential of the clot serum towards neutrophil elastase.

### Diagnostic Application

sLox-1 is a soluble glycoprotein homodimer released by ectodomain shedding of (Lox-1) [55]. Lox-1 and sLox-1 both contain the same binding sites for ligands with pro-coagulant activity: oxLDL, phosphatidylserine (PS) and activated platelets [74-76]. As a decoy receptor, sLox-1 could provide protection against endothelial-damaging effects of oxLDL and also against the pro-coagulant activity of PS-bearing extracellular vesicles [5].

In an embodiment, the present invention identifies plasma sLox-1 is a new prognostic biomarker of 60-day survival in ARDS. In another embodiment, biomarker could be identified less than 60 days such as 10 days, 20 days, 30 days, 50 days etc. In another embodiment, biomarker could be identified for more than 60 days such as 70, 80, 90, 100 days and so on.

In an embodiment, we identified that blood plasma sLox-1 was associated with 60-day survival in a pilot study of 48 specimens from the ARDS-Omega trial. Future studies will determine whether sLox-1 is a novel prognostic biomarker of survival in ARDS using BioLINCC specimens from the ARDS network (Omega, SAILS, days 0, 3 and 6; and ALTA) and healthy controls (DASH).

In an embodiment, the cultured clot system is used as a novel diagnostic blood test that mimics a thrombus. The ex vivo thrombus device was optimized with blood samples from a cohort of 37 consenting healthy donors with diverse demographics (male/female, 18-61 years old, smoking or non-smoking; Caucasian/Hispanic/Latino/Asian/African-American; blood types ABO, Rh+/−, Lewis a/b/null, Duffy a/b/null, secretor/non-secretor).

sLox-1 is proposed as a biomarker of early-stage acute coronary syndrome and myocardial infarction. In specific disease states (lung cancer, thrombotic influenza, thrombotic COVID), peripheral blood neutrophils can acquire Lox-1 expression [37, 50, 67].

In an embodiment, identifying PMN-MDSCs as a beneficial cell type to arise in ARDS that could lead to identifying new strategies to protect platelet levels and endothelial barrier function.

In an embodiment, sLox-1 as a new prognostic biomarker associated with survival in acute respiratory distress syndrome (ARDS). We have gathered compelling evidence to suggest that neutrophil myeloid-derived suppressor cells could play a beneficial role in ARDS by releasing mediators that safeguard the endothelial barrier. Mechanistic studies will identify factors that cause neutrophils to shed sLox-1 which could lead to new strategies to alleviate immunothrombosis in ARDS.

In an embodiment, circulating neutrophils exposed to activated platelets shift to a PMN-MDSC phenotype and release mediators (i.e. sLox-1) that suppress thrombosis and promote survival.

In an embodiment, a role for EP4 receptor on endothelial cells as mediating the protective effect [77] which further suggested that cultured clot serum contains oxidized lipids that are mediating the protective effect.

TABLE 2
ARDS studies of PMN-MDSCs and Lox-1/sLox-1 as related to reduced mortality.

| | | | | | Biomarker & |
|---|---|---|---|---|---|
| STUDY | ARDS Cohorte | Biomarker | Coagulopathy | % mortality | survival [symbol] |
| Wang 2014 | Beijing cohort, ARDS | [symbol] platelets | 26% ARDS | 31% ARDS | [symbol] platelets |
| | N = 75, cntl N = 103 | | 26% cntl | 38% cntl | [symbol] mortality D60 |
| Schulte-Schr- | COVID-19, mild, severe | [symbol] MDSC | NR | NR | NR |
| epping 2020 | N = 35 | [symbol] severity | | | |
| Combadière | COVID-19 ICU vs non- | [symbol] Lox-1+ PMN | 42% ICU | 21% ICU | NR |
| 2021 | ICU (no M.V.) N = 38 | [symbol] [symbol] | 7% Non-ICU | 0% Non-ICU | |
| Sacci 2021 | SARS-COV-2+, N = 62 | [symbol] PMN-MDSC | NR | NR | NR |
| Coudereau | COVID-19 N = 39 septic | [symbol] Lox-1+ | NR | 8% (day 28) | NR |
| 2022 | shock N = 48 | PMN-MDSC | | | |
| Korkmaz | Pneumonia ARDS | [symbol] sLox-1 | NR | NR | NR |
| 2022 | N = 11 Healthy Cntl N = 9 | BALF | | | |
| Our metadata | Bacterial and viral | OLR1 | NR | 3 died | [symbol] OLR1 in 3 |
| (Parnell 2013) | sepsis ARDS (N = 10) | | | 7 survived | survivors (D5) |
| Our pilot data | [symbol] platelet oscillation | sLox-1 | 40% yes | 35% (N = 48) | [symbol] sLox-1 |
| Omega | [symbol] coagulopathy | (plasma) | 60% no | Day 60 | [symbol] 85% survival |
| (N = 48) | | | | | |

Symbols: NR: Not reported [symbol] [symbol] [symbol] elevated [symbol] [symbol] reduced; ICU: intensive care unit; D60: day 60 [symbol] cntl: non-ARDS. [symbol]
indicates data missing or illegible when filed

The RNAseq study revealed that OLR1, the mRNA encoding Lox-1, is significantly induced in the ex vivo thrombus relative to fresh blood and fresh blood clots (FIG. 1B), along with the gene signature for MDSCs (CXCL8, SSP1, CXCL2, CCL2, IL1A, TNF, VEGFA). Cultured clot histology showed Lox-1-expressing clot neutrophils.

In an embodiment, proteinase 3 becomes elevated in blood plasma during thrombogenic viral infection.

In an embodiment, the bulk RNAseq experiment revealed 1,546 differentially expressed genes in cultured clots relative to fresh blood, and among these, biostatistical analyses revealed a cluster of genes that contained many markers of PMN-MDSCs, including OLR1.

In an embodiment, we analyzed more genes than OLR1 in our study, including a panel of PMN-MDSC markers by RT-PCR (VEGFA, SPP1, CXCL2, IL1A TREM1), as well as IL-8 and IL-6 by proteomics in addition to sLox-1.

In an embodiment, we chose to study OLR1 in more depth because it has been identified as a specific but controversial marker that is believed to distinguish PMN-MDSCs from classical PMN (Condamine T, Dominguez GA, Youn J-I, Kossenkov AV, Mony S, Alicea-Torres K, et al. Lectin-Type Oxidized LDL Receptor-1 Distinguishes Population of Human Polymorphonuclear Myeloid-Derived Suppressor Cells in Cancer Patients. Sci Immunol (2016) 1(2):aaf8943. doi: 10.1126/sciimmunol.aaf 8943) [37].

The finding that circulating neutrophils in COVID-19 acquire Lox-1 expression increased our interest in studying this marker. In addition to our discovery that OLR1/Lox-1 expression is induced in the thrombus itself, circulating PMN-MDSCs are a population of cells of great interest in the fields of cancer and infection and we believe that the focus on OLR1 expression in our work will increase the relevance of our findings for a broader audience.

During the course of this study, we examined Lox-1, CD15, and Lox-1/CD15 double immunostained samples that included 7 fresh blood smear samples (5 donors and 2 technical replicates), and 5 fresh clot samples and 5 cultured clot samples from 4 distinct healthy donors with 1 technical replicate. Methods: Clots were fixed in formalin, infiltrated with sucrose and Tissue-Tek, and 8 μm thick cryosections collected on scotch tape and stored at −20° C. (N=4 donors). EDTA whole blood smears were stored frozen at −80° C., then thawed and fixed in NBF (N=5 donors).

Several groups have used Lox-1 cell surface expression by flow cytometry along with CD15 to demonstrate the presence of peripheral blood PMN-MDSCs in patients with cancer or COVID-19. In addition to COVID-19 lung infection, it was recently reported that tuberculosis lung infection elicits peripheral blood neutrophils expressing OLR1 and other PMN-MDSC markers.

Immunosuppressive genes previously detected as upregulated in human PMN-MDSCs include NOS2, Arg1 , IL10, ADAM17, CD274/PD-L1, and Lgals9 [78-82]. Of these genes, IL10 (LFC>2.4) and ADAM17 (LFC>0.59) were upregulated in cultured clot, with or without LPS compared to FB (p.adj≤0.012) and were not induced in cultured heparin blood.

In an embodiment, sLox-1 becomes elevated in blood plasma during thrombogenic viral infection.

In an embodiment, sLox-1 is a biomarker.

In an embodiment, present invention helps to detect cell apoptosis, scramblase activity, flippase activity, ADAM17 activity, ADAM10 activity, alpha secretase activity, degranulation activity, elastase or proteinase 3 activity, neutrophil-platelet aggregation activity, sLox-1 sheddase activity, tumor necrosis factor activation.

In an embodiment, our novel data show that release of sLox-1 from the cultured clot is associated with those cells (neutrophils) that have a profile of PMN-MDSCs, which are known to drive cancer progression and inhibit immune defense against infection.

“Gene Ontology” pathways induced in the cultured clot samples from 10 different (diverse) human donors that are induced by factors that are released from platelets (Transforming Growth Factor Beta). We also see pathways induced in the cultured clots from 5 different donors treated with lipopolysaccharide—that are activated by lipopolysaccharide, so the system is definitely working.

The new enabling data in FIGS. 42 and 44 provide measuring proteinase 3 protein levels or proteinase 3 activity, or elastase protein levels or elastase activity, as a method to determine alpha 1 antitrypsin deficiency using the cultured clot system. In an embodiment, a functional diagnostic test for AAT deficiency using the cultured clot test. It could be used as a screening tool to determine whether neutrophil degranulation in the blood stream could lead to endothelial barrier damage.

Table 3 shows GO pathways upregulated and downregulated in the cultured clotted blood.

In an embodiment, cultured clot device could be used to screen natural products derived from a host of microbial sources, yeast, bacteria, and screen for donor-specific inflammatory activity.

In FIG. 32, GO pathway analysis of the top 10 pathways upregulated in (A) cultured clots vs FB, (B) LPS/cultured clots vs FB (C) LPS/cultured clots vs cultured clots. Panel D) DEG analysis of LPS/cultured clot and cultured clot vs. FB. Cluster analysis by direction of Log2-fold change of conditions LPS/cultured clot vs FB (N=5) and cultured clot vs FB (N=10) showed that all cultured clots upregulated genes associated with PMN-MDSCs (++cluster), downregulated markers of platelets (−−cluster), and showed LPS-induced changes in pro-inflammatory mediators (+o, −o clusters). Panel E) Average QN counts of genes involved in MDSC activation (IL10, SPP1, OLR1, IL-8) and thrombosis (F3, PF4) for FB (N=10, LPS/cultured clot (N=5), cultured clot (N=10), cultured heparin blood (N=2), LPS/cultured heparin (N=2). GO pathways involved in vascular development (2.85e-7) and angiogenesis (p=1.82e-5) were also upregulated in cultured clots. Loss of platelet-specific mRNA read counts in cultured clots is evidence to suggest that platelet mRNA becomes labile after platelet activation in the clot.

In an embodiment, the cultured clot system can be a precision medicine tool for detecting the capacity to induce proteinase 3 (PR3) or PR3 activity in the serum, or neutrophil elastase (NE) or NE activity in the serum.

In an embodiment, the cultured clot system can be a precision medicine tool for detecting the selective depletion of Factor XIIIA compared to Factor XIIIB in the plasma or serum.

TABLE 3
GO pathways upregulated and downregulated in the cultured clotted blood samples.

| | GO Term; UPREGULATED: | | | | | | |
|---|---|---|---|---|---|---|---|
| | CULTURED CLOT VS FRESH BLOOD | Ont | N | Up | Down | P. Up | P. Down |
| GO: 0072599 | SRP-dependent cotranslational protein | BP | 107 | 71 | 5 | 4.11418E−10 | 1 |
| | targeting to membrane | | | | | | |
| GO: 0006613 | cotranslational protein targeting to | BP | 94 | 64 | 3 | 6.95707E−10 | 1 |
| | membrane | | | | | | |
| GO: 0045047 | protein targeting to ER | BP | 104 | 68 | 5 | 2.7585E−09 | 1 |
| GO: 0016070 | RNA metabolic process | BP | 2417 | 985 | 679 | 7.6843E−09 | 1 |
| GO: 0032502 | developmental process | BP | 2593 | 1045 | 835 | 3.22322E−08 | 0.997810545 |
| GO: 0022626 | cytosolic ribosome | CC | 91 | 59 | 7 | 5.28897E−08 | 1 |
| GO: 0030154 | cell differentiation | BP | 1693 | 703 | 540 | 7.24897E−08 | 0.992740251 |
| GO: 0009889 | regulation of biosynthetic process | BP | 2102 | 855 | 610 | 1.39456E−07 | 0.999999999 |
| GO: 0048869 | cellular developmental process | BP | 1732 | 715 | 552 | 1.58995E−07 | 0.993869456 |
| GO: 0070848 | response to growth factor | BP | 312 | 155 | 92 | 2.36224E−07 | 0.973011798 |
| GO: 0001944 | vasculature development | BP | 313 | 155 | 93 | 2.8547E−07 | 0.966537216 |
| GO: 0071363 | cellular response to growth factor stimulus | BP | 301 | 149 | 89 | 5.03828E−07 | 0.968574853 |
| GO: 0009888 | tissue development | BP | 708 | 315 | 224 | 5.25253E−07 | 0.951952544 |
| GO: 0072359 | circulatory system development | BP | 444 | 208 | 128 | 5.79882E−07 | 0.995232251 |
| GO: 0048514 | blood vessel morphogenesis | BP | 258 | 130 | 80 | 7.64085E−07 | 0.888221411 |
| GO: 0048856 | anatomical structure development | BP | 2381 | 956 | 772 | 8.68121E−07 | 0.991967192 |
| GO: 0050793 | regulation of developmental process | BP | 1114 | 474 | 354 | 8.8679E−07 | 0.977298487 |
| GO: 0007275 | multicellular organism development | BP | 2160 | 872 | 692 | 1.42986E−06 | 0.996508816 |
| GO: 0006612 | protein targeting to membrane | BP | 154 | 85 | 24 | 1.81398E−06 | 0.999999978 |
| GO: 1990830 | cellular response to leukemia inhibitory | BP | 45 | 31 | 8 | 7.99242E−06 | 0.995729675 |
| | factor | | | | | | |
| GO: 0051171 | regulation of nitrogen compound metabolic | BP | 2840 | 1117 | 867 | 1.27987E−05 | 0.999999963 |
| | process | | | | | | |
| GO: 0035295 | tube development | BP | 402 | 186 | 128 | 1.29135E−05 | 0.871345728 |
| GO: 0004843 | thiol-dependent ubiquitin-specific protease | MF | 58 | 37 | 9 | 1.60005E−05 | 0.999614433 |
| | activity | | | | | | |
| GO: 0001525 | angiogenesis | BP | 230 | 114 | 75 | 1.82277E−05 | 0.736412159 |
| GO: 0043484 | regulation of RNA splicing | BP | 114 | 62 | 23 | 5.37545E−05 | 0.99972374 |
| GO: 0006807 | nitrogen compound metabolic process | BP | 4813 | 1828 | 1540 | 6.99652E−05 | 0.999999981 |
| GO: 0043066 | negative regulation of apoptotic process | BP | 455 | 204 | 132 | 8.17019E−05 | 0.99539686 |
| GO: 0030509 | BMP signaling pathway | BP | 49 | 31 | 7 | 9.50506E−05 | 0.999558086 |
| GO: 0071559 | response to transforming growth factor beta | BP | 112 | 59 | 32 | 0.000243179 | 0.918663089 |

| | Go Term: DOWNREGULATED | | | | | | |
|---|---|---|---|---|---|---|---|
| | CULTURED CLOT VS FRESH BLOOD | Ont | N | Up | Down | P. Up | P. Down |
| GO: 0002275 | myeloid cell activation involved in immune | BP | 453 | 108 | 227 | 0.999999999 | 9.15019E−13 |
| | response | | | | | | |
| GO: 0043299 | leukocyte degranulation | BP | 446 | 106 | 224 | 0.999999999 | 9.78558E−13 |
| GO: 0002444 | myeloid leukocyte mediated immunity | BP | 458 | 112 | 227 | 0.999999994 | 3.63867E−12 |
| GO: 0036230 | granulocyte activation | BP | 429 | 103 | 215 | 0.999999995 | 3.69916E−12 |
| GO: 0045055 | regulated exocytosis | BP | 550 | 140 | 264 | 0.999999995 | 4.95593E−12 |
| GO: 0042119 | neutrophil activation | BP | 424 | 101 | 212 | 0.999999996 | 6.8324E−12 |
| GO: 0002443 | leukocyte mediated immunity | BP | 608 | 162 | 287 | 0.999999966 | 7.24162E−12 |
| GO: 0071944 | cell periphery | CC | 2172 | 776 | 870 | 0.747102008 | 2.02024E−11 |
| GO: 0002283 | neutrophil activation involved in immune | BP | 417 | 99 | 207 | 0.999999996 | 2.78222E−11 |
| | response | | | | | | |
| GO: 0043312 | neutrophil degranulation | BP | 415 | 99 | 206 | 0.999999994 | 3.14096E−11 |
| GO: 0002446 | neutrophil mediated immunity | BP | 424 | 102 | 209 | 0.999999993 | 4.99264E−11 |
| GO: 0006955 | immune response | BP | 1349 | 422 | 565 | 0.999993741 | 1.43594E−10 |
| GO: 0002366 | leukocyte activation involved in immune | BP | 570 | 159 | 266 | 0.999996906 | 1.61592E−10 |
| | response | | | | | | |
| GO: 0005886 | plasma membrane | CC | 2049 | 731 | 819 | 0.756401928 | 1.73932E−10 |
| GO: 0002263 | cell activation involved in immune | BP | 573 | 159 | 267 | 0.999997984 | 1.78465E−10 |
| | response | | | | | | |
| GO: 0006887 | exocytosis | BP | 622 | 166 | 286 | 0.999999967 | 2.10103E−10 |
| GO: 0005766 | primary lysosome | CC | 134 | 22 | 81 | 0.999999927 | 6.02838E−10 |
| GO: 0031982 | vesicle | CC | 2142 | 714 | 847 | 0.999725127 | 1.72896E−09 |
| GO: 0034774 | secretory granule lumen | CC | 234 | 50 | 124 | 0.99999988 | 2.30658E−09 |
| GO: 0002252 | immune effector process | BP | 897 | 259 | 387 | 0.999999825 | 2.86053E−09 |
| GO: 0035578 | azurophil granule lumen | CC | 75 | 7 | 51 | 0.999999988 | 3.41284E−09 |
| GO: 0043230 | extracellular organelle | CC | 1189 | 389 | 494 | 0.998446117 | 1.05717E−08 |
| GO: 0099503 | secretory vesicle | CC | 594 | 168 | 267 | 0.99999468 | 1.19739E−08 |
| GO: 0045321 | leukocyte activation | BP | 911 | 293 | 388 | 0.998002764 | 1.48654E−08 |
| GO: 1903561 | extracellular vesicle | CC | 1187 | 389 | 492 | 0.998192204 | 1.62932E−08 |
| GO: 0001775 | cell activation | BP | 986 | 319 | 416 | 0.997943178 | 1.63864E−08 |
| GO: 0070062 | extracellular exosome | CC | 1181 | 388 | 489 | 0.997735197 | 2.13583E−08 |
| GO: 0016491 | oxidoreductase activity | MF | 341 | 91 | 162 | 0.999968748 | 1.65727E−07 |
| GO: 0005759 | mitochondrial matrix | CC | 242 | 52 | 121 | 0.999999907 | 2.25406E−07 |

| | GO Term: UPREGULATED | | | | | | |
|---|---|---|---|---|---|---|---|
| | LPS/cultured clot vs cultured clot | Ont | N | Up | Down | P. Up | P. Down |
| GO: 0006952 | defense response | BP | 952 | 353 | 185 | 1.33836E−43 | 0.999664101 |
| GO: 0034097 | response to cytokine | BP | 747 | 298 | 122 | 2.85206E−43 | 0.999999924 |
| GO: 0019221 | cytokine-mediated signaling pathway | BP | 514 | 229 | 80 | 3.96671E−42 | 0.999998982 |
| GO: 0043207 | response to external biotic stimulus | BP | 879 | 329 | 159 | 1.87029E−41 | 0.999990667 |
| GO: 0051707 | response to other organism | BP | 879 | 329 | 159 | 1.87029E−41 | 0.999990667 |
| GO: 0006955 | immune response | BP | 1349 | 432 | 316 | 1.61044E−35 | 0.615028294 |
| GO: 0045087 | innate immune response | BP | 582 | 223 | 102 | 3.07093E−29 | 0.999921308 |
| GO: 0034341 | response to interferon-gamma | BP | 140 | 83 | 14 | 4.08204E−26 | 0.999991177 |
| GO: 0006950 | response to stress | BP | 2179 | 586 | 473 | 4.04312E−25 | 0.996182236 |
| GO: 0009615 | response to virus | BP | 251 | 119 | 28 | 7.02835E−25 | 0.999999908 |
| GO: 0006954 | inflammatory response | BP | 405 | 165 | 92 | 7.13008E−25 | 0.708216575 |
| GO: 0044419 | biological process involved in interspecies | BP | 1398 | 409 | 232 | 3.15439E−24 | 1 |
| | interaction between organisms | | | | | | |
| GO: 0071346 | cellular response to interferon-gamma | BP | 125 | 74 | 13 | 2.36E−23 | 0.999958163 |
| GO: 0031347 | regulation of defense response | BP | 414 | 163 | 68 | 9.83303E−23 | 0.999921368 |
| GO: 0001816 | cytokine production | BP | 486 | 183 | 105 | 1.09268E−22 | 0.890051916 |
| GO: 0009617 | response to bacterium | BP | 339 | 138 | 70 | 4.00329E−21 | 0.916260383 |
| GO: 0002682 | regulation of immune system process | BP | 958 | 297 | 202 | 4.99431E−21 | 0.981901904 |
| GO: 0032496 | response to lipopolysaccharide | BP | 204 | 93 | 42 | 2.8311E−18 | 0.87365883 |
| GO: 0060333 | interferon-gamma-mediated signaling | BP | 73 | 47 | 7 | 2.72406E−17 | 0.999482311 |
| | pathway | | | | | | |
| GO: 0060337 | type I interferon signaling pathway | BP | 72 | 46 | 6 | 1.02842E−16 | 0.999838461 |
| GO: 0007154 | cell communication | BP | 2892 | 691 | 734 | 2.16664E−15 | 0.005293075 |
| GO: 0071356 | cellular response to tumor necrosis factor | BP | 183 | 79 | 26 | 3.83497E−14 | 0.999522027 |
| GO: 0045321 | leukocyte activation | BP | 911 | 261 | 239 | 1.06492E−13 | 0.03407465 |
| GO: 0002250 | adaptive immune response | BP | 297 | 109 | 50 | 2.89242E−13 | 0.998536556 |
| GO: 0071347 | cellular response to interleukin-1 | BP | 130 | 60 | 11 | 1.06612E−12 | 0.9999983 |
| GO: 1901700 | response to oxygen-containing compound | BP | 830 | 236 | 203 | 4.16441E−12 | 0.313783542 |
| GO: 0002521 | leukocyte differentiation | BP | 356 | 121 | 81 | 8.55489E−12 | 0.694659362 |
| GO: 0007249 | I-kappaB kinase/NF-kappaB signaling | BP | 205 | 80 | 44 | 1.96079E−11 | 0.811252848 |
| | GO Term: DOWNREGULATED | | | | | | |
| Column1 | LPS/cultured clot vs cultured clot | Ont | N | Up | Down | P. Up | P. Down |
| GO: 0031224 | intrinsic component of membrane | CC | 1865 | 405 | 570 | 0.001969453 | 4.87431E−15 |
| GO: 0071944 | cell periphery | CC | 2172 | 493 | 637 | 1.36951E−06 | 4.92262E−13 |
| GO: 0005886 | plasma membrane | CC | 2049 | 466 | 604 | 3.28892E−06 | 1.01892E−12 |
| GO: 0044255 | cellular lipid metabolic process | BP | 493 | 77 | 178 | 0.989143497 | 9.76827E−11 |
| GO: 0120025 | plasma membrane bounded cell projection | CC | 807 | 131 | 251 | 0.99141735 | 8.75343E−08 |
| GO: 0005887 | integral component of plasma membrane | CC | 494 | 139 | 163 | 3.31905E−07 | 5.22635E−07 |
| GO: 0034308 | primary alcohol metabolic process | BP | 32 | 0 | 21 | 1 | 7.64507E−07 |
| GO: 0046486 | glycerolipid metabolic process | BP | 232 | 37 | 84 | 0.923538416 | 1.06083E−05 |
| GO: 1901615 | organic hydroxy compound metabolic | BP | 228 | 39 | 83 | 0.837424219 | 1.13031E−05 |
| | process | | | | | | |
| GO: 0004888 | transmembrane signaling receptor activity | MF | 276 | 92 | 97 | 1.14262E−08 | 1.29311E−05 |
| GO: 0009395 | phospholipid catabolic process | BP | 22 | 4 | 15 | 0.649134878 | 1.45733E−05 |
| GO: 0006650 | glycerophospholipid metabolic process | BP | 201 | 30 | 74 | 0.957863239 | 1.82699E−05 |
| GO: 0006644 | phospholipid metabolic process | BP | 232 | 35 | 83 | 0.963227807 | 2.08884E−05 |
| GO: 0016042 | lipid catabolic process | BP | 135 | 23 | 54 | 0.79167843 | 2.11898E−05 |
| GO: 0071617 | lysophospholipid acyltransferase activity | MF | 16 | 0 | 12 | 1 | 2.38279E−05 |
| GO: 0016411 | acylglycerol O-acyltransferase activity | MF | 16 | 0 | 12 | 1 | 2.40985E−05 |
| GO: 0038023 | signaling receptor activity | MF | 374 | 114 | 123 | 5.77453E−08 | 3.74021E−05 |
| GO: 0060089 | molecular transducer activity | MF | 374 | 114 | 123 | 5.77453E−08 | 3.74021E−05 |
| GO: 0003779 | actin binding | MF | 204 | 26 | 73 | 0.994449835 | 4.36134E−05 |
| GO: 0006066 | alcohol metabolic process | BP | 168 | 28 | 63 | 0.846045154 | 4.96764E−05 |
| GO: 0098590 | plasma membrane region | CC | 409 | 75 | 131 | 0.716022802 | 5.83167E−05 |
| GO: 0008610 | lipid biosynthetic process | BP | 355 | 62 | 116 | 0.844777382 | 7.38181E−05 |
| GO: 0042572 | retinol metabolic process | BP | 13 | 0 | 10 | 1 | 0.000101286 |
| GO: 0044242 | cellular lipid catabolic process | BP | 100 | 14 | 41 | 0.940628158 | 0.000112021 |
| GO: 0034754 | cellular hormone metabolic process | BP | 36 | 4 | 19 | 0.940893065 | 0.000180965 |
| GO: 0043299 | leukocyte degranulation | BP | 446 | 99 | 138 | 0.06408957 | 0.000213609 |
| GO: 0016788 | hydrolase activity, acting on ester bonds | MF | 354 | 60 | 113 | 0.890888128 | 0.000221491 |
| GO: 0042171 | lysophosphatidic acid acyltransferase | MF | 14 | 0 | 10 | 1 | 0.000233947 |
| | activity | | | | | | |
| GO: 0016298 | lipase activity | MF | 48 | 9 | 23 | 0.609295625 | 0.000249238 |

TABLE 4
Linear regression (log-log) of fresh blood absolute leukocyte
counts as predictors of fresh blood OLR1
expression (TPM, RNAseq) (N = 11)

| | RSquare | RSquare adj | prob>\|t\| |
|---|---|---|---|
| Lymphocytes | 0.04 | −0.08 | 0.56 |
| Neutrophils | 0.43 | 0.36 | 0.04* |
| Monocytes | 0.00 | −0.12 | 0.96 |
| Eosinophils | 0.02 | −0.10 | 0.70 |
| Basophils | 0.28 | 0.19 | 0.12 |

*OLR1 Log10 = −3.24 + 4.60*neutrophil counts Log10
Table 4 is graphically plotted and depicted in FIG. 33.

In an embodiment, tests can therefore reveal the presence of AAT deficiency caused or not by factors that inactivate AAT or consume AAT activity. Cultured clot serum, but not fresh serum, contains PR3 and NE which deplete the ability of AAT to inhibit added NE enzyme as shown. As shown in FIG. 46 AAT is highly abundant in fresh serum forms an inhibitory complex with PR3 and NE. FS is normally devoid of PR3 and NE. AAT in healthy blood plasma is about 1000-fold excess over PR3 and NE detected in cultured clot serum. Purified NE (Sigma, Product #324681, 10 μg/mL final concentration) was completely inhibited by AAT present in 1:200 diluted fresh serum (FS) from Donors 1 and 2. In 1:2000 FS, a donor-dependent inhibition of NE was seen which may be partly due to different endogenous AAT levels. Cultured clot serum samples which contain PR3 and NE showed partial AAT deficiency towards the added NE enzyme. The % AAT inhibition test could be used to assess inherent AAT deficiency, or to screen drugs or treatments for their ability to increase or impair AAT activity. Methods: Donor 1 and Donor 2 provided blood samples to produce fresh serum (FS, 30 min RT) and cultured clot samples (4 h, 37° C.) without and with additives (100 ng/mL LPS, 10 mM beta-GP). Serum samples were diluted to 1:200 or 1:2000, then spiked or not with purified neutrophil elastase (NE, 0.02 U) to measure elastase activity at the optimal pH for proteinase 3 (pH 7.4) with a colorimetric substrate (N-methoxysuccinyl-Ala-Ala-Pro-Val p-nitroanilide (0.05 mg/mL MeOSUC-AAPV-pNA, Sigma, M4765). Proteinase 3 and elastase were previously detected in the cultured clot serum but not fresh serum of both donors. Donor 1 shows 100% inhibition of NE when combined with 1:200 or 1:2000 fresh serum, and reduced inhibitory activity of AAT when NE is combined with 1:200 or 1:2000 in cultured clot serum (top row, wells 3 & 4 vs fresh serum, well 1). Alpha-1 antitrypsin inhibition is higher than NE at 1:200 at the enzyme activity of NE tested. (4 μL NE was added to 1 mL TBS pH 7.4 prior to adding 50 μL to all sample wells containing 50 μL diluted serum or buffer-only, then 50 μL of substrate). Donor 2 shows elevated AAT inhibition in fresh serum (FIG. 46, well 1) and cultured clot serum (wells 2 to 4). These are enabling data for using cultured clot test to determine whether AAT deficiency is present.

FIG. 47 shows a kit. In one embodiment, the kit is portable. It contains miniaturized blood collection tube. Collection tube could be one or more, such as number of tubes are 2, 3, 4, 5, 10 or more. In an embodiment, a tube having blood of the user using the kit is which is heated in a small chamber with a battery-driven heating system with a display monitor and temperature sensor/timer. In another embodiment, heating system may use AC electric power. The chamber with heating system could be a device as described in an embodiment of the invention.

In an embodiment, tubes are color-coded. Color coded could be for differentiation for culturing the blood at room temperature (normal serum) and another for culture in the heating device. In an embodiment, tubes are made of clot-activating material as known to person skilled in the art. In an embodiment, caps are placed on either end of the glass capillary to avoid evaporation. Caps are made of biocompatible material as known to person skilled in the art. In an embodiment, tubes and caps are made of material that could be autoclaved. In an embodiment, tube are single use tubes. In some embodiments, tubes could be used multiple times. The tubes are incubated at desired temperature disclosed in one or more embodiments of the present invention.

After the incubation period, liquid expressed from the clot is applied to a lateral flow test with antibodies to the target and a positive control. In another embodiment, the device kit is a phlebotomy kit with 2 red vacutainer tubes. The portable heating device is manufactured so that one vacutainer tube can be inserted inside for 4 hours of incubation at 37 C. The device can optionally be programmed to cool to 4 degrees after 4 hours.

In an embodiment, the heating device may have an option to insert a test strip to read the liquid expressed from the clot, as described in U.S. Pat. No. 10,436,773B2 incorporated by reference in its entirety.

In an embodiment, the test strip is configured to detect sLOX-1 in the liquid and/or any biomolecule affecting the disease or providing a prognosis of the disease.

In an embodiment, present invention is further directed at a kit comprising: (a) a container containing a coagulation enhancing material in solution or in lyophilized form; (b) optionally, instructions for (i) use of the solution or (ii) device.

The kit may further comprise one or more of (iii) a buffer, (iv) a diluent, (v) a filter, (vi) a needle, or (v) a syringe. The container is preferably a bottle, a vial, a syringe or test tube; and it may be a multi-use container.

In an embodiment, kit may facilitate to carry a method to detect sLox-1 production at home or care center using blood of a patient or a user.

In an embodiment, the cultured clot system to monitor sLox-1 production before and after administration of chemotherapeutics or treatments that alter myeloid cell counts or myelopoesis (either intended or unintended), the cultured clot device could be used to demonstrate functional myelopoesis either on an individual patient basis or in clinical trials.

### Pharmaceutical Composition

In an embodiment, a precision medicine tool is developed that detects Lox-1 induction in an ex vivo thrombus and shedding of soluble Lox-1 (sLox-1) into the serum. The tool consists of a red vacutainer tube with as little as 0.5 mL and more ideally 3 or 4 mL of peripheral whole venous blood, that is placed for 4 hours at 37° C.

The data suggests that sLox-1 generated from a cultured clot may have cardioprotective activities. The cultured clot system may be used to create a sLox-1 based therapeutic that could prevent trauma-induced cardiomyopathy and diminish symptoms of acute coronary syndrome.

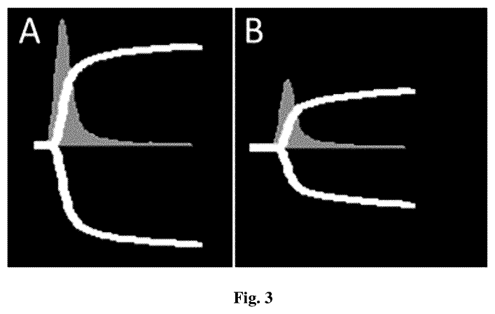

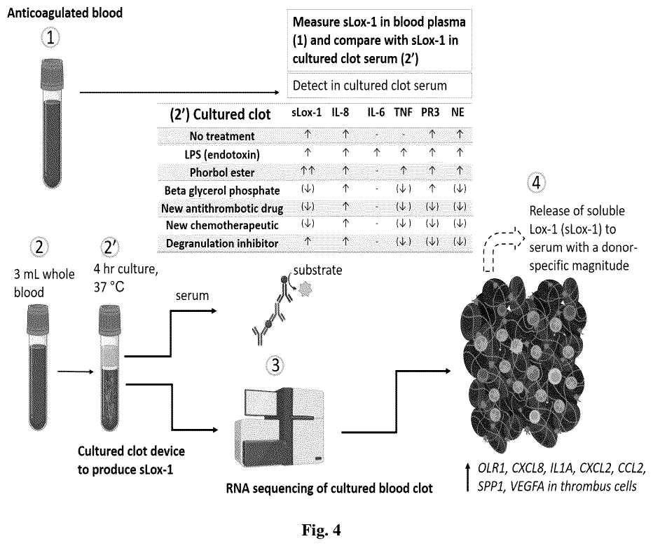

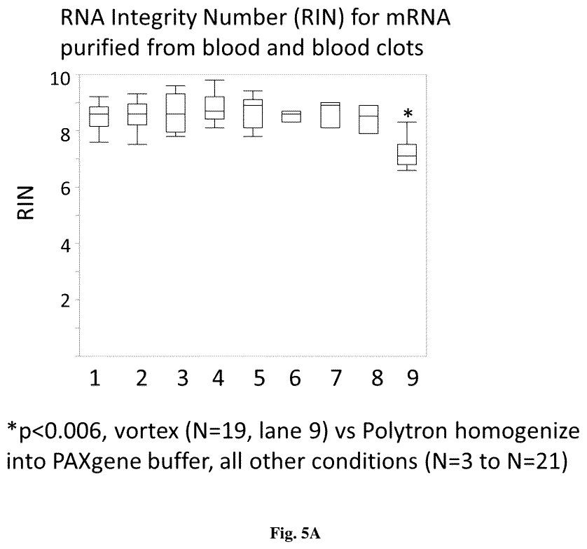

Data were generated that suggests cultured clot serum has an inhibitory effect on blood coagulation that may be due to accumulation of sLox-1 in the serum (FIG. 3). These data suggest that the cultured clot system may be used to generate sLox-1 for use a new biological anti-coagulant.

In FIG. 28 TER assay with HPAEC treated with 0.2 U/mL thrombin, 10% fresh serum, 10% cultured clot serum or 10% LPS/cultured clot serum produced by peripheral blood collected from 4 healthy donors (N=2 tests per condition for donors 1 and 2 and N=1 test per condition for combined samples from donors 3 and 4). Mean ±standard deviation (N=4 thrombin and N=5 for all other conditions). In the N=4 donors with variable WBC counts (min-max: 4.6 to 11.2 10∧6/mL), cultured clot serum showed min-max for sLox-1 (0.8 to 4.5 ng/mL) and IL-8 (2.0 to 7.1 ng/mL).

FIG. 28 shows data with N=5 measures show highly significant differences in cultured clot serum barrier-enhancing effects compared to control and fresh serum.

Interestingly, both cultured clot serum and LPS/cultured clot serum showed a similar and striking barrier-enhancing effect in the initial 4 hours of treatment that declined over 24 hours, with a mean normalized resistance (ohms) at 36 min. of 1.73±0.04 (cultured clot), 1.83±0.20 (LPS/cultured clot) vs. 1.01±0.11 (fresh serum, p<0.001, N=5 tests) and 0.48±0.03 (thrombin, p<0.001, N=4 tests, FIGS. 28 and 30)

In an embodiment, sLox-1 serves a protective role, then individuals with a more robust sLox-1 innate immune response may have a better prognosis for certain disease contexts. For example, individuals with elevated sLox-1 may better withstand lung injury due to oxygenation by mechanical ventilation.

Data in this study are important because they suggest a new mechanism for mature peripheral blood neutrophils to undergo rapid PMN-MDSC polarization and raise the possibility that sLox-1 is shed from neutrophils in pro-thrombotic disease states including COVID-19 and potentially ARDS.

In an embodiment, sLox-1 may serve as a novel cardioprotective agent or new biological anti-coagulant.

In an embodiment, sLox-1 is enriched by ConA sepharose bead affinity chromatography.

In an embodiment, sLox-1 may serve as a novel anti-thrombogenic agent. Anti-thrombogenic refers to a process of delaying clotting of blood or delaying activation of pro-platelets or platelets in peripheral blood.

In an embodiment, anti-thrombogenic agent is served in an anti-thrombogenic amounts. The term, “Anti-thrombogenic amounts”, without implying any limitation, “anti-thrombogenically effective” amount encompasses an amount that reduces some aspect of blood clotting to less than 100% of a maximal value, to less than 95% of a maximal value, to less than 90% of a maximal value, to less than 85% a maximal value, to less than 80%, to less than 75%, to less than 70%, to less than 65%, to less than 60%, to less than 55%, to less than 50%, to less than 40%, to less than 30%, to less than 20%, to less than 10%, to less than 5%, to less than 2%, to less than 1%, of the maximal value, and so on.

A “personalized pharmaceutical” shall mean specifically tailored therapies for one individual patient that will only be used for therapy in such individual patient, including actively personalized slox-1 using autologous patient's blood.

As used herein in its broadest meaning, the term “preventing” or “prevention” refers to preventing the disease or condition from occurring in a subject which has not yet been diagnosed as having it or which does not have any clinical symptoms.

As used herein, the term “treating” or “treatment”, as used herein, means reversing, alleviating, or inhibiting the progress of the disorder or condition to which such term applies, or one or more symptoms of such disorder or condition. A “therapeutically effective amount” is intended for a minimal amount of active agent which is necessary to impart therapeutic benefit to a subject. For example, a “therapeutically effective amount” to a patient is such an amount which induces, ameliorates, stabilises, slows down the progression or otherwise causes an improvement in the pathological symptoms, disease progression or physiological conditions associated with or resistance to succumbing to a disorder.

In an embodiment, sLox-1 could be used as a pharmaceutical composition.

As used herein, a “pharmaceutical composition” is a composition suitable for administration to a human being in a medical setting. Preferably, a pharmaceutical composition is sterile and produced according to GMP guidelines.

The pharmaceutical compositions comprise the protein extracted according to an embodiment of the invention, either in the free form or in the form of a pharmaceutically acceptable salt. As used herein, “a pharmaceutically acceptable salt” refers to a derivative of the protein extracted according to an embodiment of the invention, wherein the protein is modified by making acid or base salts of the agent. For example, acid salts are prepared from the free base (typically wherein the neutral form of the drug has a neutral —NH₂ group) involving reaction with a suitable acid. Suitable acids for preparing acid salts include both organic acids, e.g., acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methane sulfonic acid, ethane sulfonic acid, p-toluene sulfonic acid, salicylic acid, and the like, as well as inorganic acids, e.g., hydrochloric acid, hydrobromic acid, sulfuric acid, nitric acid phosphoric acid and the like. Conversely, preparation of basic salts of acid moieties which may be present on a peptide are prepared using a pharmaceutically acceptable base such as sodium hydroxide, potassium hydroxide, ammonium hydroxide, calcium hydroxide, trimethylamine or the like.

In an embodiment, composition is used for parenteral administration, such as subcutaneous, intradermal, intramuscular or oral administration. For this, the proteins extracted according to an embodiment or other molecules are dissolved or suspended in a pharmaceutically acceptable, preferably aqueous carrier. In addition, the composition can contain excipients, such as buffers, binding agents, blasting agents, diluents, flavors, lubricants, etc. The proteins can also be administered together with immune stimulating substances, such as cytokines. An extensive listing of excipients that can be used in such a composition can be, for example, taken from A. Kibbe, Handbook of Pharmaceutical Excipients (Kibbe, 2000). The composition can be used for a prevention, prophylaxis and/or therapy of coagulopathy and/or cardiopathy.

In an embodiment, the pharmaceutical composition is administered in a therapeutically effective amount.

As used herein, the term “therapeutically effective amount” refers to an amount that is sufficient or effective to prevent or treat (delay or prevent the onset of, prevent the progression of, inhibit, decrease or reverse) a disease or condition described or contemplated herein, including alleviating symptoms of such disease or condition. As used herein, the term “effective amount” or “therapeutically effective amount” of a compound is that amount of compound that is sufficient to provide a beneficial effect to the subject to which the compound is administered. Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Device

In an embodiment, device may be used to screen blood donors for sLox-1 levels in order to produce lots of blood plasma or serum with “high” and “low” sLox-1 levels, for specific clinical applications where the level of sLox-1 could affect the clinical outcome, for example, inhibition of coagulation in organ transplantation, pro-coagulant activity for survival from hemorrhage, or to discriminate between low platelets arising from inadequate platelet production or platelet activation in the circulation which according to this invention is expected to stimulate sLox-1 shedding into the blood plasma.

In an embodiment, the device may be as shown in FIG. 27.

In an embodiment, cultured clot device to screen agents that promote or inhibit one or more of the following: cell apoptosis, scramblase activity, flippase activity, ADAM17 activity, ADAM10 activity, alpha secretase activity, proteinase 3 activity, neutrophil elastase activity, neutrophil-platelet aggregation activity, FXIII activity, sLox-1 sheddase activity, tumor necrosis factor activation.

In an embodiment, the device has a means of heating and a tube. The tube could be Vacutainer tube. The heating could be an inbuilt heating technique. The heating method could be portable.

In an embodiment, the device is capable to maintain the temperature at around 37° C. The temperature could be maintained for less than 1 hour, 2 hours, 3 hours, 4 hours, 5 hours, 6 hours, 7 hours, 8 hours, 24 hours, 48 hours or more.

In an embodiment, the device may have a cooling mechanism to decrease the temperature at a desired cooling temperature such as but not limited around 4° C. In an embodiment, the device is capable to decrease the temperature at around 4° C. automatically or manually after a specified time of usage for example: 1 hour, 2 hours, 3 hours, 4 hours, 5 hours, 6 hours, 7 hours, 8 hours, 24 hours, 48 hours or more.

FIG. 24 shows that the cultured clot device could be used as a precision medicine device to test for drug-induced responses that aim to reduce or increase the abundance of cells expressing Lox-1 (i.e., PMN-MDSCs), or alter sLox-1, or mediators upstream of sLox-1 shedding, including ADAM17 sheddase activity, phosphatidylserine exposure which is needed for ADAM17 activation, apoptosis which causes phosphatidylserine exposure, or TNF activity which depends on ADAM17.

In an embodiment, fresh serum that was cultured for 30 minutes at room temperature failed to show the same highly increased barrier-enhancing function as clots cultured 4 hours at 37° C., suggesting the effect seen with cultured clot serum is due to accumulation of barrier-enhancing factors released from WBC such as PMN-MSCSs, encapsulated in the clot.

The optimized cultured clot system involves placing a red cap vacutainer tube with anionic clot-activator at 37° C. for 4 hours after which serum may be collected. Serum contains a mixture of barrier-reducing and barrier-enhancing factors. Cultured clot serum showed overall highly potent barrier-enhancing effects compared to purified factors analyzed in other studies.

In an embodiment, as shown in FIG. 27, test article is a drug, biomaterial, pathogen, endotoxin, phorbol ester, phosphatase inhibitor or activator, kinase inhibitor or activator, antibody, nanoparticles, cells, mRNA, lipid-mRNA nanoparticles, siRNA, CRISPR, cells, transfusion products, intended to modulate PMN-MDSC viability or activity, or oxidative stress, or ADAM17 activity or TNF activation or proteinase 3 activity or elastase activity or angiogenic activity.

An action we are taking adding the pro-coagulant is already a product—the red cap vacutainer tube. But nobody has said to put it at 37° C. as a device to produce sLox-1 or generate PMN-MDSCs. In an embodiment, temperature range is approximately 25° C. to 42° C.

In an embodiment, collecting shed sLox-1 from the cultured clot serum.

In an embodiment, fresh clot serum incubated for 45 min at room temperature has no sLox-1 as shown in FIG. 13B (condition FC, no PMA).

In an embodiment, device could detect other soluble factors (i.e., high IL8 to document cell viability, no IL6 to document freedom from infection, TNF, IL1A, IL8, VEGF, resolvins, oxylipids, etc or other factors upregulated in cultured clots) for concentration and/or bioactivity.

In an embodiment, the device is as described in US20220133192A1.

### Example 1

Bulk RNAseq of cultured whole blood clots (4 h, 37° C.) revealed that coagulation and endotoxin (LPS) elicit distinct transcriptional shifts compared to fresh plasma and cultured heparin blood (FIG. 42A) [4] and distinct metabolic shifts (FIG. 32A-C).

Coagulation was sufficient to induce cultured clot neutrophils to express PMN-MDSC signature genes including Lox-1 (FIG. 42A, panel B), and shed copious levels of sLox-1 to serum (FIG. 42C).

These responses were seen in 26 different male and female healthy donors with diverse age, ethnicity, and blood types. Contrary to current thinking [83, 84], we ruled out platelets as a source of sLox-1 [83] in an antibody validation study [57].

High-resolution mass spectrometry (MS) showed that cultured clot serum from 2 healthy donors contains proteinase 3 (PR3), a neutrophil enzyme, and activated FXIII (by depletion) (FIG. 42D). Fresh serum contained activated FXIII but no PR3 (FIG. 42D). Cultured clot serum showed potent endothelial barrier enhancing effects compared to fresh serum (FIG. 42E).

These data provide a clue that PMN-MDSCs may release soluble mediators that protect endothelial barrier function, including sLox-1. We observed high variation in sLox-1 release between donors (FIG. 42C). Interestingly, 7 donors who donated blood on several occasions showed a consistent amplitude of sLox-1 release that could reflect a personalized innate immune capacity for PMN-MDSC polarization. Platelet-derived oxidized lipids (4-HNE, from our MS lipidomics) could be a driver.

From these data and those of Korkmaz et al. [70], we reasoned that if sLox-1 has pulmonary protective activities, we should see an increased expression of Lox-1 in ARDS peripheral blood. We therefore studied ARDS whole blood microarray metadata collected the first 5 days of ICU [46, 85].

OLR1 (Lox-1) and PRTN3 (PR3) escalated in 3 patients who survived, along with oscillating levels of platelet marker CXCL7 (FIG. 43A, panels A-C) but not in 3 patients who died (FIG. 43A, panels D-F). Next, we analyzed study data from the ARDS-Omega trial (N=271) and found 3 sub phenotypes: patients with acute coagulopathy, low platelet oscillation and high mortality (lower left box, FIG. 43G), or low coagulopathy, low or high platelet oscillation and survival (upper 2 boxes, FIG. 43G). Low average platelet counts over entire hospital stay was a strong predictor of mortality (FIG. 43H). A pilot study of sLox-1 in ARDS Omega plasma from these 3 groups revealed an association of sLox-1 and 60-day survival (FIG. 43G & 3I, N=48). Coag28 (range: 0 to 28) is defined as platelet levels falling below 80,000 per μL between day 0 and 28 post-admission to ICU. The S.D. platelet counts is defined as the standard deviation of all platelet measures carried out between day 0 to day 60 post-admission to ICU.

Multivariate analysis of survival as a function of sLox-1, Omega oil treatment, ventilator-free days (VFD) and ICU-free days (ICUFD) showed an association of survival with sLox-1 (OR=25.3, p=0.056) but not treatment (OR=0.9, p=0.96), VFD (OR=0.68, p=0.68) or ICUFD (OR=3.1, p=0.42). These unique findings support the following hypothesis: Plasma sLox-1 is positively associated with 60-day survival in ARDS patients and activated platelets are sufficient to stimulate blood neutrophils to produce sLox-1.

### Example 2

Perform a multivariate analysis of acute plasma sLox-1 and 60-day survival in ARDS (Omega N=46, SAILS N=57, day 0, 3, 6) as the primary outcome. Time-dependent changes in sLox-1, other inflammatory biomarkers (IL-8, PR3, oxidized lipids), effect of treatment (Omega-3, statin), and sLox-1 concentration in plasma vs BAL (ALTA, N=20), will be explored as secondary outcomes. Our pilot data suggests that plasma sLox-1 will escalate in a subset of patients (FIG. 43A, panels A-F, FIG. 43B, panel C) and that these patients will have higher 60 day survival than patients with low sLox-1 or IL-8 levels that exceed sLox-1 levels. Plasma samples will be analyzed at day 0, 3, and 6 from the same subjects from the Omega & SAILS ARDS net trials (Table 2). SAILS specimens will allow us to study the potential effects of rosuvastatin, a drug that inhibited Lox-1 expression in endothelial cells in other studies [86].

Upon receipt of BioLINCC specimens, we will take inventory and stored them at −80° C. Under Biosafety Level 2 (BSL2) precautions, 20 samples at a time are thawed to remove a 50 μL aliquot for sLox-1 and IL-8 ELISA (BioTechne, 31.25 to 2000 pg/mL) and to prepare 3 frozen aliquots: 1 for PR3 ELISA, 1 for lipidomics, and 1 reserve aliquot. For Mass Spectrometry of lipids, 50 μL of plasma is extracted with chloroform/methanol/water (2:2:1) and analyzed by LC-MS with a Sciex QTRAP 4500 instrument with an ion spray voltage at 4500V. Flow rates and solvent mixtures were previously optimized to detect 4-HNE, lysophosphatidylcholine [87-89] (positive mode) and arachidonic acid, thromboxane B2, and EPA (negative mode) [90]. To collect information as to the potential tissue origin(s) of sLox-1 (i.e. lung vs.peripheral blood), sLox-1 levels are compared in BAL and plasma from the same patient in 18 donors from the ALTA trial.

We requested a minimal number of precious BAL specimens for this pilot sub-study. Sex, age across lifespan and treatment are balanced for all groups. A DASH healthy donor group will be used to test the effect of ARDS and rosuvastatin treatment on PR3 levels.

Statistical analysis: For our primary outcome, we will select a model by stepwise logistic regression considering all covariates to obtain the Odds Ratio (OR), standard error and p.values for sLox-1 in 103 OMEGA+SAILS (day 0, 3, or 6) or 169 ARDS specimens (power>0.9) that include ALTA (N=18) and already-collected pilot data N=48 (FIG. 39). We will additionally explore the role of day of sample collection (0, 3, 6) on sLox-1. In our pilot multivariate analysis of sLox-1 and 60-day survival, there was some evidence of association with ventilator-free days, ICU-free days, cardiovascular failure-free days, organ failure-free days, and coagulation failure-free days. We will examine possible sex-related differences and the effect of treatment (omega-3, statin, albuterol) and ARDS vs. healthy controls (Table 2). Secondary outcomes will analyze IL-8, PR3 and oxidized lipids vs. day-60 death, collection day, and sample type (BAL, plasma, ALTA samples). Pearson's correlation will be used to evaluate biomarker associations.

Expected results: sLox-1 will be predictive of survival. IL-8 (hyper-inflammatory) will be associated with mortality. sLox-1, PR3, and selected oxidized lipids will escalate with time. PR3 will be higher in ARDS vs. healthy control plasma (DASH). Treatment will have no effect on sLox-1.

### Example 3

Identify mechanisms of sLox-1 production by neutrophils using engineered cultured blood clots (healthy donors), pharmacological modulation of thromboinflammatory pathways and putative sheddases, and MS to profile coagulation factor activation and neutrophil mediators.

Current data suggest that activated platelets are the major mediators driving neutrophils to produce sLox-1. Thrombin, for example, had no effect on sLox-1 production in our HEK293 human Lox-1 overexpression system [54]. The enzymes responsible for sLox-1 shedding are under intense debate [63] and not yet identified in neutrophils. Close scrutiny of PR3 leads hypothesize it is a novel neutrophil-specific sheddase of sLox-1. PR3 is harbored in azurophilic and secretory granules of neutrophils [64]. Upon release, PR3 associates with the neutrophil cell surface via CD177 in cholesterol-rich lipid rafts where Lox-1 is located [64, 65]. PR3 activity is believed to preserve endothelial barrier function during neutrophil extravasation [66-68]. Previous studies showed reduced sLox-1 shedding by cells exposed to phenylmethylsulfonyl fluoride (PMSF), a broadband serine protease inhibitor [48] or TAPI-1 (for ADAM17, TNF convertase) [69] but failed to address potential toxicity to explain these results. In addition, close scrutiny of published sLox-1 sheddase sites from MS studies [91, 92] revealed that putative human and bovine sLox-1 cleavage sites have little shared homology except for a common trypsin digestion motif (R86, K89, R88). One study used trypsin digestion during sample preparation [70]. The proposed sLox-1 cleavage motif is missing an aliphatic P1′ residue used by ADAM10 and ADAM17 to recognize a substrate [71]. By contrast, the human Lox-1 cleavage region (QISA87R88QQA) [92] has a very striking match with the PR3 cleavage site in IL-8 (AVLPRSA↓RKEL) [93, 94]. The critical alanine residue in a potential sLox-1 PR3 cleavage site is conserved across species, but the R88 residue and surrounding amino acids are not conserved across species and do not share many homologies with ADAM10 or ADAM17 cleavage motifs [95] (Table 5). The published cleavage sites for sLox-1 in bovine and human produce a non-conserved sLox-1 N-terminus, with NH2-Glutamine for human and NH2-Serine for bovine (Table 5). These collective observations suggest that PR3 could be a sheddase that releases sLox-1 from clot-activated neutrophils as a feedback mechanism to protect vascular barrier function. Potentially harmful bystander elastase activity of PR3 [73] could be suppressed by high levels of alpha-1 antitrypsin or ceruloplasmin that we detected by LC-MS in plasma and serum [74].

TABLE 5
Published sLox-1 N-termini (human, bovine) and
candidate sheddase cleavage motifs

| | Lox-1 amino acid sequence | |
|---|---|---|
| | around the published | Putative PR3 cleavage |
| | human and bovine | site in Lox-1 |
| Species | sLox-1 cleavage site | to generate sLox-1 |
| human | EGQISAR↓QQAEEASQ | EGAISA↓RQQAEEASQ |
| | (Biocca et al. 2013) | |
| bovine | EGQILAQRR↓SEK↓SAQ | EGQILA↓QRRSEKSAQ |
| | (Murase et al. 2000) | |
| porcine | EGQALAQRQAEKSSQ | EGQALA↓QRQAEKSSQ |
| | (R followed by Q) | |
| rabbit | EGQVLAQQQAEAASQ | EGQVLA↓QQQAEAASQ |
| | (missing R and K residues) | |
| rat | EGQMSAQKKAENASQ | EGQMSA↓QKKAENASQ |
| | (K followed by A) | |
| mouse | EGQMLAQQKAENTSQ | EGQMLA↓QQKAENTSQ |
| | (K followed by A) | |
| | ADAM17 and ADAM10 | PR3 cleavage motif |
| | cleavage motifs | |
| | (Caescu et al. 2010) | (Korkmaz et al. 2010; |
| | | Crisford et al. 2018) |
| ADAM17 | PLAQA↓VRSS (TNF) | |
| | PVAAA↓VVS (TGF-alpha) | |
| | QETNR↓SFS (L-selectin) | |
| ADAM17 and | SLPVQ↓DSS (IL-6R) | |
| ADAM10 | GLTLP↓VEN (HB-EGF) | |
| ADAM10 | WWELR↓HAG (EGF) | |
| | VDLFY↓LLRG (BTC) | |
| | SSLEK↓QIG (FasL) | |
| PR3 | | AVLPRSA↓KEL (IL-8-human) |
| | | AVVA↓SELR (MIP-2-murine) |
| | | P4-P3-P2-P1↓P1-P2-P3 where P4, |
| | | P3 and P1 are hydrophobic |
| | | and P1 Val-Xaa or Ala-Xaa. |

The goal of this experiment is to identify pro-coagulant conditions that are necessary and sufficient to induce neutrophils to produce sLox-1. Fully informed consenting volunteers (healthy, diverse ethnicity, 18-89 years old, sex as a biological variable with 50% male, 50% female) are enrolled for blood collection by a certified phlebotomist. Blood samples are distributed in small aliquots (0.5 mL) in sterile glass culture tubes with vented steel caps under a biosafety cabinet, combined with mediators, and placed at 37° C. for 4 h [73]. We can easily screen 20 conditions with 20 mL of peripheral blood per donor, including 1 EDTA tube for manual white blood cell counts, smears for differential counts, plasma and 1 red cap tube for fresh serum controls. In brief, we will identify optimal conditions that promote or block neutrophil-platelet aggregate (NPA) formation in cultured clots (N=2M, N=2 F per screening assay). To inhibit NPA, whole blood prior to clot culture will be treated with (1) functional blocking L-selectin antibody (suppresses neutrophil-platelet binding) and prostacyclin (PGI2, blocks platelet aggregation) [96], (2) P2Y12 antagonist [97], (3) functional blocking anti-CD15 (PADGEM) antibody [62] or (4) 10 mM beta-glycerol phosphate to inhibit ATP to ADP conversion [73]. Treatments to promote NPA include arachidonic acid, ADP (P2Y12 agonist), ristocetin (promotes platelet agglutination), autologous platelets, thrombosomes (lyophilized platelets with 85% phosphatidylserine surfaces) [98] and phorbol ester [99].

For sLox-1 sheddase inhibition studies, cultured clots will be spiked with PR3 inhibitor elafin, or PMSF, and use SLPI (elastase inhibitor) as a negative control [100, 101]. Positive controls include unmodified cultured clots. Negative controls include cultured clots generated with heparin anticoagulated blood depleted of (1) neutrophils with (CD15-magnetic beads) [37] or (2) platelets (differential centrifugation), followed by reversal of anticoagulation (heparinase [102] or chitosan [103, 104]). Other negative controls include cultured heparin blood, fresh clot serum and plasma for baseline sLox-1 [73]. After 4 h of culture at 37° C., serum is cleared (1300 xg, 10 min) and analyzed by ELISA (sLox-1, IL-8, PR3), MS proteomics (PR3, FXIII) and lipidomics (AA, LPC, DHA, EPA, TBX2, HETE, 4-HNE, LTB4).

We will then identify the optimal conditions and analyze 1 NPA agonist, 1 NPA antagonist, and 1 sheddase inhibitor condition vs. controls in blood samples from 16 healthy donors (8 male, 8 female). NPA formation will be measured by flow cytometry (double immunohistofluorescence, CD41b & CD15 or Lox-1) of cells released by mechanical disruption of clots [37, 105]. A final study with purified neutrophils (N=4 donors) will allow us to confirm whether sLox-1 release can be controlled by individual cellular components and drugs without coagulum present. MS lipidomics is carried out as described [87-89]. MS untargeted proteomics of serum is used to confirm FXIII activation and PR3 and/or neutrophil elastase release as shown (FIG. 44) and to discover other mediators. Samples will be depleted of the major 14 abundant serum proteins (high select spin columns) and treated with rebuff red with ammonium carbonate. BCA is used to measure protein concentration. 20 micrograms protein will be reduced using DTT, alkylated with IAA, and digested with trypsin or LysC enzyme overnight. Samples are desalted and 1 microgram is injected into an Orbitrap Exploris 480 instrument [61, 84]. Samples will be acquired in a data independent mode and data files will be searched against human proteome database for protein identification and quantification.

In other experiments, sLox-1 purified by ConA sepharose from cultured clot serum will be fragmented with CNBr or LysC to avoid cleavage within the putative PR3 domain prior to MS analyses. Our group and other successfully used this approach to purify sLox-1 from conditioned medium of Lox-1-HEK293 cells [57, 106]. Statistical analyses: One sided Student's t-test (N=4) and analysis of the variance of the mean with post-hoc correction for multiple analyses (N=16) will be used to compare the effect of test condition on sLox-1, PR3, lipid mediators and factors identified in MS discovery proteomics. IL-8 will be used to monitor cell viability. A matched pair design will adjust for donor-specific sLox-1 production. Results from this experiment will be useful for interpreting clinical sample results.

Expected results: sLox-1 will not be released from cultured clots devoid of neutrophils or platelets. Conditions can be identified that suppress or enhance neutrophil-platelet aggregate (NPA) formation in whole blood by at least 1.5 fold. Cultured clots with suppressed or enhanced NPA will produce at least 1 standard deviation lower or higher, respectively, sLox-1 than untreated cultured clots. sLox-1 shedding will be inhibited by elafin PR3 inhibitor but not neutrophil elastase inhibitor SLPI. For MS analyses, all cultured clot serum will show higher serum AA, TBX2, activated FXIII, and PR3 than cultured clots including samples treated by elafin. NPA formation may stimulate resolvin production (i.e., arachidonate conversion to 5-HETE, 11-HETE instead of thromboxane A2 or eicosanoids) [105].

### Example 4

The cultured clot device will be used to as a test to measure or monitor functional myelopoesis and functional innate immune responses in a peripheral blood sample. Myelopoesis occurs in the bone marrow where myeoloid progenitors give rise to cells that comprise the innate immune repertoire: granulocytes (neutrophils, basophils, eosinophils), monocytes, and megakaryocytes/platelets. Disease states and some therapies cause transient or prolonged thrombocytopenia and/or neutrophil depletion. Some therapies (such as Neupogen, G-CSF) are used to restore neutrophil levels for example after chemotherapy. The cultured clot device will be used to document peripheral blood innate immune function and to monitor the loss and/or restoration of innate immune function after administering a treatment. This monitoring can be used as a proxy for documenting healthy myelopoesis in the bone marrow. In the context of cancer, if myelopoesis in the bone marrow is unaffected by chemotherapy or radiation therapy then functional innate immune responses can be restored within a month or two following intervention.

The patient blood sample is taken prior to treatment and sLox-1 levels in cultured clot serum (and potentially other factors: IL-8, IL-6, PR3, elastin) are measured. Then another blood sample is taken after treatment (one time or more than one time if monitoring is desired). If the treatment induces thrombocytopenia, non-responsive platelets, or neutrophil depletion this is expected to cause a reduction in sLox-1 levels. Restoration of functional platelet and neutrophil counts will restore the sLox-1 levels to baseline. The sLox-1 cultured clot test could therefore be used to monitor restoration of a functional and mature innate immune system. This monitoring could be useful for identifying the timing for course of multiple anti-cancer treatments that require restoration of innate immunity before treatment.

In another context, patient has innate immune deficiency or innate immune paralysis. The sLox-1 cultured clot test can be administered before and after therapeutic treatment to determine whether sLox-1 levels can be increased following administration of a drug, nutrient, gene therapy, or other intervention intended to restore a functional innate immune responses. If a treatment may induce neutrophIL1A and excessive platelet levels, sLox-1 levels are expected to rise in cultured clot serum after the intervention. Monitoring patient cultured clot sLox-1 levels could demonstrate restoration of homeostasis in bone marrow myelopoesis.

### Example 5

The cultured clot device will be manufactured as a light-weight portable testing kit to permit the user to determine innate immune function and relative risks of contracting a contagious disease, either at home or in remote locations where sophisticated blood cell differential counting instruments are not available. A sterile finger stick blood sample is collected in a glass tube and inserted in the heating device. The liquid expressed after 4 hours (for example) is submitted to an sLox-1 detection test with in-built positive and negative controls. The detection system could be an electronic biosensor, solid-phase immunodetection test, or other biosensing device. Development of a sLox-1 signal will be used to demonstrate functional innate immunity and presence of functional platelet and neutrophil levels.

### Incorporation by References

All references, including granted patents and patent application publications, referred herein are incorporated herein by reference in their entirety.

### Patent/Patent Application Publication

1. US20220133192A1
2. US20040120942A1
3. U.S. Pat. No. 4,359,463A

### RESEARCH ARTICLES

1. Agrawal A, Zhuo H, Brady S, Levitt J, Steingrub J, Siegel MD, et al. Pathogenetic and predictive value of biomarkers in patients with ALI and lower severity of illness: results from two clinical trials. Am J Physiol Lung Cell Mol Physiol. 2012;303: L634-639. doi:10.1152/ajplung.00195.2012
2. Matthay MA, Zemans RL, Zimmerman GA, Arabi YM, Beitler JR, Mercat A, et al. Acute respiratory distress syndrome. Nat Rev Dis Primers. 2019;5: 18. doi:10.1038/s41572-019-0069-0
3. Amgalan A, Othman M. Hemostatic laboratory derangements in COVID-19 with a focus on platelet count. Platelets. 2020; 1-6. doi:10.1080/09537104.2020.1768523
4. Verhoef PA, Kannan S, Sturgill JL, Tucker EW, Morris PE, Miller AC, et al. Severe Acute Respiratory Syndrome-Associated Coronavirus 2 Infection and Organ Dysfunction in the ICU: Opportunities for Translational Research. Critical Care Explorations. 2021;3: e0374. doi:10.1097/CCE.0000000000000374
5. Setua S, Thangaraju K, Dzieciatkowska M, Wilkerson RB, Nemkov T, Lamb DR, et al. Coagulation potential and the integrated omics of extracellular vesicles from COVID-19 positive patient plasma. Sci Rep. 2022;12: 22191. doi:10.1038/s41598-022-26473-8
6. Zhou H, Li Z, Liang H, Yan Z. Thrombocytopenia and platelet count recovery in patients with sepsis-3: a retrospective observational study. Platelets. 2022;33: 612-620. doi:10.1080/09537104.2021.1970124
7. Wang T, Liu Z, Wang Z, Duan M, Li G, Wang S, et al. Thrombocytopenia Is Associated with Acute Respiratory Distress Syndrome Mortality: An International Study. PLoS One. 2014;9: e94124. doi:10.1371/journal.pone.0094124
8. Yao Y, Cao J, Wang Q, Shi Q, Liu K, Luo Z, et al. D-dimer as a biomarker for disease severity and mortality in COVID-19 patients: a case control study. Journal of Intensive Care. 2020;8: 49. doi:10.1186/s40560-020-00466-z
9. Zhang L, Yan X, Fan Q, Liu H, Liu X, Liu Z, et al. D-dimer levels on admission to predict in-hospital mortality in patients with Covid-19. Journal of Thrombosis and Haemostasis. 2020;18: 1324-1329. doi:10.1111/jth.14859
10. Kangelaris KN, Prakash A, Liu KD, Aouizerat B, Woodruff PG, Erle DJ, et al. Increased expression of neutrophil-related genes in patients with early sepsis-induced ARDS. Am J Physiol Lung Cell Mol Physiol. 2015;308: L1102-L1113. doi:10.1152/ajplung.00380.2014
11. Perdomo J, Leung HHL, Ahmadi Z, Yan F, Chong JJH, Passam FH, et al. Neutrophil activation and NETosis are the major drivers of thrombosis in heparin-induced thrombocytopenia. Nat Commun. 2019;10: 1322. doi:10.1038/s41467-019-09160-7
12. Amparo Blanch-Ruiz M, Ortega-Luna R, Gomez-Garcia G, Angeles Martinez-Cuesta M, Alvarez A. Role of Neutrophil Extracellular Traps in COVID-19 Progression: An Insight for Effective Treatment. Biomedicines. 2022;10: 31. doi:10.3390/biomedicines10010031
13. Weber B, Lackner I, Gebhard F, Miclau T, Kalbitz M. Trauma, a Matter of the Heart-Molecular Mechanism of Post-Traumatic Cardiac Dysfunction. IJMS. 2021;22: 737. doi:10.3390/ijms22020737
14. Trappenburg MC, van Schilfgaarde M, Marchetti M, Spronk HM, Cate H t., Leyte A, et al. Elevated procoagulant microparticles expressing endothelial and platelet markers in essential thrombocythemia. Haematologica. 2009;94: 911-918. doi:10.3324/haemato1.13774
15. Bick RL. Disseminated Intravascular Coagulation: Pathophysiological Mechanisms and Manifestations. Semin Thromb Hemost. 1998;24: 3-18. doi:10.1055/s-2007-994971
16. Baker CJ, Smith SA, Morrissey JH. Polyphosphate in thrombosis, hemostasis, and inflammation. Res Pract Thromb Haemost. 2019;3: 18-25. doi:10.1002/rth2.12162
17. Nicolay JP, Thorn V, Daniel C, Amann K, Siraskar B, Lang F, et al. Cellular stress induces erythrocyte assembly on intravascular von Willebrand factor strings and promotes microangiopathy. Scientific Reports. 2018;8: 10945. doi:10.1038/s41598-018-28961-2
18. Hoemann CD, Rivard GE. Chitosan-Platelet Interactions. Chitosan for Biomaterials III. Springer International Publishing; 2021. pp. 319-342. Available: https://www.springerprofessional.de/en/chitosan-platelet-interactions/19706434
19. Panteleev MA, Ovanesov MV, Kireev DA, Shibeko AM, Sinauridze EI, Ananyeva NM, et al. Spatial Propagation and Localization of Blood Coagulation Are Regulated by Intrinsic and Protein C Pathways, Respectively. Biophysical Journal. 2006;90: 1489-1500. doi:10.1529/biophysj.105.069062
20. Maas C, Renné T. Coagulation factor XII in thrombosis and inflammation. Blood. 2018;131: 1903-1909. doi:10.1182/blood-2017-04-569111
21. Berckmans R, Nieuwland R, Boing A, Romijn F, Hack CE, Sturk A. Cell-derived Microparticles Circulate in Healthy Humans and Support Low Grade Thrombin Generation. Thromb Haemost. 2001;85: 639-649. doi:10.1055/s-0037-1615646
22. Contreras-García A, D′Elía NL, Desgagne M, Lafantaisie-Favreau C-H, Rivard G-E, Ruiz J-C, et al. Synthetic anionic surfaces can replace microparticles in stimulating burst coagulation of blood plasma. Colloids and Surfaces B: Biointerfaces. 2019;175: 596-605. doi:10.1016/j.colsurfb.2018.11.066
23. Sabbione F, Keitelman IA, Iula L, Ferrero M, Giordano MN, Baldi P, et al. Neutrophil Extracellular Traps Stimulate Proinflammatory Responses in Human Airway Epithelial Cells. J Innate Immun. 2017;9: 387-402. doi:10.1159/000460293
24. Kaplan MJ, Radic M. Neutrophil Extracellular Traps: Double-Edged Swords of Innate Immunity. JI. 2012;189: 2689-2695. doi:10.4049/jimmunol.1201719
25. Schmaier AH, Stavrou EX. Factor XII-What's important but not commonly thought about. Research and Practice in Thrombosis and Haemostasis. 2019;3: 599-606. doi:10.1002/rth2.12235
26. Kaplan AP, Joseph K, Silverberg M. Pathways for bradykinin formation and inflammatory disease. Journal of Allergy and Clinical Immunology. 2002;109: 195-209. doi:10.1067/mai.2002.121316
27. Butenas S, Mann KG. Blood coagulation. Biochemistry (Mosc). 2002;67: 3-12. doi:10.1023/a:1013985911759
28. Marchand C, Bachand J, PÃ\textcircled{C}rinÃ^{a}t J, Baraghis E, Lamarre M, Rivard GE, et al. C3, C5, and factor B bind to chitosan without complement activation. J Biomed Mater Res. 2010;93: 1429-41. doi:10.1002/jbm.a.32638
29. Sawamura T, Kume N, Aoyama T, Moriwaki H, Hoshikawa H, Aiba Y, et al. An endothelial receptor for oxidized low-density lipoprotein. Nature. 1997;386: 73-77. doi:10.1038/386073a0
30. Kume N, Kita T. Lectin-Like Oxidized Low-Density Lipoprotein Receptor-1 (LOX-1) in Atherogenesis. Trends in Cardiovascular Medicine. 2001;11: 22-25. doi:10.1016/S1050-1738(01)00079-2
31. Hofnagel O, Luechtenborg B, Stolle K, Lorkowski S, Eschert H, Plenz G, et al. Proinflammatory Cytokines Regulate LOX-1 Expression in Vascular Smooth Muscle Cells. Arteriosclerosis, Thrombosis, and Vascular Biology. 2004;24: 1789-1795. doi:10.1161/01.ATV.0000140061.89096.2b
32. Mehta JL, Chen J, Hermonat PL, Romeo F, Novelli G. Lectin-like, oxidized low-density lipoprotein receptor-1 (LOX-1): A critical player in the development of atherosclerosis and related disorders. Cardiovasc Res. 2006;69: 36-45. doi:10.1016/j.cardiores.2005.09.006
33. Pirillo A, Norata GD, Catapano AL. LOX-1, OxLDL, and Atherosclerosis. Mediators of Inflammation. 2013;2013: e152786; 1-12. doi:10.1155/2013/152786
34. Akhmedov A, Sawamura T, Chen C-H, Kraler S, Vdovenko D, Lüscher TF. Lectin-like oxidized low-density lipoprotein receptor-1 (LOX-1): a crucial driver of atherosclerotic cardiovascular disease. Eur Heart J. 2021;42: 1797-1807. doi:10.1093/eurheartj/ehaa770
35. Tatsuguchi M, Furutani M, Hinagata J, Tanaka T, Furutani Y, Imamura S, et al. Oxidized LDL receptor gene (OLR1) is associated with the risk of myocardial infarction. Biochemical and Biophysical Research Communications. 2003;303: 247-250. doi:10.1016/S0006-291X(03)00326-7
36. Chen M, Masaki T, Sawamura T. LOX-1, the receptor for oxidized low-density lipoprotein identified from endothelial cells: implications in endothelial dysfunction and atherosclerosis. Pharmacology & Therapeutics. 2002;95: 89-100. doi:10.1016/S0163-7258(02)00236-X
37. Condamine T, Dominguez GA, Youn J-I, Kossenkov AV, Mony S, Alicea-Torres K, et al. Lectin-type oxidized LDL receptor-1 distinguishes population of human polymorphonuclear myeloid-derived suppressor cells in cancer patients. Sci Immunol. 2016;1: aaf8943. doi:10.1126/s ciimmunol.aaf8943
38. Veglia F, Sanseviero E, Gabrilovich DI. Myeloid-derived suppressor cells in the era of increasing myeloid cell diversity. Nat Rev Immunol. 2021;21: 485-498. doi:10.1038/s41577-020-00490-y
39. Ryan S, Taylor CT, McNicholas WT. Selective activation of inflammatory pathways by intermittent hypoxia in obstructive sleep apnea syndrome. Circulation. 2005;112: 2660-2667. doi:10.1161/CIRCULATIONAHA.105.556746
40. Kent BD, Ryan S, McNicholas WT. Obstructive sleep apnea and inflammation: Relationship to cardiovascular co-morbidity. Respiratory Physiology & Neurobiology. 2011;178: 475-481. doi:10.1016/j.resp.2011.03.015
41. Hunyor I, Cook KM. Models of intermittent hypoxia and obstructive sleep apnea: molecular pathways and their contribution to cancer. American Journal of Physiology-Regulatory, Integrative and Comparative Physiology. 2018;315: R669—R687. doi:10.1152/ajpregu.00036.2018
42. Leonard J. Creating an in vitro model for acute inflammation using tissue-engineered blood clots. Masters thesis, George Mason University. 2021.
43. Aguillón JC, Escobar A, Ferreira V, Aguirre A, Molina MC, Ferreira A. Daily production of human tumor necrosis factor in lipopolysaccharide (LPS)-stimulated ex vivo blood culture assays. European Cytokine Network. 2001;12: 105-110. doi:PMID: 11282553
44. Leonard J, Lawson M, Gillevet P, Espina V, Hoemann CD. Hypoxic whole blood clots induce an immune response: A potential model for ARS and Covid-19. San Diego; 2020.
45. Parnell GP, Tang BM, Nalos M, Armstrong NJ, Huang SJ, Booth DR, et al. Identifying Key Regulatory Genes in the Whole Blood of Septic Patients to Monitor Underlying Immune Dysfunctions: Shock. 2013;40: 166-174. doi:10.1097/SHK.0b013e31829ee604
46. Parnell GP. GEO DataSet: Severe influenza A infection: whole blood. [cited 11 Oct 2023]. Available: https://www.ncbi.nlm.nih.gov/sites/GDSbrowser?acc=GDS3919
47. Sommer A, Kordowski F, Bach J, Maretzky T, Evers A, Andrä J, et al. Phosphatidylserine exposure is required for ADAM17 sheddase function. Nat Commun. 2016;7: 11523. doi:10.1038/ncomms11523
48. Sakuragi T, Kosako H, Nagata S. Phosphorylation-mediated activation of mouse Xkr8 scramblase for phosphatidylserine exposure. Proceedings of the National Academy of Sciences. 2019;116: 2907-2912. doi:10.1073/pnas.1820499116
49. Kamata H, Honda S, Maeda S, Chang L, Hirata H, Karin M. Reactive Oxygen Species Promote TNFα-Induced Death and Sustained JNK Activation by Inhibiting MAP Kinase Phosphatases. Cell. 2005;120: 649-661. doi:10.1016/j.cell.2004.12.041
50. Combadière B, Adam L, Guillou N, Quentric P, Rosenbaum P, Dorgham K, et al. LOX-1-Expressing Immature Neutrophils Identify Critically-Ill COVID-19 Patients at Risk of Thrombotic Complications. Front Immunol. 2021;12: 752612. doi:10.3389/fimmu.2021.752612
51. Hayashida K. Serum Soluble Lectin-Like Oxidized Low-Density Lipoprotein Receptor-1 Levels Are Elevated in Acute Coronary Syndrome. Circulation. 2005;112: 812-818. doi:10.1161/CIRCULATIONAHA.104.468397
52. Moriwaki H, Kume N, Kataoka H, Murase T, Nishi E, Sawamura T, et al. Expression of lectin-like oxidized low density lipoprotein receptor-1 in human and murine macrophages: upregulated expression by TNF-α. FEBS Letters. 1998;440: 29-32. doi:10.1016/S0014-5793(98)01414-8
53. Kakutani M, Masaki T, Sawamura T. A platelet-endothelium interaction mediated by lectin-like oxidized low-density lipoprotein receptor-1. PNAS. 2000;97: 360-364. doi:10.1073/pnas.97.1.360
54. Li D, Mehta JL. Antisense to LOX-1 Inhibits Oxidized LDL-Mediated Upregulation of Monocyte Chemoattractant Protein-1 and Monocyte Adhesion to Human Coronary Artery Endothelial Cells. Circulation. 2000;101: 2889-2895. doi:10.1161/01.CIR.101.25.2889
55. Mentrup T, Cabrera-Cabrera F, Schroder B. Proteolytic Regulation of the Lectin-Like Oxidized Lipoprotein Receptor LOX-1. Front Cardiovasc Med. 2021;7: 594441. doi:10.3389/fcvm.2020.594441
56. Haywood-Watson RJ, Holcomb JB, Gonzalez EA, Peng Z, Pati S, Park PW, et al. Modulation of Syndecan-1 Shedding after Hemorrhagic Shock and Resuscitation. McNeil P, editor. PLoS ONE. 2011;6: e23530. doi:10.1371/journal.pone.0023530
57. Leonard J, Hu C-H, Veneziano R, Hoemann CD. Investigation of Lox-1 Primary Structure with 6 Anti-Lox-1 Antibodies Suggests that Human Platelets Do Not Express the 37 kDa Lox-1, 24 kDa Soluble Lox-1 or OLR1, the mRNA Encoding Lox-1. Rochester, NY; 2023. Available: https://papers.ssrn.com/abstract=4544447
58. Nakayama F, Nishihara S, Iwasaki H, Kudo T, Okubo R, Kaneko M, et al. CD15 Expression in Mature Granulocytes Is Determined by α1,3-Fucosyltransferase IX, but in Promyelocytes and Monocytes by α1,3-Fucosyltransferase IV *. Journal of Biological Chemistry. 2001;276: 16100-16106. doi:10.1074/jbc.M007272200
59. Lambert C, Preijers FWMB, Yanikkaya Demirel G, Sack U. Monocytes and macrophages in flow: an ESCCA initiative on advanced analyses of monocyte lineage using flow cytometry. Cytometry B Clin Cytom. 2017;92: 180-188. doi:10.1002/cyto.b.21280
60. Bronte V, Brandau S, Chen S-H, Colombo MP, Frey AB, Greten TF, et al. Recommendations for myeloid-derived suppressor cell nomenclature and characterization standards. Nat Commun. 2016;7: 12150. doi:10.1038/ncomms12150
61. Kaiser R, Escaig R, Erber J, Nicolai L. Neutrophil-Platelet Interactions as Novel Treatment Targets in Cardiovascular Disease. Frontiers in Cardiovascular Medicine. 2022;8. Available: https://www.frontiersin.org/articles/10.3389/fcvm.2021.824112
62. Larsen E, Palabrica T, Sajer S, Gilbert GE, Wagner DD, Furie BC, et al. PADGEM-dependent adhesion of platelets to monocytes and neutrophils is mediated by a lineage-specific carbohydrate, LNF III (CD15). Cell. 1990;63: 467-474. doi:10.1016/0092-8674(90)90443-i
63. Ito T, Thachil J, Asakura H, Levy JH, Iba T. Thrombomodulin in disseminated intravascular coagulation and other critical conditions-a multi-faceted anticoagulant protein with therapeutic potential. Crit Care. 2019;23: 280. doi:10.1186/s13054-019-2552-0
64. Panigada M, Cucino A, Spinelli E, Occhipinti G, Panarello G, Novembrino C, et al. A Randomized Controlled Trial of Antithrombin Supplementation During Extracorporeal Membrane Oxygenation. Critical Care Medicine. 2020;48: 1636. doi:10.1097/CCM.0000000000004590
65. Gabrilovich DI, Nagaraj S. Myeloid-derived suppressor cells as regulators of the immune system. Nat Rev Immunol. 2009;9: 162-174. doi:10.1038/nri2506
66. Schulte-Schrepping J, Reusch N, Paclik D, Baßler K, Schlickeiser S, Zhang B, et al. Severe COVID-19 Is Marked by a Dysregulated Myeloid Cell Compartment. Cell. 2020;182: 1419-1440.e23. doi:10.1016/j.cell.2020.08.001
67. Coudereau R, Waeckel L, Cour M, Rimmele T, Pescarmona R, Fabri A, et al. Emergence of immunosuppressive LOX-1+ PMN-MDSC in septic shock and severe COVID-19 patients with acute respiratory distress syndrome. Journal of Leukocyte Biology. 2022;111: 489-496. doi:10.1002/JLB.4COVBCR0321-129R
68. Kume N, Moriwaki H, Kataoka H, Minami M, Murase T, Sawamura T, et al. Inducible Expression of LOX-1, a Novel Receptor for Oxidized LDL, in Macrophages and Vascular Smooth Muscle Cells. Annals of the New York Academy of Sciences. 2000;902: 323-327. doi:10.1111/j.1749-6632.2000.tb06332.x
69. Kume N, Murase T, Moriwaki H, Aoyama T, Sawamura T, Masaki T, et al. Inducible expression of lectin-like oxidized LDL receptor-1 in vascular endothelial cells. Circ Res. 1998;83: 322-327. doi:10.1161/01.res.83.3.322
70. Korkmaz FT, Shenoy AT, Symer EM, Baird LA, Odom CV, Arafa EI, et al. Lectin-like oxidized low-density lipoprotein receptor 1 attenuates pneumonia-induced lung injury. JCI Insight. 2022;7. doi:10.1172/jci.insight.149955
71. Ishikawa M, Ito H, Akiyoshi M, Kume N, Yoshitomi H, Mitsuoka H, et al. Lectin-like oxidized low-density lipoprotein receptor 1 signal is a potent biomarker and therapeutic target for human rheumatoid arthritis. Arthritis Rheum. 2012;64: 1024-1034. doi:10.1002/art.33452
72. Xu S, Ogura S, Chen J, Little PJ, Moss J, Liu P. LOX-1 in atherosclerosis: biological functions and pharmacological modifiers. Cell Mol Life Sci. 2013;70: 2859-2872. doi:10.1007/s00018-012-1194-z
73. Leonard J, Kepplinger D, Espina V, Gillevet P, Ke Y, Birukov KG, et al. Whole blood coagulation in an ex vivo thrombus is sufficient to induce clot neutrophils to adopt a myeloid-derived suppressor cell signature and shed soluble Lox-1. Journal of Thrombosis and Haemostasis. in revision.
74. Weidtmann A, Scheithe R, Hrboticky N, Pietsch A, Lorenz R, Siess W. Mildly Oxidized LDL Induces Platelet Aggregation Through Activation of Phospholipase A2. Arteriosclerosis, Thrombosis, and Vascular Biology. 1995;15: 1131-1138. doi:10.1161/01.ATV.15.8.1131
75. Murphy JE, Tacon D, Tedbury PR, Hadden JM, Knowling S, Sawamura T, et al. LOX-1 scavenger receptor mediates calcium-dependent recognition of phosphatidylserine and apoptotic cells. Biochem J. 2006;393: 107-115. doi:10.1042/BJ20051166
76. Ohki I, Amida H, Yamada R, Sugihara M, Ishigaki T, Tate S. Surface plasmon resonance study on functional significance of clustered organization of lectin-like oxidized LDL receptor (LOX-1). Biochimica et Biophysica Acta (BBA)-Proteins and Proteomics. 2011;1814: 345-354. doi:10.1016/j.bbapap.2010.10.006
77. Oskolkova 0, Gawlak G, Tian Y, Ke Y, Sarich N, Son S, et al. Prostaglandin E receptor-4 receptor mediates endothelial barrier-enhancing and anti-inflammatory effects of oxidized phospholipids. FASEB J. 2017;31: 4187-4202. doi:10.1096/fj.201601232RR
78. Rajabinejad M, Salari F, Gorgin Karaji A, Rezaiemanesh A. The role of myeloid-derived suppressor cells in the pathogenesis of rheumatoid arthritis; anti- or pro-inflammatory cells? Artificial Cells, Nanomedicine, and Biotechnology. 2019;47: 4149-4158. doi:10.1080/21691401.2019.1687504
79. Sacchi A, Grassi G, Notari S, Gili S, Bordoni V, Tartaglia E, et al. Expansion of Myeloid Derived Suppressor Cells Contributes to Platelet Activation by L-Arginine Deprivation during SARS-CoV-2 Infection. Cells. 2021;10: 2111. doi:10.3390/cells10082111
80. Steele NG, Carpenter ES, Kemp SB, Sirihorachai VR, The S, Delrosario L, et al. Multimodal mapping of the tumor and peripheral blood immune landscape in human pancreatic cancer. Nat Cancer. 2020;1: 1097-1112. doi:10.1038/s43018-020-00121-4
81. Bryant AJ, Fu C, Lu Y, Brantly ML, Mehrad B, Moldawer LL, et al. A checkpoint on innate myeloid cells in pulmonary arterial hypertension. Pulm Circ. 2019;9: 2045894018823528. doi:10.1177/2045894018823528
82. Glodde N, Bald T, van den Boorn-Konijnenberg D, Nakamura K, O′Donnell JS, Szczepanski S, et al. Reactive Neutrophil Responses Dependent on the Receptor Tyrosine Kinase c-MET Limit Cancer Immunotherapy. Immunity. 2017;47: 789-802.e9. doi:10.1016/j.immuni.2017.09.012
83. Chen M, Kakutani M, Naruko T, Ueda M, Narumiya S, Masaki T, et al. Activation-Dependent Surface Expression of LOX-1 in Human Platelets | Elsevier Enhanced Reader. Biochemical and Biophysical Research Communications. 2001;282: 153-158. doi:10.1006/bbrc.2001.4516
84. Shen M-Y, Chen F-Y, Hsu J-F, Fu R-H, Chang C-M, Chang C-T, et al. Plasma L5 levels are elevated in ischemic stroke patients and enhance platelet aggregation. Blood. 2016;127: 1336-1345.
85. Parnell GP, Tang BM, Nalos M, Armstrong NJ, Huang SJ, Booth DR, et al. Identifying Key Regulatory Genes in the Whole Blood of Septic Patients to Monitor Underlying Immune Dysfunctions. Shock. 2013;40: 166. doi:10.1097/SHK.0b013e31829ee604
86. Bruni F, Pasqui AL, Pastorelli M, Bova G, Cercignani M, Palazzuoli A, et al. Different Effect of Statins on Platelet Oxidized-LDLReceptor (CD36 and LOX-1) Expressionin Hypercholesterolemic Subjects. Clin Appl Thromb Hemost. 2005;11: 417-428. doi:10.1177/107602960501100408
87. Hara Y, Kusumi Y, Mitsumata M, Li X-K, Fujino M. Lysophosphatidylcholine upregulates LOX-1, chemokine receptors, and activation-related transcription factors in human T-cell line Jurkat. J Thromb Thrombolysis. 2008;26: 113-118. doi:10.1007/s11239-007-0158-x
88. Liu P, Zhu W, Chen C, Yan B, Zhu L, Chen X, et al. The mechanisms of lysophosphatidylcholine in the development of diseases. Life Sciences. 2020;247: 117443. doi:10.1016/j.lfs.2020.117443
89. Ren J, Lin J, Yu L, Yan M. Lysophosphatidylcholine: Potential Target for the Treatment of Chronic Pain. International Journal of Molecular Sciences. 2022;23: 8274. doi:10.3390/ijms23158274
90. Hinzman CP, Singh B, Bansal S, Li Y, Iliuk A, Girgis M, et al. A multi-omics approach identifies pancreatic cancer cell extracellular vesicles as mediators of the unfolded protein response in normal pancreatic epithelial cells. J Extracell Vesicles. 2022;11: e12232. doi:10.1002/jev2.12232
91. Murase T, Kume N, Kataoka H, Minami M, Sawamura T, Masaki T, et al. Identification of Soluble Forms of Lectin-Like Oxidized LDL Receptor-1. ATVB. 2000;20: 715-720. doi:10.1161/01.ATV.20.3.715
92. Biocca S, Arcangeli T, Tagliaferri E, Testa B, Vindigni G, Oteri F, et al. Simulative and experimental investigation on the cleavage site that generates the soluble human LOX-1. Arch Biochem Biophys. 2013;540: 9-18. doi:10.1016/j.abb.2013.10.001
93. Crisford H, Sapey E, Stockley RA. Proteinase 3; a potential target in chronic obstructive pulmonary disease and other chronic inflammatory diseases. Respir Res. 2018;19: 180. doi:10.1186/s12931-018-0883-z
94. Korkmaz B, Horwitz MS, Jenne DE, Gauthier F. Neutrophil Elastase, Proteinase 3, and Cathepsin G as Therapeutic Targets in Human Diseases. Pharmacol Rev. 2010;62: 726-759. doi:10.1124/pr.110.002733
95. Caescu CI, Jeschke GR, Turk BE. Active site determinants of substrate recognition by the metalloproteinases TACE and ADAM10. Biochem J. 2009;424: 79-88. doi:10.1042/BJ20090549
96. Konstantopoulos K, Neelamegham S, Burns AR, Hentzen E, Kansas GS, Snapp KR, et al. Venous levels of shear support neutrophil-platelet adhesion and neutrophil aggregation in blood via P-selectin and beta2-integrin. Circulation. 1998;98: 873-882. doi:10.1161/01.cir.98.9.873
97. Gasecka A, Nieuwland R, van der Pol E, Hajji N, Ćwiek A, Pluta K, et al. P2Y12 antagonist ticagrelor inhibits the release of procoagulant extracellular vesicles from activated platelets. Cardiol J. 2019;26: 782-789. doi:10.5603/CJ.a2018.0045
98. Schnoor B, Papa A-L. Lyophilized platelets inhibit platelet aggregation with simultaneous paradoxical promotion of platelet adhesion. Front Bioeng Biotechnol. 2022;10: 941817. doi:10.3389/fbioe.2022.941817
99. Dircks BH, Mischke R, Schuberth H-J. Platelet-neutrophil aggregate formation in blood samples from dogs with systemic inflammatory disorders. Am J Vet Res. 2012;73: 939-945. doi:10.2460/ajvr.73.7.939
100. Korkmaz B, Attucci S, Juliano MA, Kalupov T, Jourdan M-L, Juliano L, et al. Measuring elastase, proteinase 3 and cathepsin G activities at the surface of human neutrophils with fluorescence resonance energy transfer substrates. Nat Protoc. 2008;3: 991-1000. doi:10.1038/nprot.2008.63
101. Grzywa R, Lesner A, Korkmaz B, Sieńczyk M. Proteinase 3 phosphonic inhibitors. Biochimie 2019;166: 142-149. doi:10.1016/j.biochi.2019.03.005
102. Harding SA, Mallett SV, Peachey TD, Cox DJ. Use of heparinase modified thrombelastography in liver transplantation. British Journal of Anaesthesia. 1997;78: 175-179. doi:10.1093/bja/78.2.175
103. Hoemann CD, Chen G, Marchand C, Tran-Khanh N, Thibault M, Chevrier A, et al. Scaffold-Guided Subchondral Bone Repair: Implication of Neutrophils and Alternatively Activated Arginase-1+ Macrophages. Am J Sports Med. 2010;38: 1845-1856. doi:10.1177/0363546510369547
104. Hoemann CD, Marchand C, Rivard G-E, El-Gabalawy H, Poubelle PE. Effect of chitosan and coagulation factors on the wound repair phenotype of bioengineered blood clots. International Journal of Biological Macromolecules. 2017;104: 1916-1924. doi:10.1016/j.ijbiomac.2017.04.114
105. Norris PC, Libreros S, Chiang N, Serhan CN. A cluster of immunoresolvents links coagulation to innate host defense in human blood. Sci Signal. 2017;10: eaan1471. doi:10.1126/scisignal.aan1471
106. Mitsuoka H, Kume N, Hayashida K, Inui-Hayashiada A, Aramaki Y, Toyohara M, et al. Interleukin 18 stimulates release of soluble lectin-like oxidized LDL receptor-1 (sLOX-1). Atherosclerosis. 2009;202: 176-182. doi:10.1016/j.atherosclerosis.2008.04.002

What is claimed is:

1. A method of generating, ex vivo production of soluble Lox-1 (sLox-1), comprising:
introducing a sample containing blood into a device;
adding a coagulation enhancing material in the sample to form a cultured blood clot;
incubating the cultured blood clot in the device at a temperature greater than 25° C. and less than 45° C. for at least 2 hours to allow production of Lox-1 from neutrophils of blood and to shed the sLox-1 outside the cultured blood clot; and
collecting sLox-1 shedded in the device, wherein the method is configured to shed sLox-1 more than fresh blood.

2. The method of claim 1, wherein an additional enhancing material comprising a lipopolysaccharide (LPS) or phorbol myristate acetate and configured to modulate sLox-1 is added to the cultured blood clot.

3. The method of claim 1, wherein the cultured blood clot is configured to produce one or more interleukins and/or cytokines.

4. The method of claim 1, wherein addition of the coagulation enhancing material in the device spikes shedding of sLox-1 into the device by about 20% to 60% more compared to a cultured blood clot free of the coagulation enhancing material.

5. The method of claim 1, wherein the method is configured to shed 0.2 ng to 50 ng of the sLox-1 per ml of the blood sample.

6. The method of claim 1, wherein the method is configured to produce an autologous sLox-1.

7. The method of claim 1, the device is incubated for a time-period ranging from at least 2 hours to 18 hours.

8. The method of claim 1, wherein the device comprises a thrombus device.

9. The method of claim 1, wherein the neutrophils form a synapse with lymphocytes of the blood.

10. The method of claim 1, wherein the neutrophils comprise a marker comprising CD15+.

11. The method of claim 1, wherein the neutrophils of the blood are configured to undergo polymorphonuclear Myeloid-derived suppressor cells (PMN-MDSC) polarization.

12. The method of claim 1, wherein a cultured clot serum of the cultured blood clot is configured to provide an endothelial barrier-enhancing effect more than a fresh clot serum.

13. The method of claim 12, wherein the endothelial barrier-enhancing effect is about 1.5 to 15 times greater than the fresh clot serum.

14. The method of claim 1, wherein the method is configured to detect a drug response in whole blood.

15. The method of claim 1, wherein the device comprises an anionic clot-activator.

16. The method of claim 3, wherein one or more interleukins and/or cytokines comprise IL-8, IL-6, TNF or a combination thereof.

17. The method of claim 1, wherein the cultured blood clot comprises OLR1.

18. The method of claim 9, wherein the cultured blood clot comprises about 1.5 to 5 times more Lox-1+/CD15+containing neutrophils than the fresh blood clot.

19. The method of claim 1, wherein the method is configured to estimate an amount of active alpha-1 antitrypsin in a sample to detect a disease in a subject.

20. The method of claim 1, wherein the temperature is in a range of about 30° C. to about 42° C.

21. The method of claim 1, wherein addition of the coagulation enhancing material in the device comprises chitosan.

22. The method of claim 14, wherein the method is configured to monitor the sLox-1 production before and after a patient treatment or a clinical trialing with a therapy that influences neutrophil or platelet counts.
